(19) United States
(12) Patent Application Publication
(10) Pub. No.: US 2024/0083876 A1
(43) Pub. Date: Mar. 14, 2024

(54) ORGANIC ELECTROLUMINESCENT COMPOUNDS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Jae-Hoon SHIM, Gyeonggi-do (KR); Young-Mook LIM, Gyeonggi-do (KR)

(21) Appl. No.: 18/510,976

(22) Filed: Nov. 16, 2023

Related U.S. Application Data
(63) Continuation of application No. 17/532,270, filed on Nov. 22, 2021, now abandoned.
Application No. 17/532,270 is a continuation of application No. 15/768,584, filed on Apr. 16, 2018, now abandoned.

(30) Foreign Application Priority Data
Oct. 22, 2015 (KR) 10-2015-0147412
Aug. 24, 2016 (KR) 10-2016-0107527

(51) Int. Cl. C07D 401/14; H10K 50/12; H10K 85/40; H10K 85/60

(52) U.S. Cl. CPC C07D 401/14; H10K 50/12; H10K 85/40; H10K 85/615; H10K 85/626; H10K 85/654; H10K 85/6572; H10K 85/6574; H10K 85/6576; H10K 50/11

(57) ABSTRACT

The present disclosure relates to an organic electroluminescent compound and an organic electroluminescent device comprising the same. An organic electroluminescent device can have a good lifespan by using the organic electroluminescent compound of the present disclosure.

### CROSS-REFERENCE TO RELATED APPLICATIONS

This application is a continuation of U.S. patent application Ser. No. 17/532,270, filed Nov. 22, 2021, which is a continuation of U.S. patent application Ser. No. 15/768,584, filed Apr. 16, 2018, which is the national stage entry, filed under 35 U.S.C. § 371, of International Patent Application No. PCT/KR2016/011040, filed Oct. 4, 2016, each of which is incorporated by reference herein in its entirety.

### TECHNICAL FIELD

The present disclosure relates to an organic electroluminescent compound and an organic electroluminescent device comprising the same.

### BACKGROUND ART

An electroluminescent (EL) device is a self-light-emitting device which has advantages in that it provides a wider viewing angle, a greater contrast ratio, and a faster response time. An organic EL device was first developed by Eastman Kodak, by using small aromatic diamine molecules and aluminum complexes as materials to form a light-emitting layer [Appl. Phys. Lett. 51, 913, 1987].

The organic EL device (OLED) converts electric energy into light when electricity is applied to an organic light-emitting material(s). Generally, the organic EL device has a structure comprising an anode, a cathode, and an organic layer disposed between the anode and the cathode. The organic layer of the organic EL device comprises a hole injection layer, a hole transport layer, an electron blocking layer, a light-emitting layer (comprising a host material and a dopant material), an electron buffering layer, a hole blocking layer, an electron transport layer, an electron injection layer, etc. Depending on its function, materials for forming the organic layer can be classified as a hole injection material, a hole transport material, an electron blocking material, a light-emitting material, an electron buffering material, a hole blocking material, an electron transport material, an electron injection material, etc. When a voltage is applied to the organic EL device, holes and electrons are injected from an anode and a cathode, respectively, to the light-emitting layer. Excitons having high energy are formed by recombinations between the holes and the electrons. The energy puts the organic light-emitting compound in an excited state, and the decay of the excited state results in a relaxation of the energy level into a ground state, accompanied by light-emission.

The most important factor determining luminous efficiency in the organic EL device is light-emitting materials. The light-emitting material needs to have high quantum efficiency, high electron mobility, and high hole mobility. Furthermore, the light-emitting layer formed by the light-emitting material needs to be uniform and stable. Depending on the colors visualized by light-emission, the light-emitting materials can be classified as a blue-, green-, or red-emitting material, and can additionally include a yellow- or orange-emitting material. Furthermore, the light-emitting material can be classified according to its function, as a host material and a dopant material. Recently, the development of an OLED providing high efficiency and a long lifespan is urgent. In particular, considering EL requirements for a middle or large-sized OLED panel, materials showing better performance than conventional ones must be urgently developed. In order to achieve the development, a host material which plays a role as a solvent in a solid state and transfers energy, should have high purity, and an appropriate molecular weight for being deposited under a vacuum. In addition, a host material should have high glass transition temperature and high thermal decomposition temperature to ensure thermal stability; high electrochemical stability to have a long lifespan; ease of preparation for amorphous thin film; and good adhesion to materials of adjacent layers. Furthermore, a host material should not move to an adjacent layer.

The light-emitting material can be prepared by combining a host with a dopant to improve color purity, luminous efficiency, and stability. Generally, a device showing good EL performances comprises a light-emitting layer prepared by combining a host with a dopant. The host material greatly influences the efficiency and lifespan of the EL device when using a host/dopant system, and thus its selection is important.

Japanese Patent No. 5018138 and Korean Patent Application Laying-Open No. 10-2010-0108924 disclose an organic electroluminescent device using benzo[c]carbazole derivatives as a host material, Japanese Patent No. 5673362 discloses an organic electroluminescent device using benzo[c]carbazole derivatives as an electron transport material, International Publication No. WO 2010/113726 A1 discloses an organic electroluminescent device using a compound having an indolocarbazole skeleton to which a triazinyl pyridine is bonded, as a host material. Korean Patent Application Laying-Open No. 10-2013-0066554 discloses an organic electroluminescent device using aza-benzo[c]carbazole derivatives wherein pyridine is fused to carbazole, as an electron transport material. However, they do not specifically disclose an organic electroluminescent device using a compound having a benzo[c]carbazole skeleton to which a triazinyl pyridine is bonded, as a host material.

### DISCLOSURE OF THE INVENTION

### Problems to be Solved

The objective of the present disclosure is to provide an organic electroluminescent compound, which is effective in preparing an organic electroluminescent device having a remarkably improved lifespan.

### Solution to Problems

As a result of an earnest study for solving the above-described problems, the present inventors found that the above objective can be achieved by an organic electroluminescent compound represented by the following formula 1 and have come to complete the present disclosure.

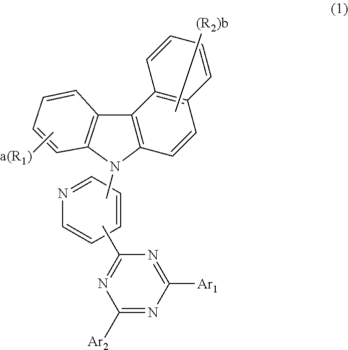

In formula 1,
Ar₁ and Ar₂, each independently, represent a substituted or unsubstituted (C6-C30)aryl, a substituted or unsubstituted (C3-C30)cycloalkyl, or a substituted or unsubstituted (3 to 30-membered)heteroaryl;
R₁ and R₂, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30) alkynyl, a substituted or unsubstituted (C3-C30)cycloalkyl, a substituted or unsubstituted (C6-C60)aryl, a substituted or unsubstituted (3 to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino;
a represents an integer of 1 to 4; b represents an integer of 1 to 6; when a or b is an integer of 2 or more, each of R₁ or each of R₂ may be the same or different; and
the heteroaryl contains at least one heteroatom selected from B, N, O, S, Si, and P.

### Effects of the Invention

An organic electroluminescent device can have a good lifespan by using the organic electroluminescent compounds of the present disclosure as a host material.

### EMBODIMENTS OF THE INVENTION

Hereinafter, the present disclosure will be described in detail. However, the following description is intended to explain the disclosure, and is not meant in any way to restrict the scope of the disclosure.

Hereinafter, the organic electroluminescent compound of formula 1 of the present disclosure will be described in detail.

The compound of formula 1 of the present disclosure may be represented by any one of the following formulae 2 to 6:

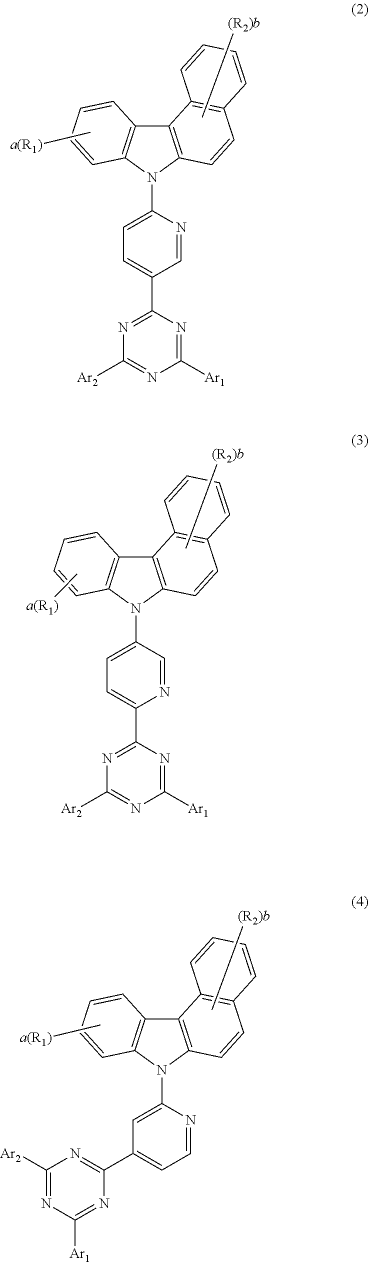

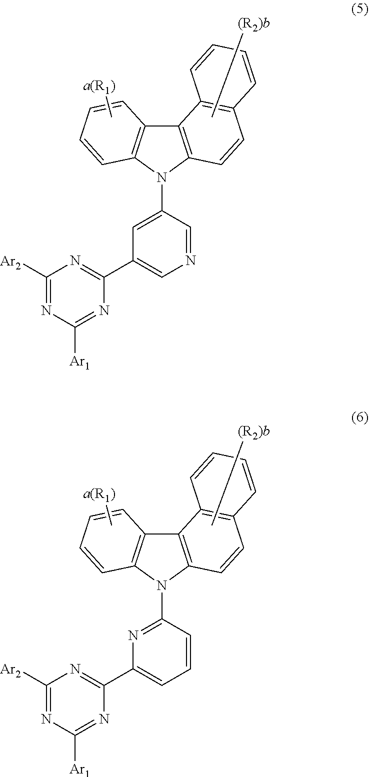

In formulae 2 to 6,
Ar₁, Ar₂, R₁, R₂, a and b are as defined in formula 1 above.

In formula 1, Ar₁ and Ar₂, each independently, may represent a substituted or unsubstituted (C6-C30)aryl, a substituted or unsubstituted (C3-C30)cycloalkyl, or a substituted or unsubstituted (3 to 30-membered)heteroary; preferably, each independently, may represent a substituted or unsubstituted (C6-C18)aryl; more preferably, each independently, may represent an unsubstituted (C6-C18)aryl. Specifically, Ar₁ and Ar₂ each independently, may represent a substituted or unsubstituted phenyl, a substituted or unsubstituted naphthyl, a substituted or unsubstituted biphenyl, a substituted or unsubstituted terphenyl, a substituted or unsubstituted phenylnaphthyl, or a substituted or unsubstituted naphthylphenyl.

In formula 1, R₁ and R₂, each independently, may represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3 to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino; preferably, each independently, may represent hydrogen, or a substituted or unsubstituted (C6-C18)aryl; more preferably, each independently, may represent hydrogen, or an unsubstituted (C6-C18)aryl. Specifically, R₁ and R₂, each independently, may represent hydrogen, a substituted or unsubstituted phenyl, a substituted or unsubstituted biphenyl, a substituted or unsubstituted naphthyl, a substituted or unsubstituted terphenyl, a substituted or unsubstituted phenylnaphthyl, or a substituted or unsubstituted naphthylphenyl.

In formula 1, a represents an integer of 1 to 4, b represents an integer of 1 to 6; preferably, a and b, each independently, may represent 1.

Furthermore, in formula 1, the heteroaryl contains at least one heteroatom selected from B, N, O, S, Si and P; preferably, the heteroaryl may contain at least one heteroatom selected from N, O and S.

Herein, “(C1-C30)alkyl” indicates a linear or branched alkyl having 1 to 30, preferably 1 to 20, and more preferably 1 to 10 carbon atoms, and includes methyl, ethyl, n-propyl, isopropyl, n-butyl, isobutyl, tert-butyl, etc. Herein, “(C3-C30)cycloalkyl” indicates a mono- or polycyclic hydrocarbon having 3 to 30, preferabley 3 to 20, and more preferably 3 to 7 ring backbone carbon atoms. The cycloalkyl includes cyclopropyl, cyclobutyl, cyclopentyl, cyclohexyl, etc. Herein, “(3 to 7-membered)heterocycloalkyl” indicates a cycloalkyl having 3 to 7, preferably 5 to 7 ring backbone atoms including at least one heteroatom selected from B, N, O, S, Si, and P, preferably O, S, and N, and includes tetrahydrofuran, pyrrolidine, thiolan, tetrahydropyran, etc. Herein, “(C6-C30)aryl(ene)” indicates a monocyclic ring-type or fused ring-type radical derived from aromatic hydrocarbon having 6 to 30, preferabley 6 to 20, and more preferably 6 to 15 ring backbone carbon atoms. The aryl may have a spiro structure. The aryl includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. Herein, “(3 to 30-membered)heteroaryl(ene)” indicates an aryl group having 3 to 30 ring backbone atoms including at least one, preferably 1 to 4, heteroatom selected from the group consisting of B, N, O, S, Si, and P; may be a monocyclic ring, or a fused ring condensed with at least one benzene ring; may be partially saturated; may be one formed by linking at least one heteroaryl or aryl group to a heteroaryl group via a single bond(s); may have a spiro structure; and includes a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenothiazinyl, phenanthridinyl, benzodioxolyl, dihydroacridinyl, etc. Further, “halogen” includes F, Cl, Br, and I.

Furthermore, herein, “substituted” in the expression, “substituted or unsubstituted,” means that a hydrogen atom in a certain functional group is replaced with another atom or group, i.e. a substituent. The substituent of the substituted (C6-C30)aryl, the substituted (C3-C30)cycloalkyl and the substituted (3 to 30-membered)heteroaryl in Ar₁ and Ar₂, and the substituent of the substituted (C1-C30)alkyl, the substituted (C2-C30)alkenyl, the substituted (C2-C30)alkynyl, the substituted (C3-C30)cycloalkyl, the substituted (C6-C60)aryl, the substituted (3 to 30-membered)heteroaryl, the substituted tri(C1-C30)alkylsilyl, the substituted tri(C6-C30)arylsilyl, the substituted di(C1-C30)alkyl(C6-C30)arylsilyl, the substituted (C1-C30)alkyldi(C6-C30)arylsilyl, the substituted mono- or di-(C1-C30)alkylamino, and the substituted mono- or di-(06-C30)arylamino in R₁ and R₂, each independently, is at least one selected from the group consisting of deuterium, a halogen, a cyano, a carboxy, a nitro, a hydroxy, a (C1-C30)alkyl, a halo(C1-C30)alkyl, a (C2-C30)alkenyl, a (C2-C30) alkynyl, a (C1-C30)alkoxy, a (C1-C30)alkylthio, a (C3-C30)cycloalkyl, a (C3-C30)cycloalkenyl, a (3 to 7-membered)heterocycloalkyl, a (C6-C30)aryloxy, a (C6-C30)arylthio, a (5 to 30-membered)heteroaryl substituted or unsubstituted with a (C6-C30)aryl, a (C6-C30)aryl substituted or unsubstituted with a (5 to 30-membered)heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl; and preferably, each independently, may be at least one selected from the group consisting of a (C1-C6)alkyl, a (C6-C18)aryl, a (5 to 20-membered)heteroaryl and a tri(C6-C12)arylsilyl.

The compound of formula 1 of the present disclosure includes the following, but is not limited thereto:

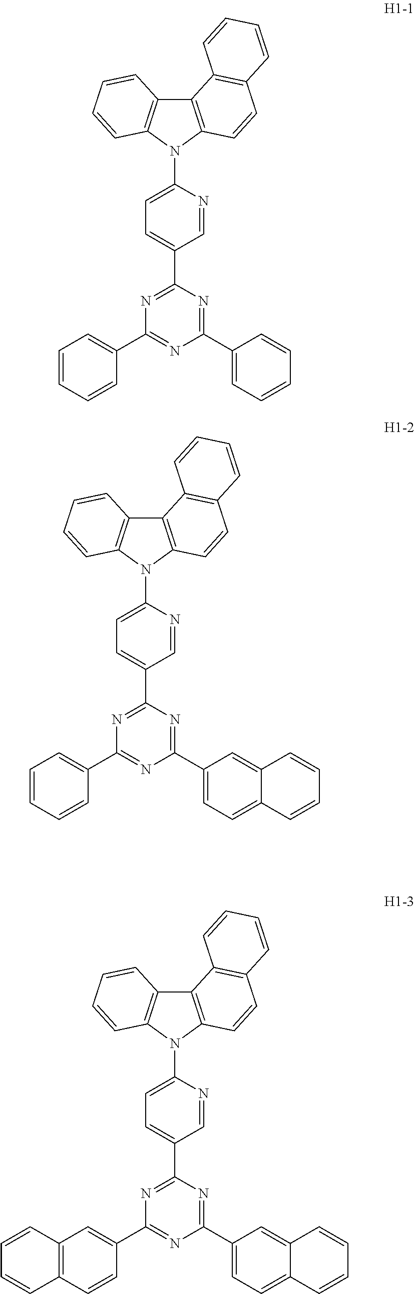 [SMILES: c1ccc(-c2nc(-c3ccccc3)nc(-c3ccc(-n4c5ccccc5c5c6ccccc6ccc54)nc3)n2)cc1 ; c1ccc(-c2nc(-c3ccc(-n4c5ccccc5c5c6ccccc6ccc54)nc3)nc(-c3ccc4ccccc4c3)n2)cc1 ; c1ccc2cc(-c3nc(-c4ccc(-n5c6ccccc6c6c7ccccc7ccc65)nc4)nc(-c4ccc5ccccc5c4)n3)ccc2c1]

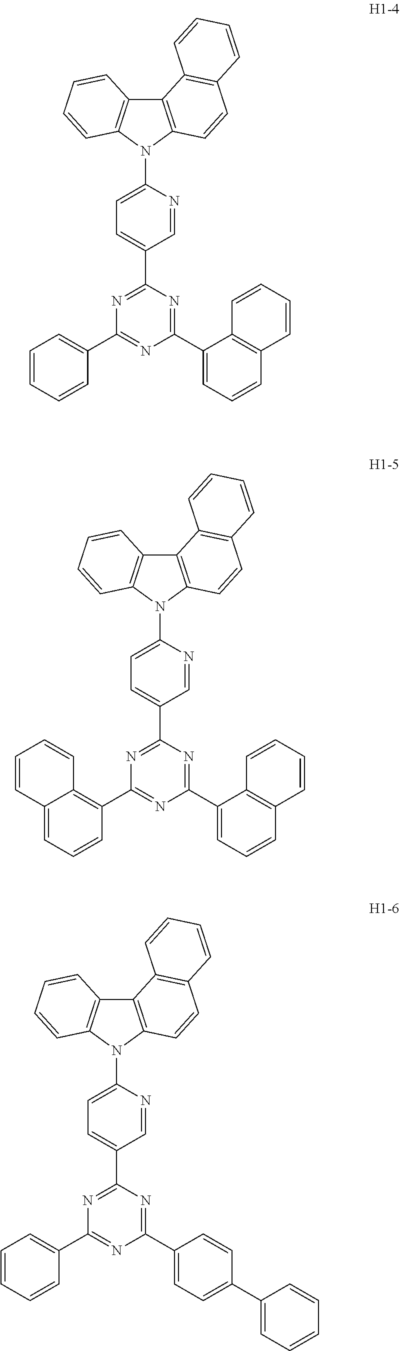 [SMILES: c1ccc(-c2nc(-c3ccc(-n4c5ccccc5c5c6ccccc6ccc54)nc3)nc(-c3cccc4ccccc34)n2)cc1 ; c1ccc2c(-c3nc(-c4ccc(-n5c6ccccc6c6c7ccccc7ccc65)nc4)nc(-c4cccc5ccccc45)n3)cccc2c1 ; c1ccc(-c2ccc(-c3nc(-c4ccccc4)nc(-c4ccc(-n5c6ccccc6c6c7ccccc7ccc65)nc4)n3)cc2)cc1]

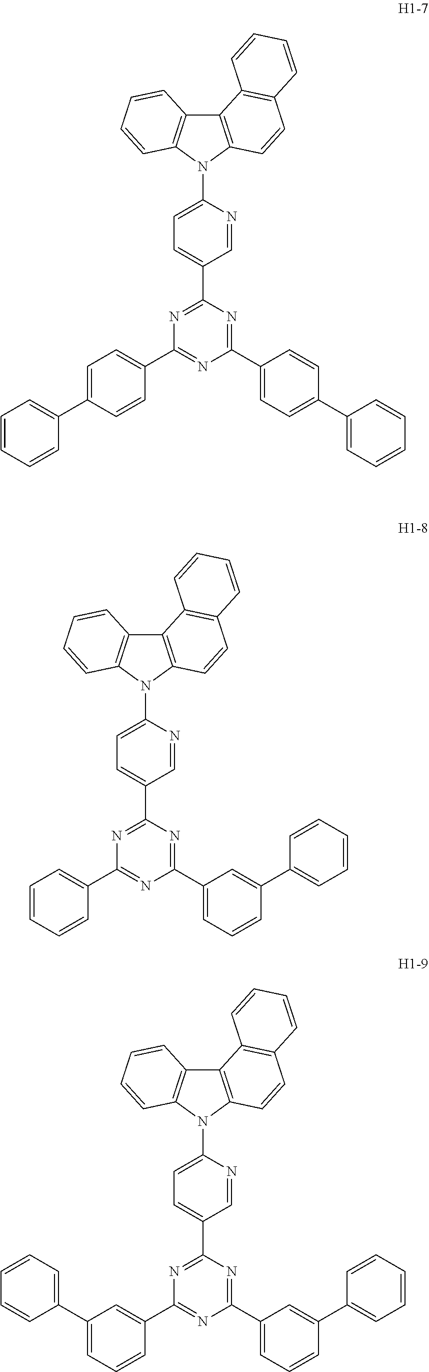 [SMILES: c1ccc(-c2ccc(-c3nc(-c4ccc(-c5ccccc5)cc4)nc(-c4ccc(-n5c6ccccc6c6c7ccccc7ccc65)nc4)n3)cc2)cc1 ; c1ccc(-c2cccc(-c3nc(-c4ccccc4)nc(-c4ccc(-n5c6ccccc6c6c7ccccc7ccc65)nc4)n3)c2)cc1 ; c1ccc(-c2cccc(-c3nc(-c4ccc(-n5c6ccccc6c6c7ccccc7ccc65)nc4)nc(-c4cccc(-c5ccccc5)c4)n3)c2)cc1]

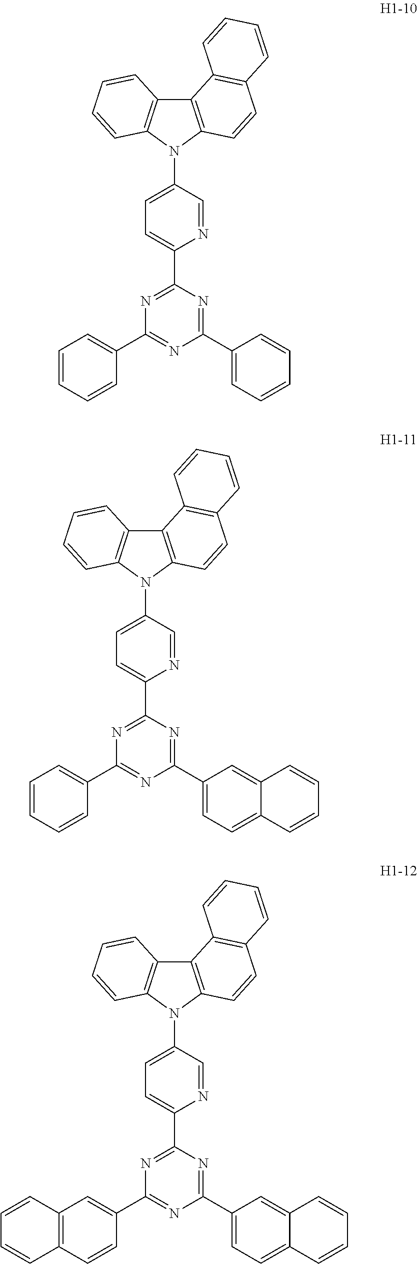 [SMILES: c1ccc(-c2nc(-c3ccccc3)nc(-c3ccc(-n4c5ccccc5c5c6ccccc6ccc54)cn3)n2)cc1 ; c1ccc(-c2nc(-c3ccc4ccccc4c3)nc(-c3ccc(-n4c5ccccc5c5c6ccccc6ccc54)cn3)n2)cc1 ; c1ccc2cc(-c3nc(-c4ccc5ccccc5c4)nc(-c4ccc(-n5c6ccccc6c6c7ccccc7ccc65)cn4)n3)ccc2c1]

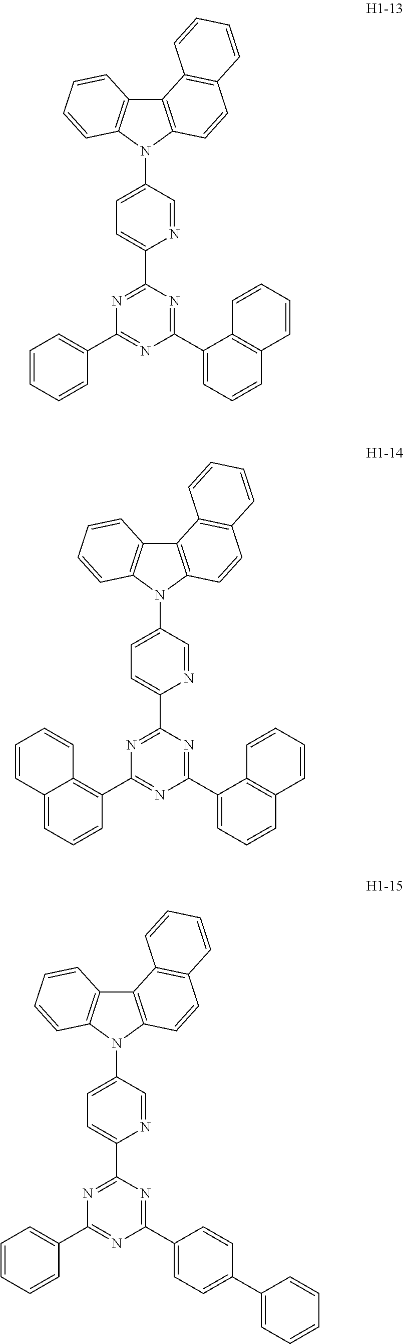 [SMILES: c1ccc(-c2nc(-c3ccc(-n4c5ccccc5c5c6ccccc6ccc54)cn3)nc(-c3cccc4ccccc34)n2)cc1 ; c1ccc2c(-c3nc(-c4ccc(-n5c6ccccc6c6c7ccccc7ccc65)cn4)nc(-c4cccc5ccccc45)n3)cccc2c1 ; c1ccc(-c2ccc(-c3nc(-c4ccccc4)nc(-c4ccc(-n5c6ccccc6c6c7ccccc7ccc65)cn4)n3)cc2)cc1]

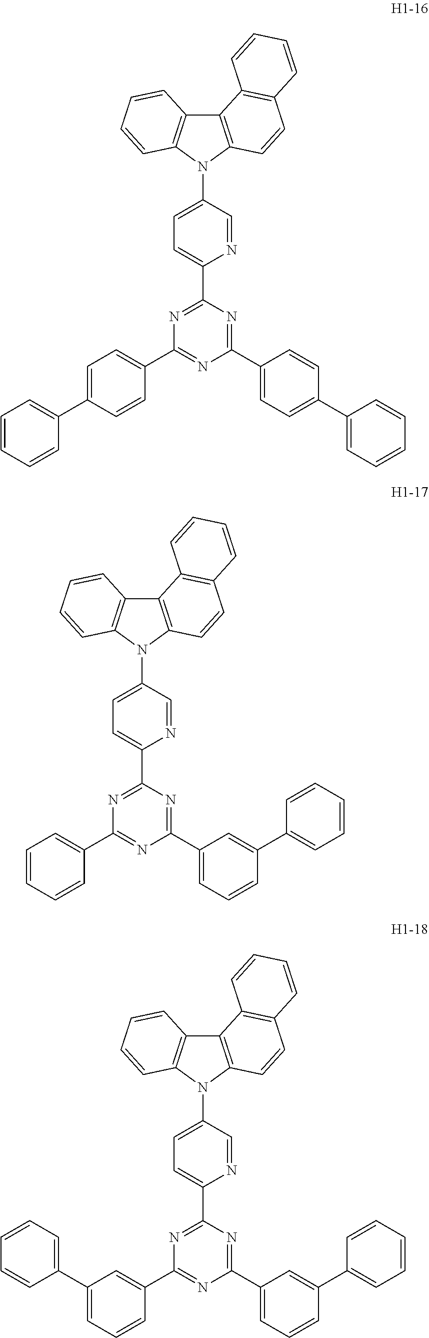 [SMILES: c1ccc(-c2ccc(-c3nc(-c4ccc(-c5ccccc5)cc4)nc(-c4ccc(-n5c6ccccc6c6c7ccccc7ccc65)cn4)n3)cc2)cc1 ; c1ccc(-c2cccc(-c3nc(-c4ccccc4)nc(-c4ccc(-n5c6ccccc6c6c7ccccc7ccc65)cn4)n3)c2)cc1 ; c1ccc(-c2cccc(-c3nc(-c4cccc(-c5ccccc5)c4)nc(-c4ccc(-n5c6ccccc6c6c7ccccc7ccc65)cn4)n3)c2)cc1]

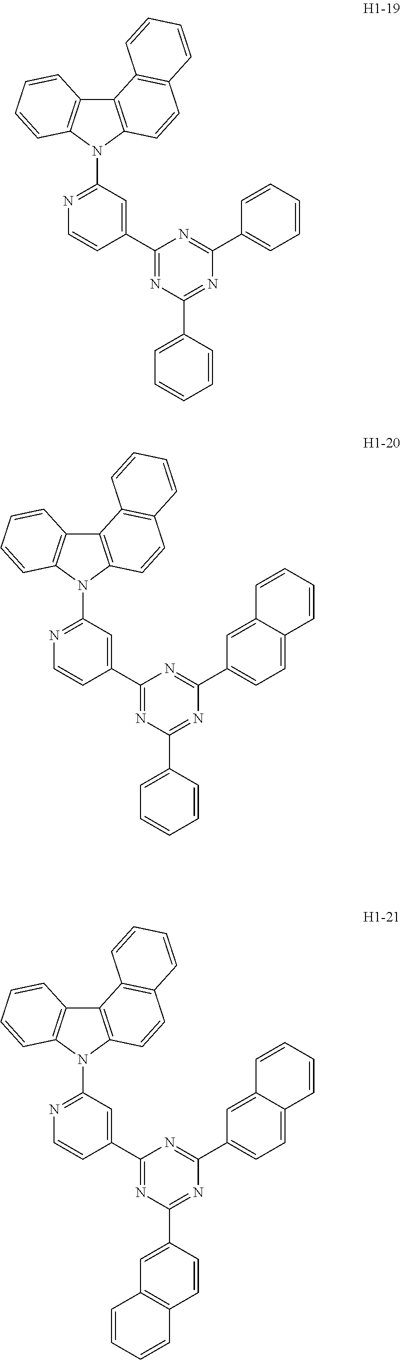 [SMILES: c1ccc(-c2nc(-c3ccccc3)nc(-c3ccnc(-n4c5ccccc5c5c6ccccc6ccc54)c3)n2)cc1 ; c1ccc(-c2nc(-c3ccnc(-n4c5ccccc5c5c6ccccc6ccc54)c3)nc(-c3ccc4ccccc4c3)n2)cc1 ; c1ccc2cc(-c3nc(-c4ccnc(-n5c6ccccc6c6c7ccccc7ccc65)c4)nc(-c4ccc5ccccc5c4)n3)ccc2c1]

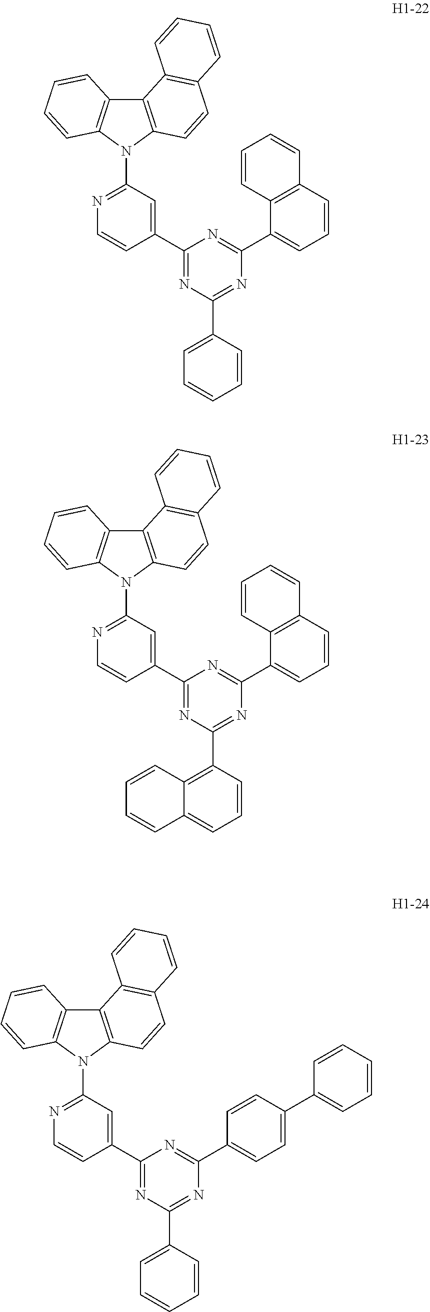 [SMILES: c1ccc(-c2nc(-c3ccnc(-n4c5ccccc5c5c6ccccc6ccc54)c3)nc(-c3cccc4ccccc34)n2)cc1 ; c1ccc2c(-c3nc(-c4ccnc(-n5c6ccccc6c6c7ccccc7ccc65)c4)nc(-c4cccc5ccccc45)n3)cccc2c1 ; c1ccc(-c2ccc(-c3nc(-c4ccccc4)nc(-c4ccnc(-n5c6ccccc6c6c7ccccc7ccc65)c4)n3)cc2)cc1]

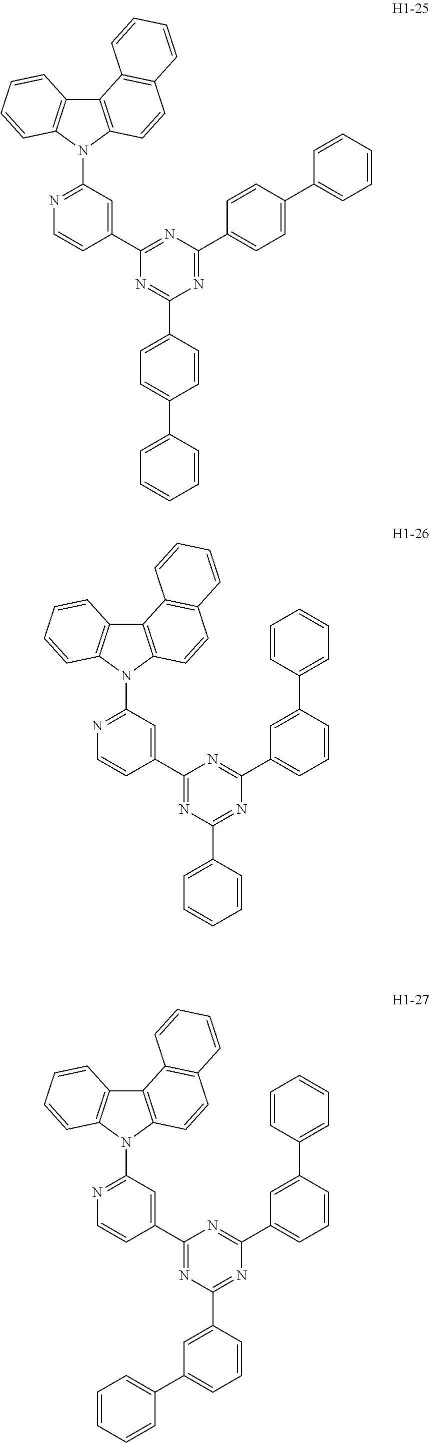

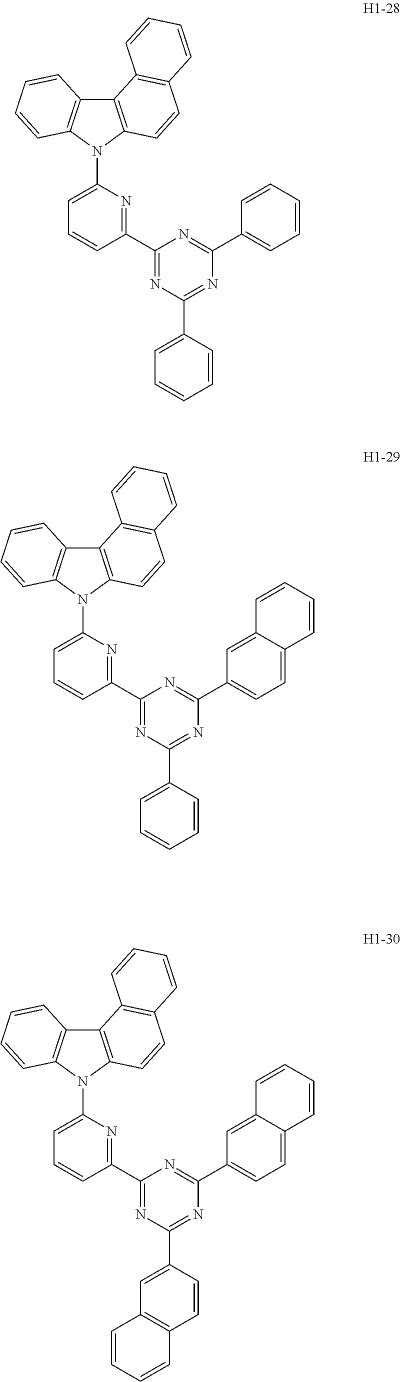 [SMILES: c1ccc(-c2nc(-c3ccccc3)nc(-c3cccc(-n4c5ccccc5c5c6ccccc6ccc54)n3)n2)cc1 ; c1ccc(-c2nc(-c3ccc4ccccc4c3)nc(-c3cccc(-n4c5ccccc5c5c6ccccc6ccc54)n3)n2)cc1 ; c1cc(-c2nc(-c3ccc4ccccc4c3)nc(-c3ccc4ccccc4c3)n2)nc(-n2c3ccccc3c3c4ccccc4ccc32)c1]

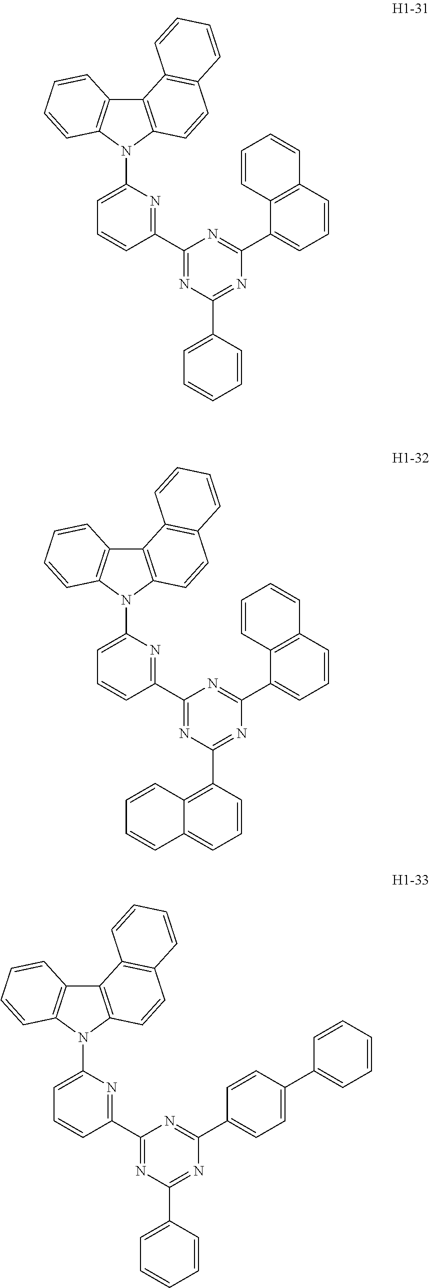 [SMILES: c1ccc(-c2nc(-c3cccc(-n4c5ccccc5c5c6ccccc6ccc54)n3)nc(-c3cccc4ccccc34)n2)cc1 ; c1cc(-c2nc(-c3cccc4ccccc34)nc(-c3cccc4ccccc34)n2)nc(-n2c3ccccc3c3c4ccccc4ccc32)c1 ; c1ccc(-c2ccc(-c3nc(-c4ccccc4)nc(-c4cccc(-n5c6ccccc6c6c7ccccc7ccc65)n4)n3)cc2)cc1]

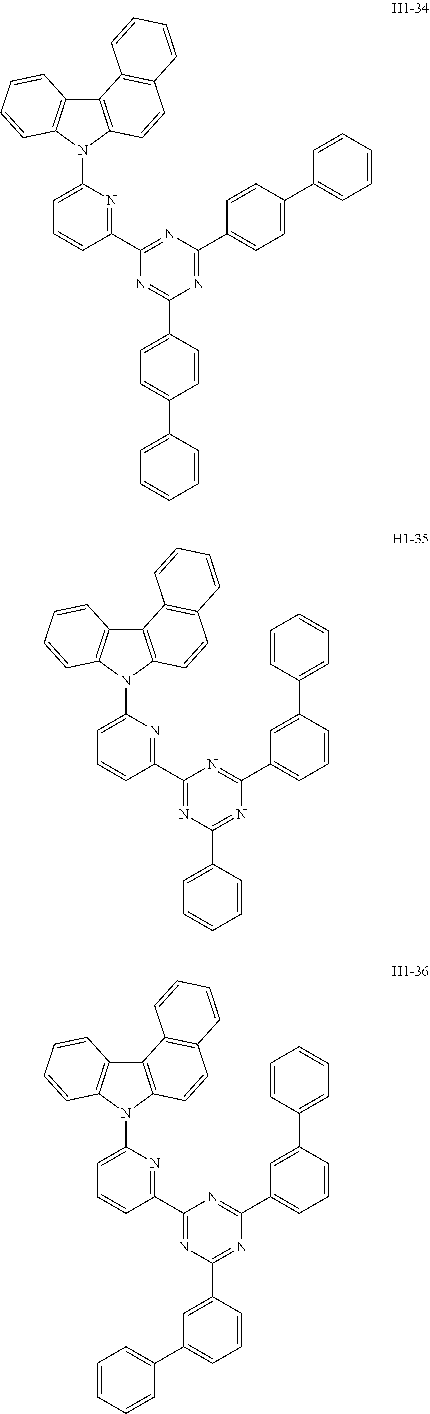 [SMILES: c1ccc(-c2ccc(-c3nc(-c4ccc(-c5ccccc5)cc4)nc(-c4cccc(-n5c6ccccc6c6c7ccccc7ccc65)n4)n3)cc2)cc1 ; c1ccc(-c2cccc(-c3nc(-c4ccccc4)nc(-c4cccc(-n5c6ccccc6c6c7ccccc7ccc65)n4)n3)c2)cc1 ; c1ccc(-c2cccc(-c3nc(-c4cccc(-c5ccccc5)c4)nc(-c4cccc(-n5c6ccccc6c6c7ccccc7ccc65)n4)n3)c2)cc1]

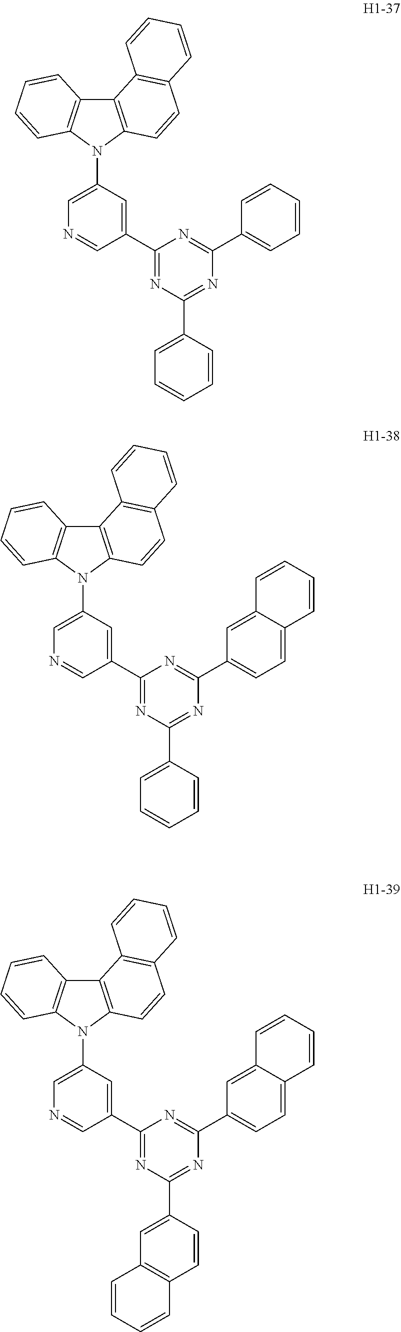 [SMILES: c1ccc(-c2nc(-c3ccccc3)nc(-c3cncc(-n4c5ccccc5c5c6ccccc6ccc54)c3)n2)cc1 ; c1ccc(-c2nc(-c3cncc(-n4c5ccccc5c5c6ccccc6ccc54)c3)nc(-c3ccc4ccccc4c3)n2)cc1 ; c1ccc2cc(-c3nc(-c4cncc(-n5c6ccccc6c6c7ccccc7ccc65)c4)nc(-c4ccc5ccccc5c4)n3)ccc2c1]

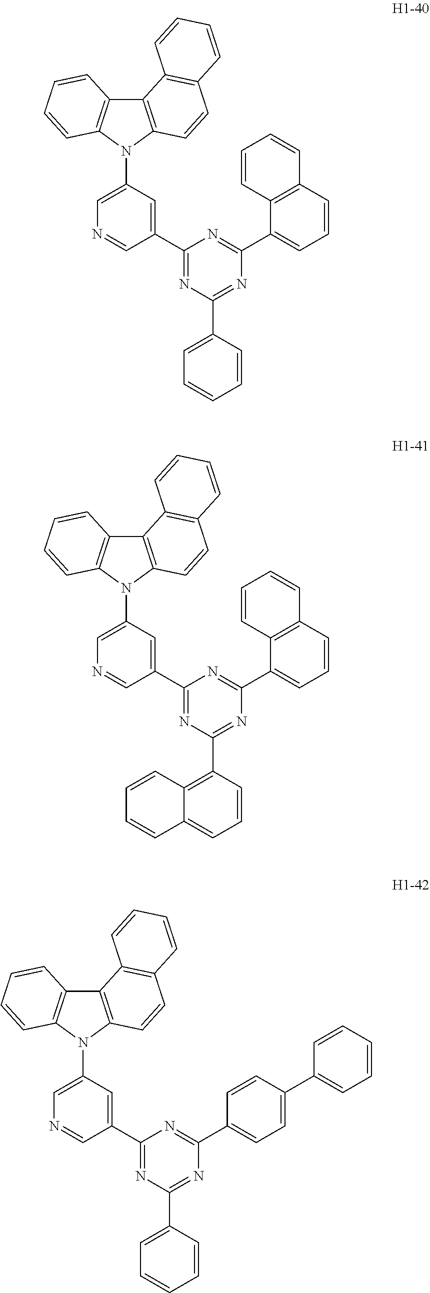 [SMILES: c1ccc(-c2nc(-c3cncc(-n4c5ccccc5c5c6ccccc6ccc54)c3)nc(-c3cccc4ccccc34)n2)cc1 ; c1ccc2c(-c3nc(-c4cncc(-n5c6ccccc6c6c7ccccc7ccc65)c4)nc(-c4cccc5ccccc45)n3)cccc2c1 ; c1ccc(-c2ccc(-c3nc(-c4ccccc4)nc(-c4cncc(-n5c6ccccc6c6c7ccccc7ccc65)c4)n3)cc2)cc1]

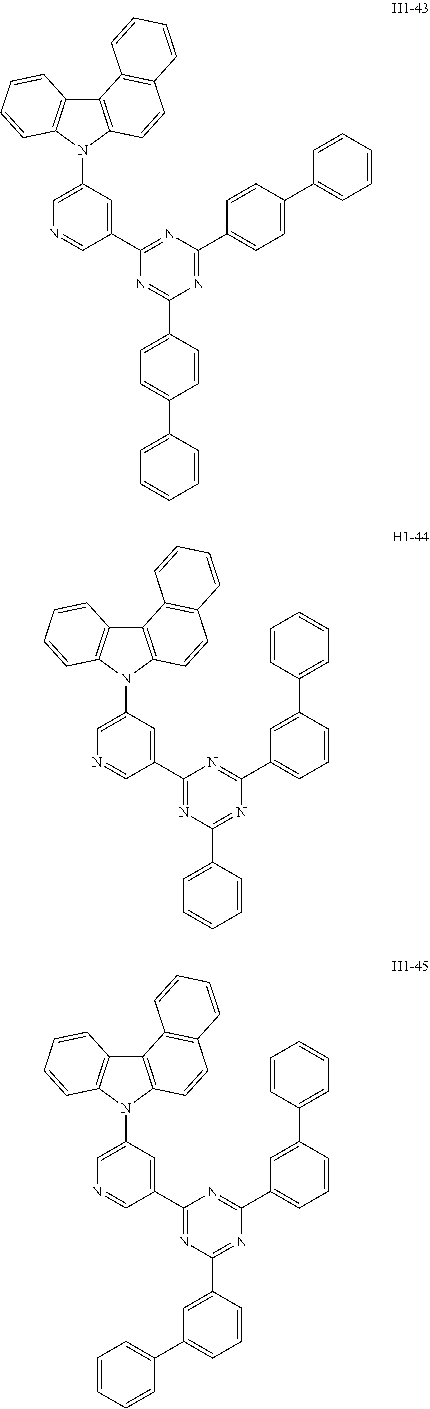

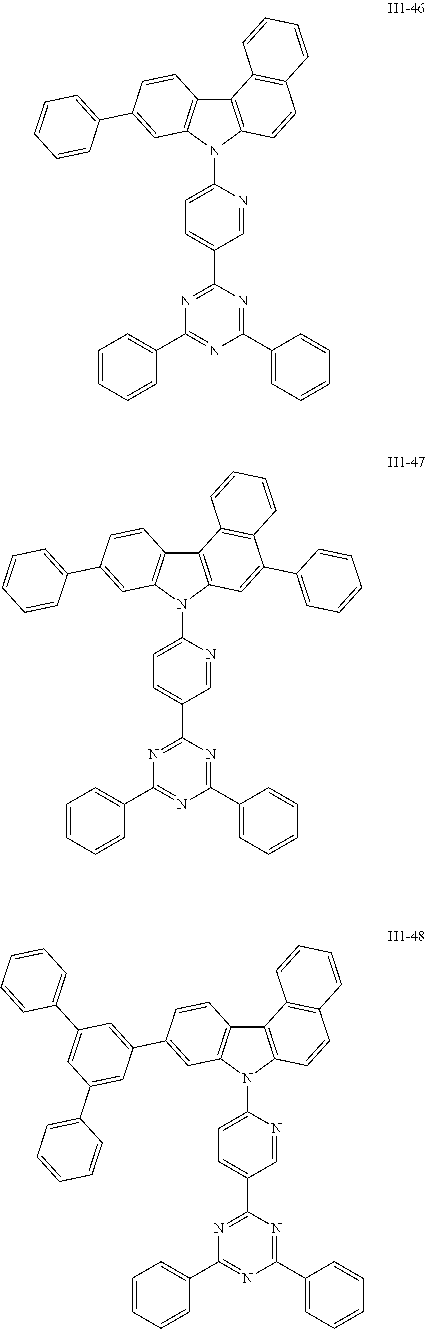 [SMILES: c1ccc(-c2ccc3c4c5ccccc5ccc4n(-c4ccc(-c5nc(-c6ccccc6)nc(-c6ccccc6)n5)cn4)c3c2)cc1 ; c1ccc(-c2ccc3c4c5ccccc5c(-c5ccccc5)cc4n(-c4ccc(-c5nc(-c6ccccc6)nc(-c6ccccc6)n5)cn4)c3c2)cc1 ; c1ccc(-c2cc(-c3ccccc3)cc(-c3ccc4c5c6ccccc6ccc5n(-c5ccc(-c6nc(-c7ccccc7)nc(-c7ccccc7)n6)cn5)c4c3)c2)cc1]

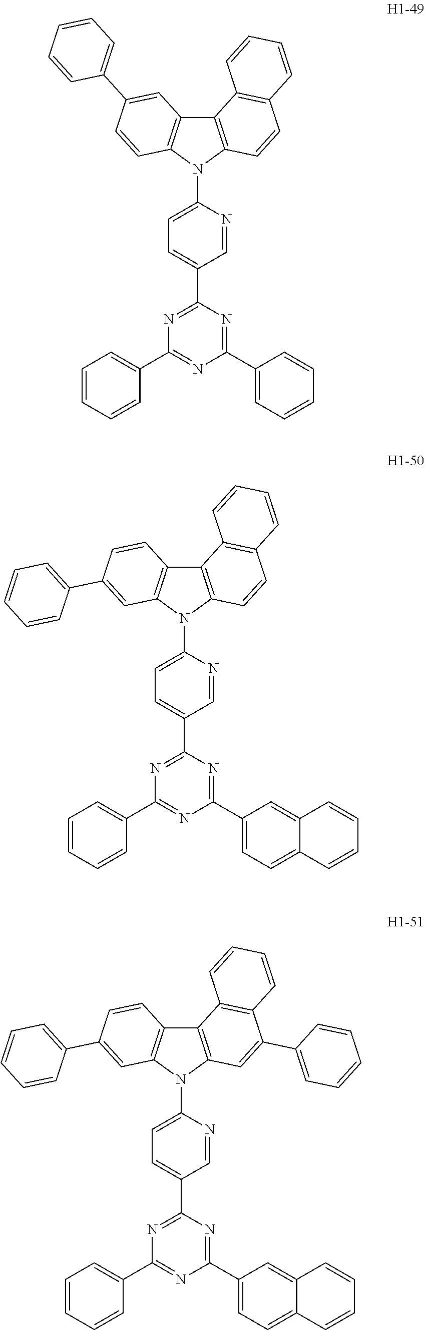 [SMILES: c1ccc(-c2ccc3c(c2)c2c4ccccc4ccc2n3-c2ccc(-c3nc(-c4ccccc4)nc(-c4ccccc4)n3)cn2)cc1 ; c1ccc(-c2ccc3c4c5ccccc5ccc4n(-c4ccc(-c5nc(-c6ccccc6)nc(-c6ccc7ccccc7c6)n5)cn4)c3c2)cc1 ; c1ccc(-c2ccc3c4c5ccccc5c(-c5ccccc5)cc4n(-c4ccc(-c5nc(-c6ccccc6)nc(-c6ccc7ccccc7c6)n5)cn4)c3c2)cc1]

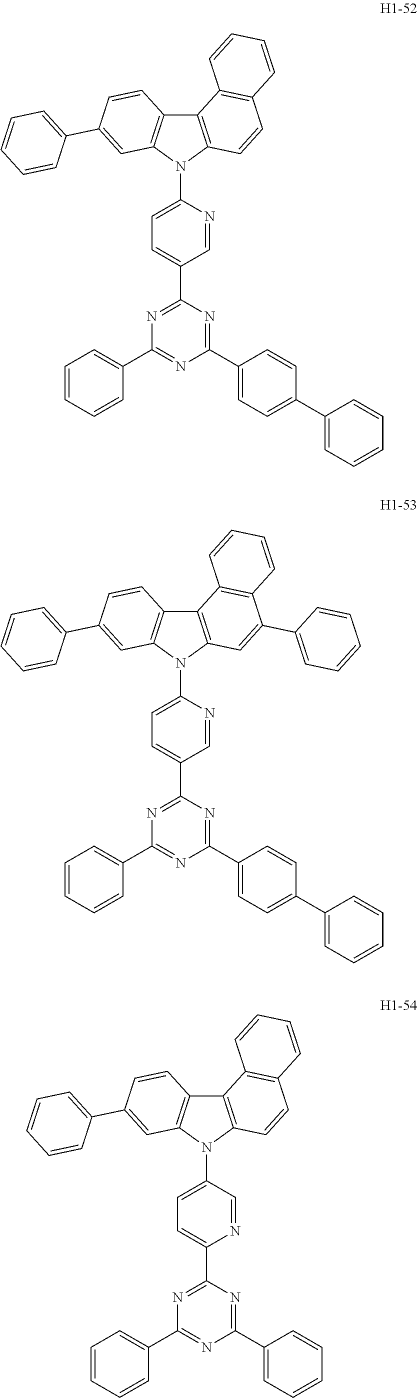 [SMILES: c1ccc(-c2ccc(-c3nc(-c4ccccc4)nc(-c4ccc(-n5c6cc(-c7ccccc7)ccc6c6c7ccccc7ccc65)nc4)n3)cc2)cc1 ; c1ccc(-c2ccc(-c3nc(-c4ccccc4)nc(-c4ccc(-n5c6cc(-c7ccccc7)ccc6c6c7ccccc7c(-c7ccccc7)cc65)nc4)n3)cc2)cc1 ; c1ccc(-c2ccc3c4c5ccccc5ccc4n(-c4ccc(-c5nc(-c6ccccc6)nc(-c6ccccc6)n5)nc4)c3c2)cc1]

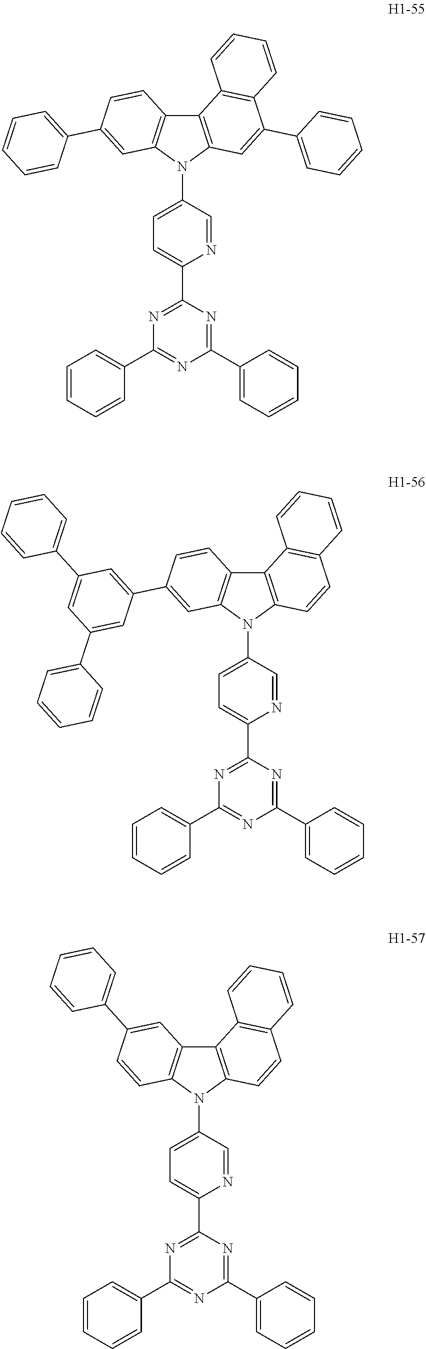 [SMILES: c1ccc(-c2ccc3c4c5ccccc5c(-c5ccccc5)cc4n(-c4ccc(-c5nc(-c6ccccc6)nc(-c6ccccc6)n5)nc4)c3c2)cc1 ; c1ccc(-c2cc(-c3ccccc3)cc(-c3ccc4c5c6ccccc6ccc5n(-c5ccc(-c6nc(-c7ccccc7)nc(-c7ccccc7)n6)nc5)c4c3)c2)cc1 ; c1ccc(-c2ccc3c(c2)c2c4ccccc4ccc2n3-c2ccc(-c3nc(-c4ccccc4)nc(-c4ccccc4)n3)nc2)cc1]

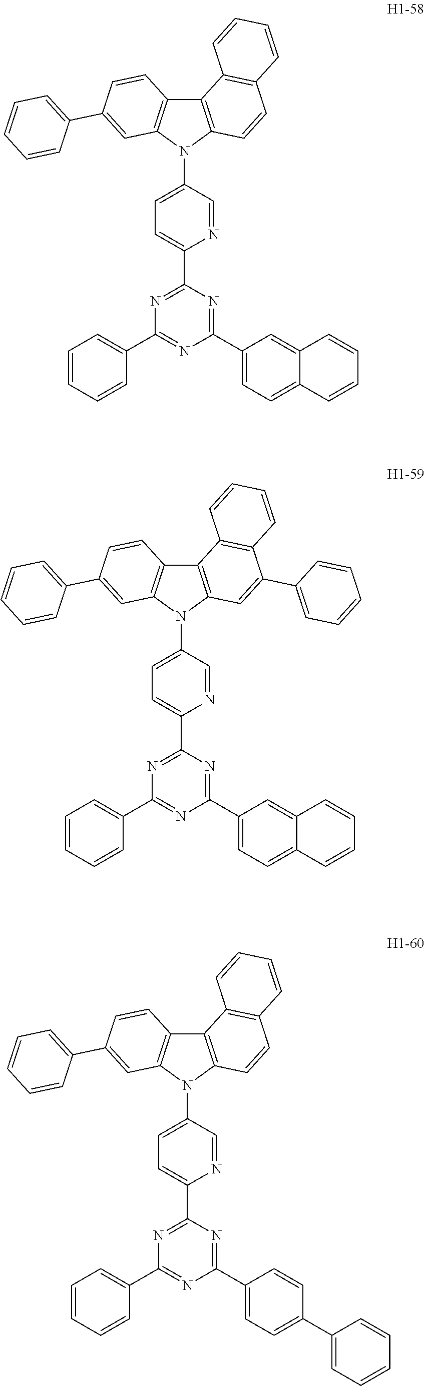 [SMILES: c1ccc(-c2ccc3c4c5ccccc5ccc4n(-c4ccc(-c5nc(-c6ccccc6)nc(-c6ccc7ccccc7c6)n5)nc4)c3c2)cc1 ; c1ccc(-c2ccc3c4c5ccccc5c(-c5ccccc5)cc4n(-c4ccc(-c5nc(-c6ccccc6)nc(-c6ccc7ccccc7c6)n5)nc4)c3c2)cc1 ; c1ccc(-c2ccc(-c3nc(-c4ccccc4)nc(-c4ccc(-n5c6cc(-c7ccccc7)ccc6c6c7ccccc7ccc65)cn4)n3)cc2)cc1]

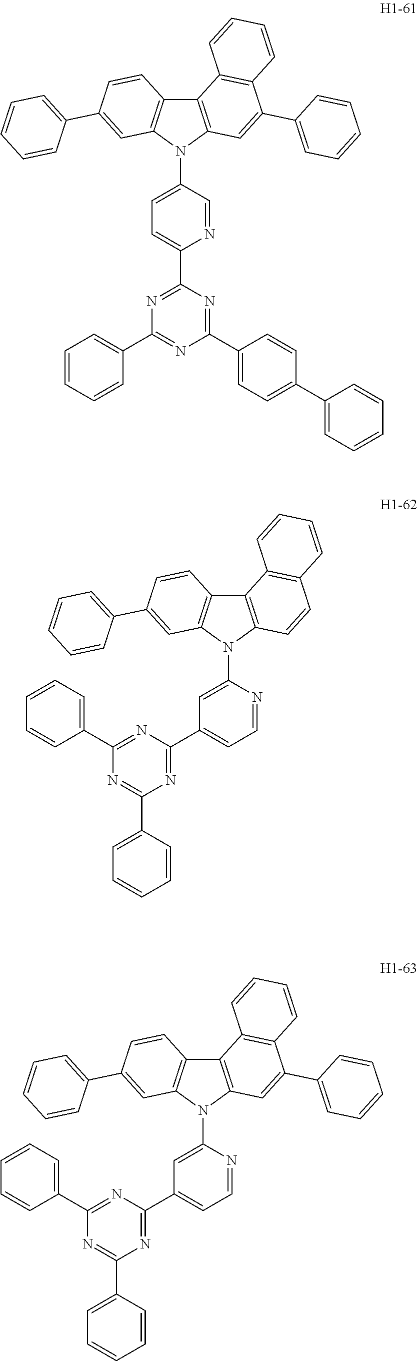 [SMILES: c1ccc(-c2ccc(-c3nc(-c4ccccc4)nc(-c4ccc(-n5c6cc(-c7ccccc7)ccc6c6c7ccccc7c(-c7ccccc7)cc65)cn4)n3)cc2)cc1 ; c1ccc(-c2ccc3c4c5ccccc5ccc4n(-c4cc(-c5nc(-c6ccccc6)nc(-c6ccccc6)n5)ccn4)c3c2)cc1 ; c1ccc(-c2ccc3c4c5ccccc5c(-c5ccccc5)cc4n(-c4cc(-c5nc(-c6ccccc6)nc(-c6ccccc6)n5)ccn4)c3c2)cc1]

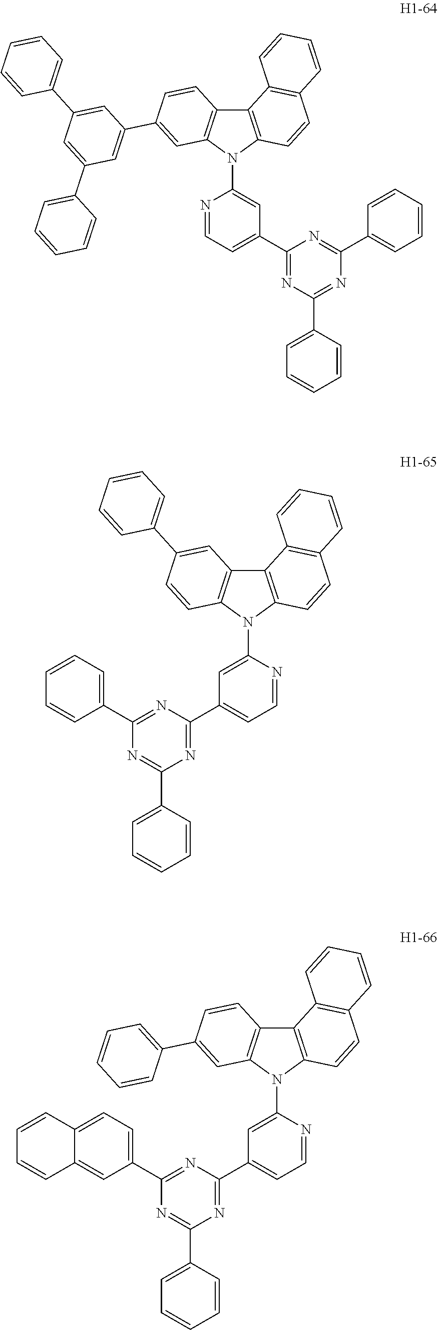 [SMILES: c1ccc(-c2cc(-c3ccccc3)cc(-c3ccc4c5c6ccccc6ccc5n(-c5cc(-c6nc(-c7ccccc7)nc(-c7ccccc7)n6)ccn5)c4c3)c2)cc1 ; c1ccc(-c2ccc3c(c2)c2c4ccccc4ccc2n3-c2cc(-c3nc(-c4ccccc4)nc(-c4ccccc4)n3)ccn2)cc1 ; c1ccc(-c2ccc3c4c5ccccc5ccc4n(-c4cc(-c5nc(-c6ccccc6)nc(-c6ccc7ccccc7c6)n5)ccn4)c3c2)cc1]

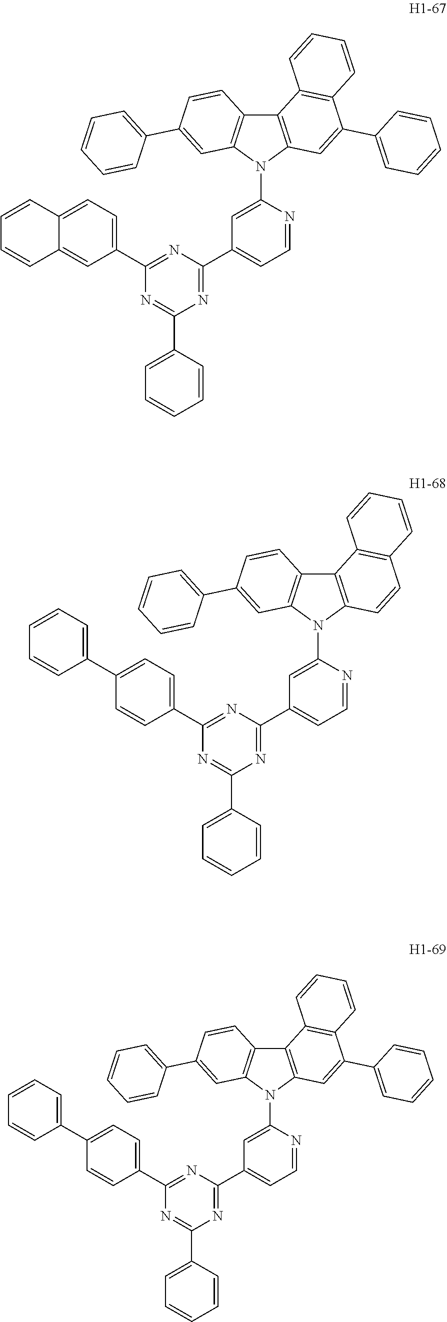 [SMILES: c1ccc(-c2ccc3c4c5ccccc5c(-c5ccccc5)cc4n(-c4cc(-c5nc(-c6ccccc6)nc(-c6ccc7ccccc7c6)n5)ccn4)c3c2)cc1 ; c1ccc(-c2ccc(-c3nc(-c4ccccc4)nc(-c4ccnc(-n5c6cc(-c7ccccc7)ccc6c6c7ccccc7ccc65)c4)n3)cc2)cc1 ; c1ccc(-c2ccc(-c3nc(-c4ccccc4)nc(-c4ccnc(-n5c6cc(-c7ccccc7)ccc6c6c7ccccc7c(-c7ccccc7)cc65)c4)n3)cc2)cc1]

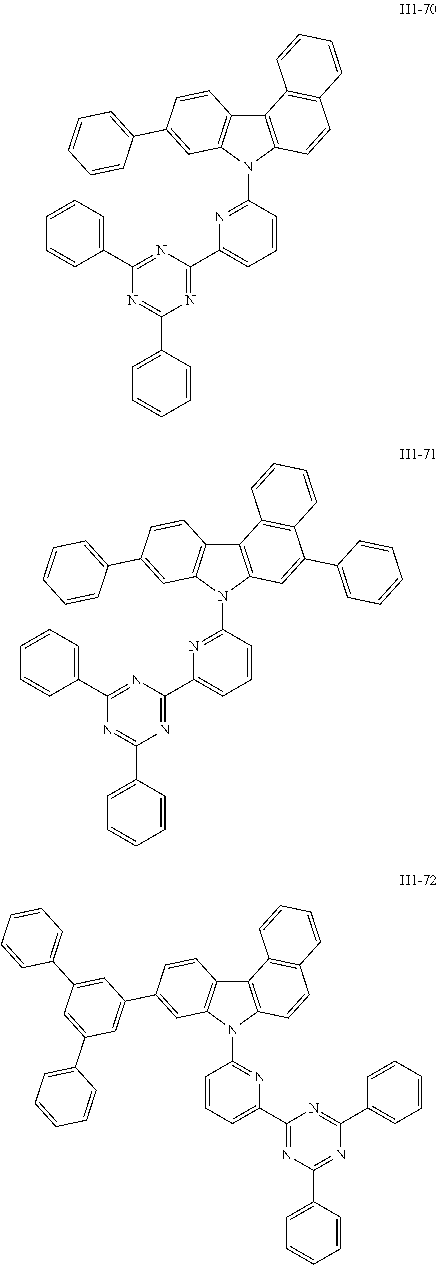 [SMILES: c1ccc(-c2ccc3c4c5ccccc5ccc4n(-c4cccc(-c5nc(-c6ccccc6)nc(-c6ccccc6)n5)n4)c3c2)cc1 ; c1ccc(-c2ccc3c4c5ccccc5c(-c5ccccc5)cc4n(-c4cccc(-c5nc(-c6ccccc6)nc(-c6ccccc6)n5)n4)c3c2)cc1 ; c1ccc(-c2cc(-c3ccccc3)cc(-c3ccc4c5c6ccccc6ccc5n(-c5cccc(-c6nc(-c7ccccc7)nc(-c7ccccc7)n6)n5)c4c3)c2)cc1]

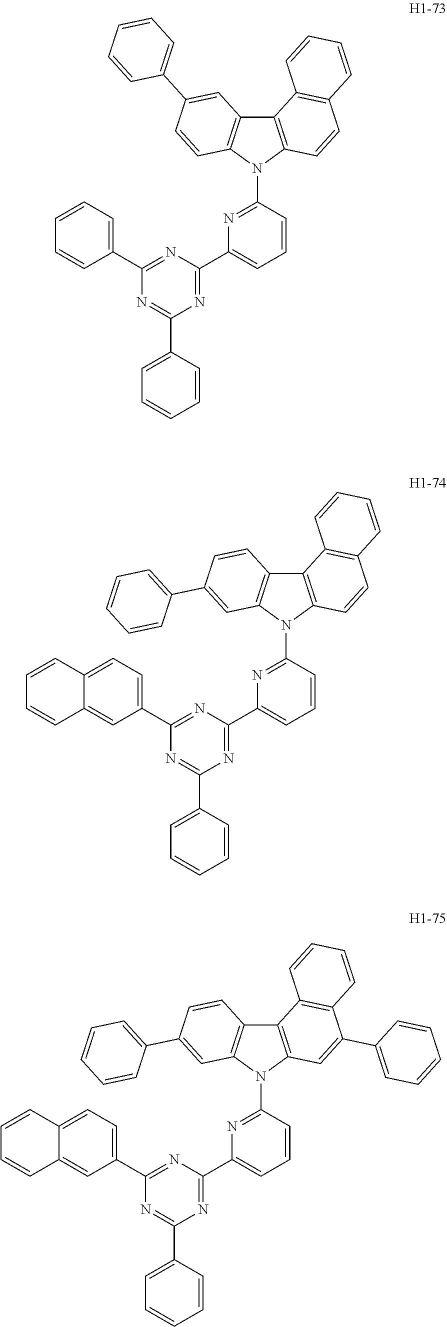 [SMILES: c1ccc(-c2ccc3c(c2)c2c4ccccc4ccc2n3-c2cccc(-c3nc(-c4ccccc4)nc(-c4ccccc4)n3)n2)cc1 ; c1ccc(-c2ccc3c4c5ccccc5ccc4n(-c4cccc(-c5nc(-c6ccccc6)nc(-c6ccc7ccccc7c6)n5)n4)c3c2)cc1 ; c1ccc(-c2ccc3c4c5ccccc5c(-c5ccccc5)cc4n(-c4cccc(-c5nc(-c6ccccc6)nc(-c6ccc7ccccc7c6)n5)n4)c3c2)cc1]

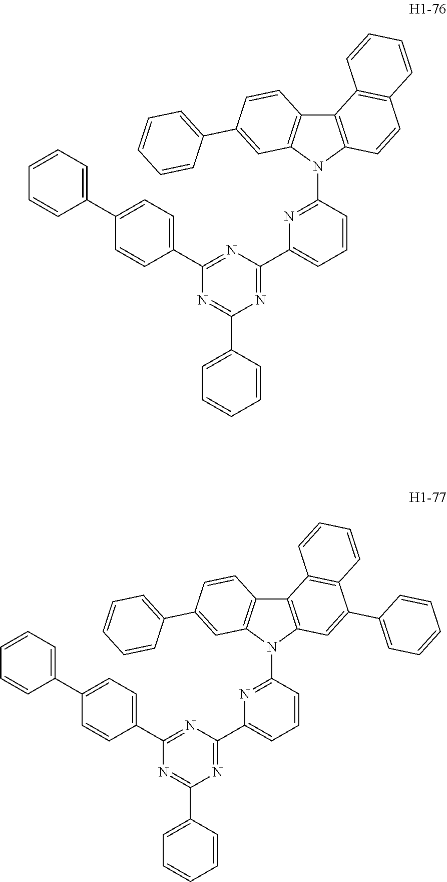 [SMILES: c1ccc(-c2ccc(-c3nc(-c4ccccc4)nc(-c4cccc(-n5c6cc(-c7ccccc7)ccc6c6c7ccccc7ccc65)n4)n3)cc2)cc1 ; c1ccc(-c2ccc(-c3nc(-c4ccccc4)nc(-c4cccc(-n5c6cc(-c7ccccc7)ccc6c6c7ccccc7c(-c7ccccc7)cc65)n4)n3)cc2)cc1]

According to one embodiment, the present disclosure provides an organic electroluminescent material comprising the organic electroluminescent compound of formula 1, and an organic electroluminescent device comprising the material.

The material may consist of the organic electroluminescent compound alone of the present disclosure. Otherwise, the material may be a mixture or a composition that further comprises a conventional compound(s) which has been comprised in an organic electroluminescent material, in addition to the compound of the present disclosure.

The organic electroluminescent device of the present disclosure may comprise a first electrode, a second electrode, and at least one organic layer disposed between the first and second electrodes. The organic layer may comprise at least one organic electroluminescent compound of formula 1.

One of the first and second electrodes may be an anode, and the other may be a cathode. The organic layer may comprise a light-emitting layer, and may further comprise at least one layer selected from a hole injection layer, a hole transport layer, a hole auxiliary layer, an auxiliary light-emitting layer, an electron transport layer, an electron buffering layer, an electron injection layer, an interlayer, a hole blocking layer, and an electron blocking layer, wherein the hole auxiliary layer or the auxiliary light-emitting layer is interposed between the hole transport layer and the light-emitting layer, and modulates hole mobility. The hole auxiliary layer or the auxiliary light-emitting layer has the effects to provide improved efficiency and lifespan of the organic electroluminescent device.

According to one embodiment of the present disclosure, the compound of formula 1 of the present disclosure may be comprised in the light-emitting layer as a host material. Preferably, the light-emitting layer may further comprise at least one dopant, and if needed, a compound other than the organic electroluminescent compound of formula 1 of the present disclosure may be comprised additionally as a second host material. The weight ratio between the first host material and the second host material is in the range of 1:99 to 99:1. It is preferable that a doping amount of the dopant compound is less than 20 wt % based on the total amount of the host compound and the dopant compound.

The second host material may be from any of the known phosphorescent host materials. Preferably, the second host material may be selected from the group consisting of the phosphorescent hosts of formula 7 below.

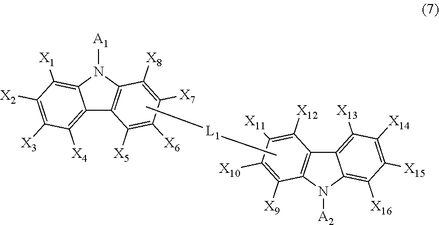

wherein
A₁ and A₂, each independently, represent a substituted or unsubstituted (C6-C30)aryl;
L₁ represents a single bond or a substituted or unsubstituted (C6-C30)arylene;
X₁ to X₁₆, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3 to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino; or may be linked to an adjacent substituent(s) to form a substituted or unsubstituted (C3-C30), mono- or polycyclic, alicyclic or aromatic ring, whose carbon atom(s) may be replaced with at least one heteroatom selected from nitrogen, oxygen, and sulfur.

The compound of formula 7 of the present disclosure may be represented by any one of the following formulae 8 to 11:

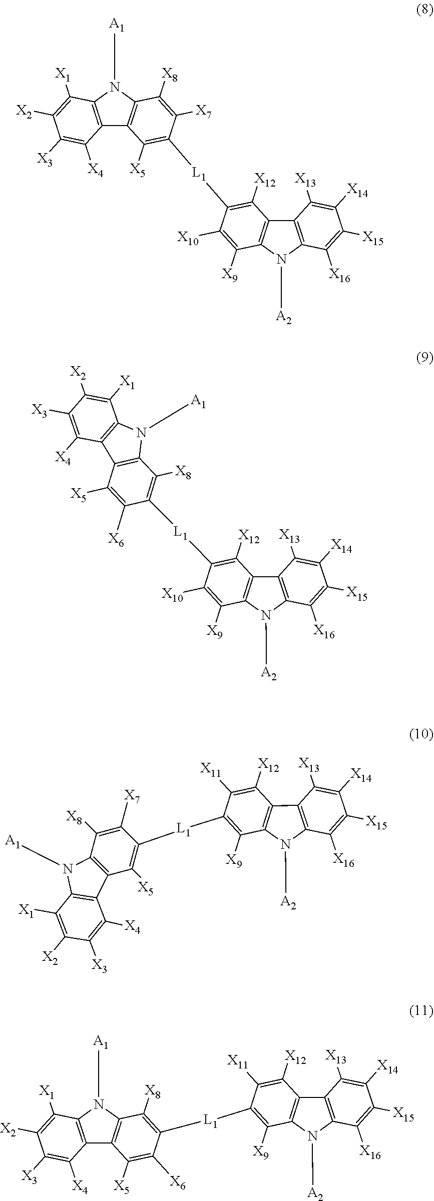

wherein A₁, A₂, L₁ and X₁ to X₁₆ are as defined in formula 7 above.

In formula 7, A₁ and A₂, each independently, may represent a substituted or unsubstituted (C6-C30)aryl; preferably, each independently, may represent a substituted or unsubstituted (C6-C18)aryl; and more preferably, each independently, may represent a (C6-C18)aryl substituted or unsubstituted with a (C1-C6)alkyl, a (C6-C18)aryl, a (5 to 20-membered)heteroaryl or a tri(C6-C12)arylsilyl. Specifically, A₁ and A₂, each independently, may be selected from the group consisting of a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted benzofluorenyl, a substituted or unsubstituted phenanthrenyl, a substituted or unsubstituted anthracenyl, a substituted or unsubstituted indenyl, a substituted or unsubstituted triphenylenyl, a substituted or unsubstituted pyrenyl, a substituted or unsubstituted tetracenyl, a substituted or unsubstituted perylenyl, a substituted or unsubstituted chrysenyl, a substituted or unsubstituted phenylnaphthyl, a substituted or unsubstituted naphthylphenyl, and a substituted or unsubstituted fluoranthenyl.

In formula 7, L₁ may represent a single bond or a substituted or unsubstituted (C6-C30)arylene; preferably a single bond or a substituted or unsubstituted (C6-C18)arylene; and more preferably a single bond or an unsubstituted (C6-C18)arylene. Specifically, L₁ may represent a single bond, a substituted or unsubstituted phenylene, a substituted or unsubstituted naphthylene, or a substituted or unsubstituted biphenylene.

More specifically, L₁ may represent a single bond or may be represented by any one of the following formulae 12 to 24.

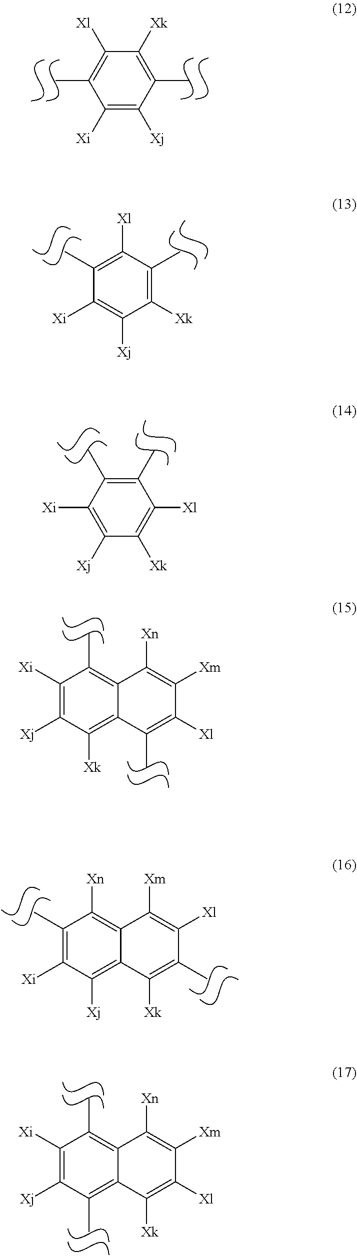

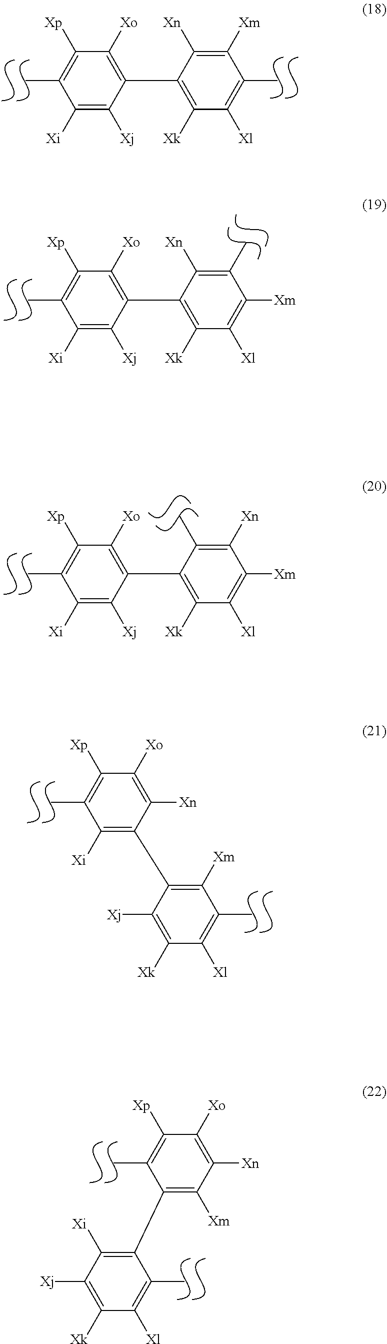

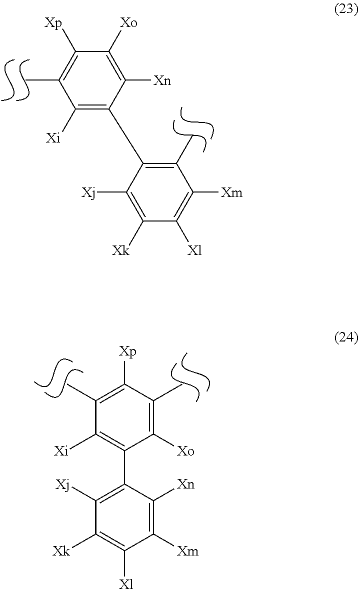

wherein,
Xi to Xp, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3 to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted mono- or di-(C6-C30)arylamino; or may be linked to an adjacent substituent(s) to form a substituted or unsubstituted (C3-C30), mono- or polycyclic, alicyclic or aromatic ring, whose carbon atom(s) may be replaced with at least one heteroatom selected from the group consisting of nitrogen, oxygen, and sulfur; and represents a bonding site.

Preferably, Xi to Xp, each independently, may represent hydrogen, a halogen, a cyano, a (C1-C10)alkyl, a (C3-C20)cycloalkyl, a (C6-C12)aryl, a (C1-C6)alkyldi(C6-C12)arylsilyl, or a tri(C6-C12)arylsilyl; and more preferably, each independently, may represent hydrogen, a cyano, a (C1-C6)alkyl, or a tri(C6-C12)arylsilyl.

In formula 7, X₁ to X₁₆, each independently, may represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3 to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino; or may be linked to an adjacent substituent(s) to form a substituted or unsubstituted (C3-C30), mono- or polycyclic, alicyclic or aromatic ring, whose carbon atom(s) may be replaced with at least one heteroatom selected from the group consisting of nitrogen, oxygen, and sulfur; perferably, each independently, may represent hydrogen, or a substituted or unsubstituted (5- to 20-membered) heteroaryl, or may be linked to an adjacent substituent(s) to form a substituted or unsubstituted (C6-C12), mono- or polycyclic, alicyclic or aromatic ring; and more perferably, each independently, may represent hydrogen, or an unsubstituted (5- to 20-membered) heteroaryl, or may be linked to an adjacent substituent(s) to form a substituted or unsubstituted (C6-C12), mono- or polycyclic aromatic ring.

Organic electroluminescent compounds of formula 7 of the present disclosure include the following, but are not limited thereto:

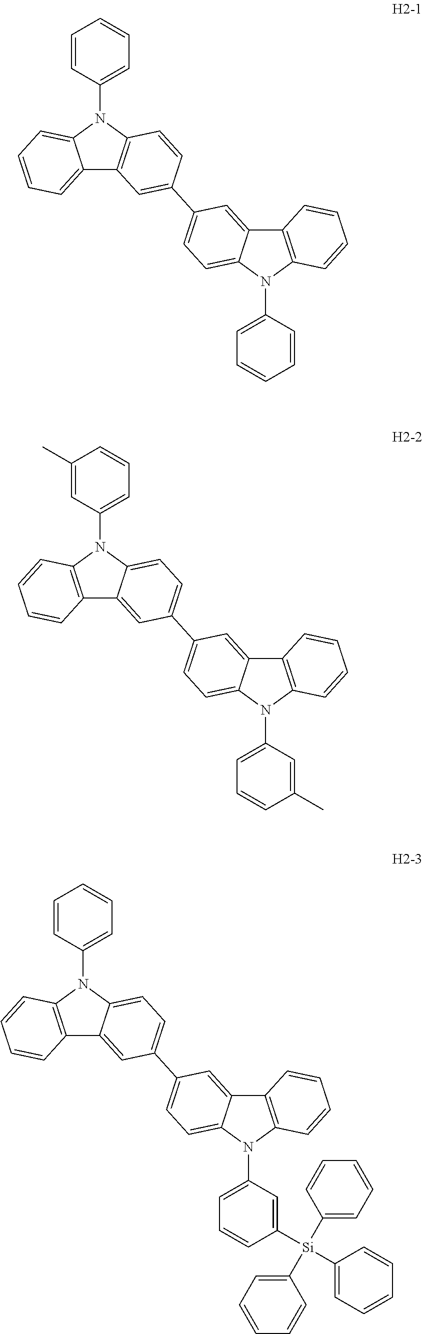 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

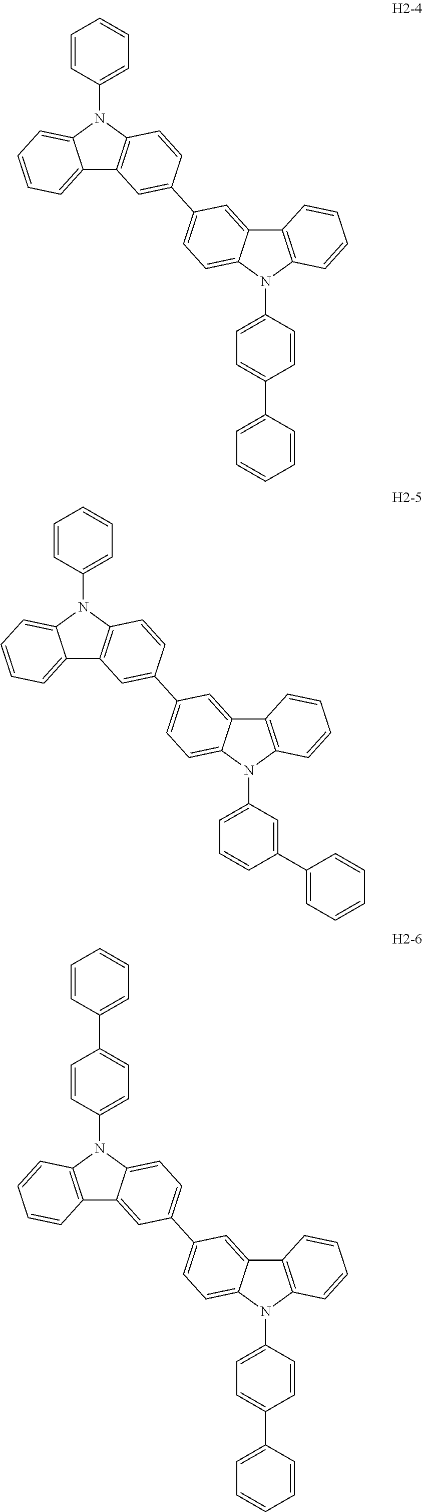 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

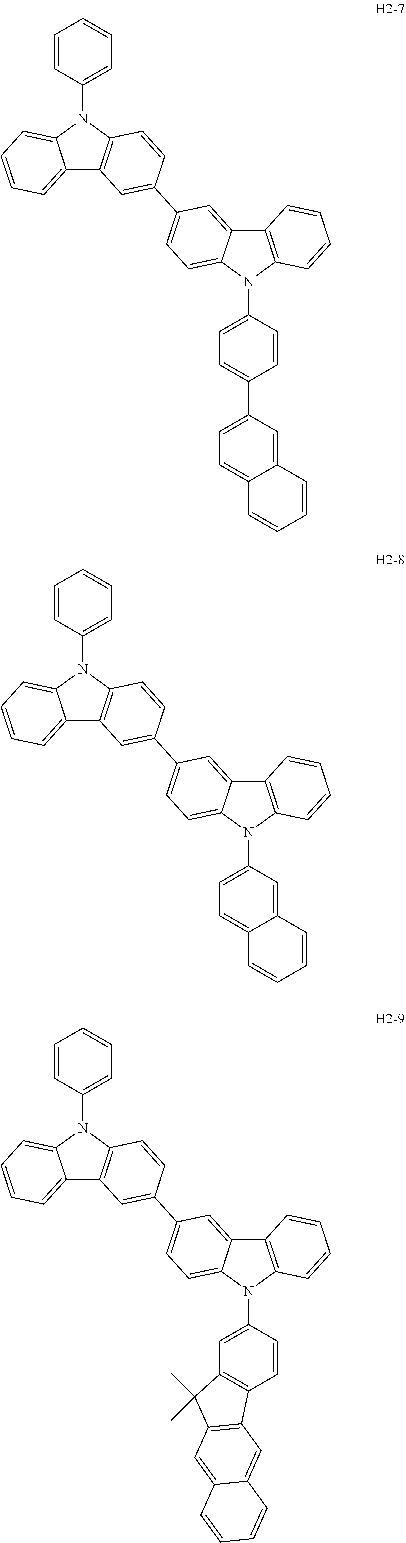 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21]

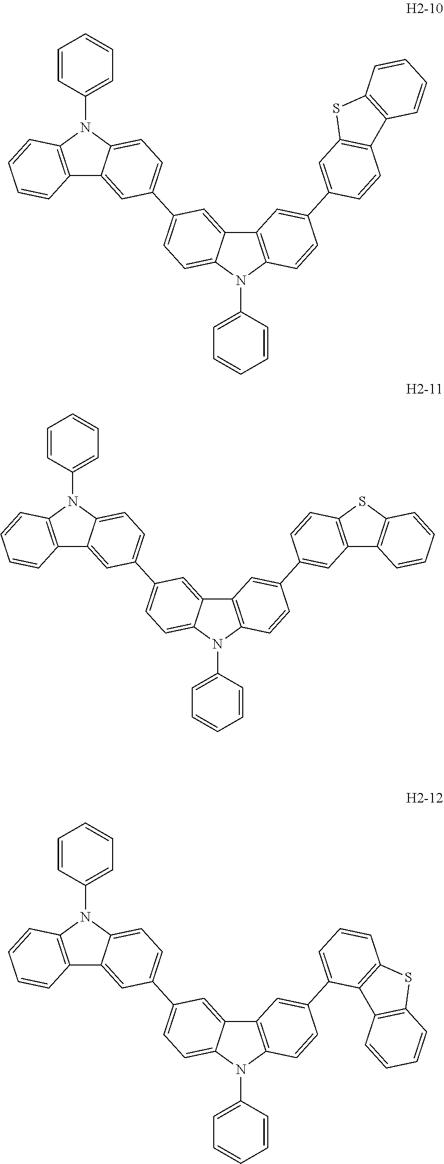 [SMILES: c1ccc(-n2c3ccccc3c3cc(-c4ccc5c(c4)c4cc(-c6ccc7c(c6)sc6ccccc67)ccc4n5-c4ccccc4)ccc32)cc1 ; c1ccc(-n2c3ccccc3c3cc(-c4ccc5c(c4)c4cc(-c6ccc7sc8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1]

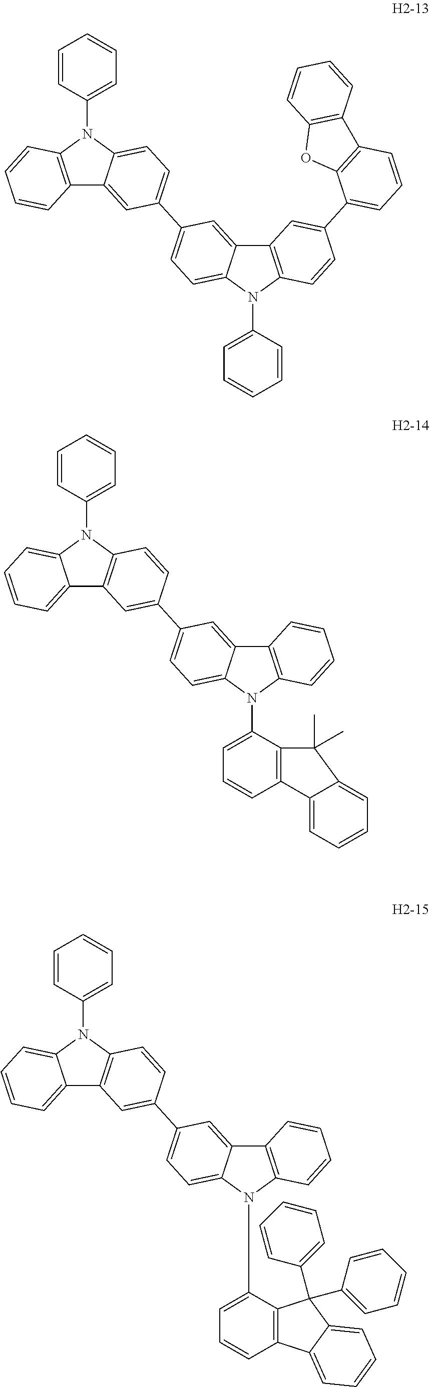 [SMILES: c1ccc(-n2c3ccccc3c3cc(-c4ccc5c(c4)c4cc(-c6cccc7c6oc6ccccc67)ccc4n5-c4ccccc4)ccc32)cc1 ; CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1]

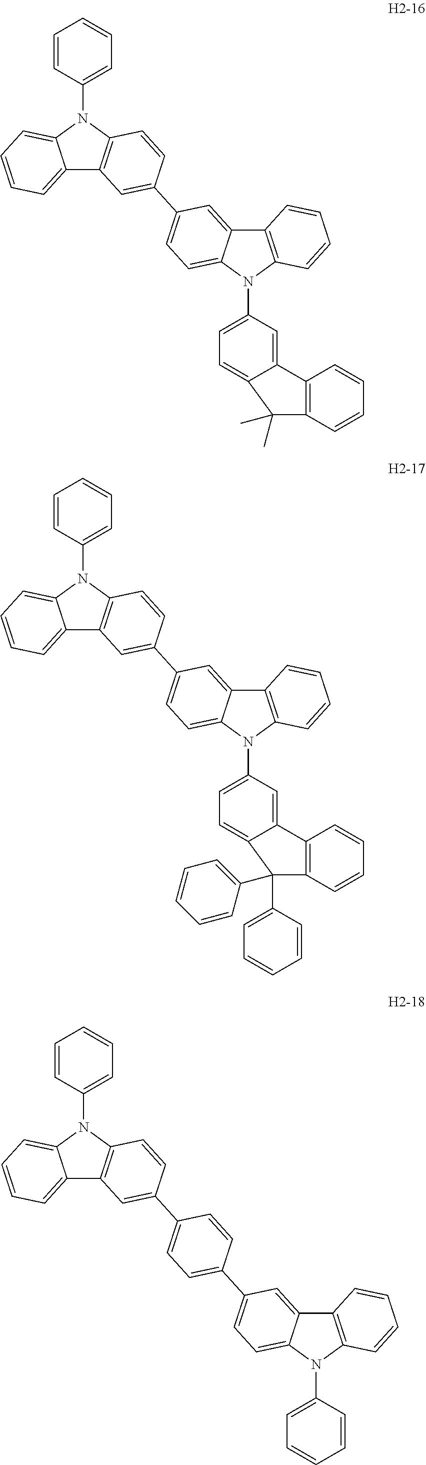 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1 ; c1ccc(-n2c3ccccc3c3cc(-c4ccc(-c5ccc6c(c5)c5ccccc5n6-c5ccccc5)cc4)ccc32)cc1]

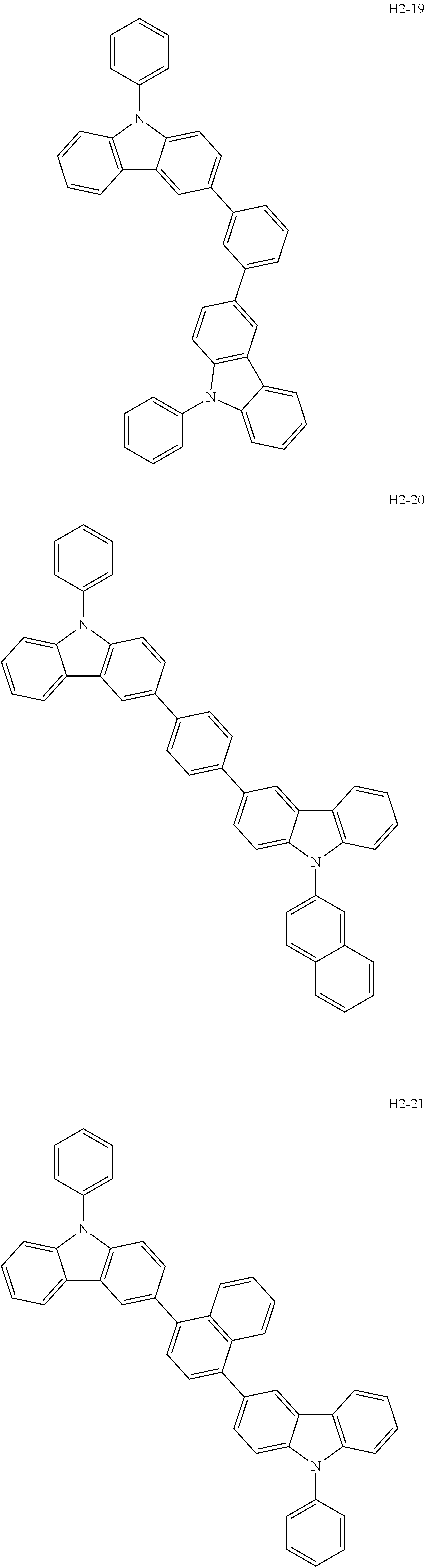 [SMILES: c1ccc(-n2c3ccccc3c3cc(-c4cccc(-c5ccc6c(c5)c5ccccc5n6-c5ccccc5)c4)ccc32)cc1 ; c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4ccc(-c5ccc6c(c5)c5ccccc5n6-c5ccccc5)c5ccccc45)ccc32)cc1]

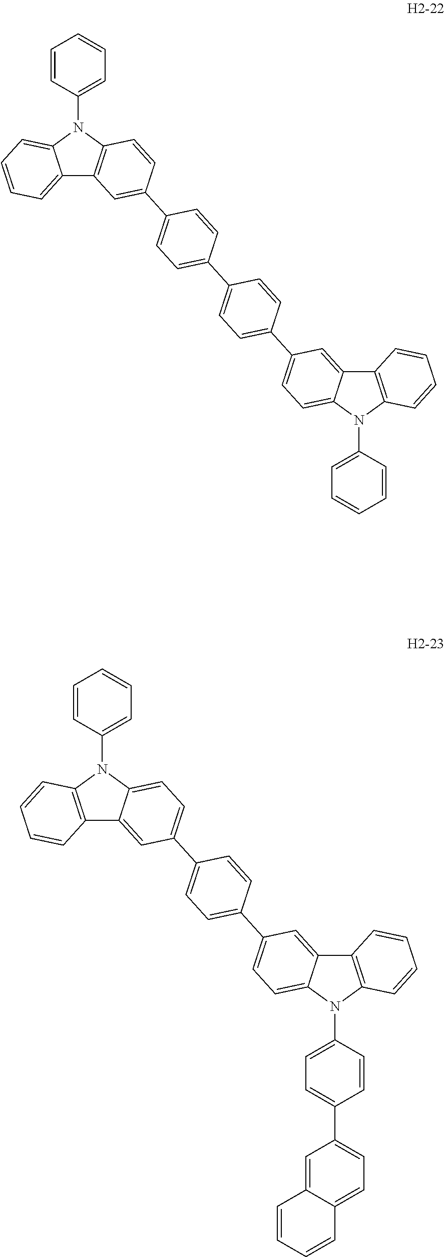 [SMILES: c1ccc(-n2c3ccccc3c3cc(-c4ccc(-c5ccc(-c6ccc7c(c6)c6ccccc6n7-c6ccccc6)cc5)cc4)ccc32)cc1 ; c1ccc(-n2c3ccccc3c3cc(-c4ccc(-c5ccc6c(c5)c5ccccc5n6-c5ccc(-c6ccc7ccccc7c6)cc5)cc4)ccc32)cc1]

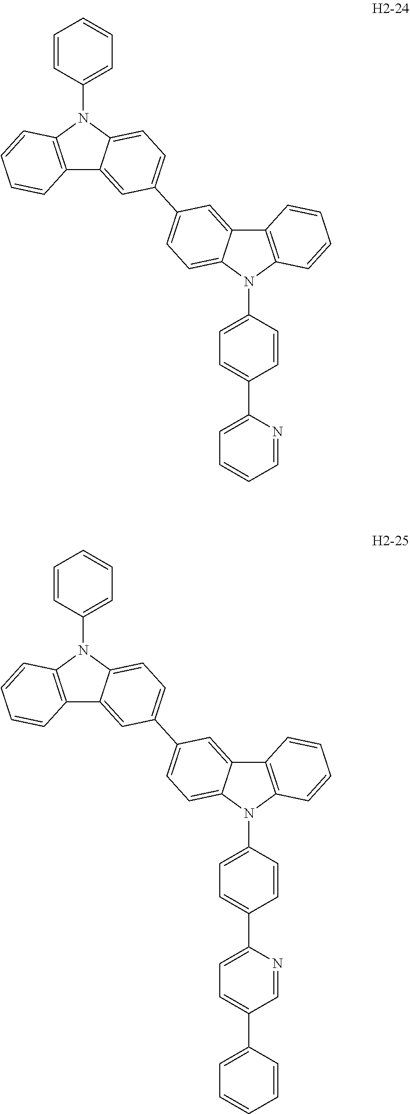 [SMILES: c1ccc(-n2c3ccccc3c3cc(-c4ccc5c(c4)c4ccccc4n5-c4ccc(-c5ccccn5)cc4)ccc32)cc1 ; c1ccc(-c2ccc(-c3ccc(-n4c5ccccc5c5cc(-c6ccc7c(c6)c6ccccc6n7-c6ccccc6)ccc54)cc3)nc2)cc1]

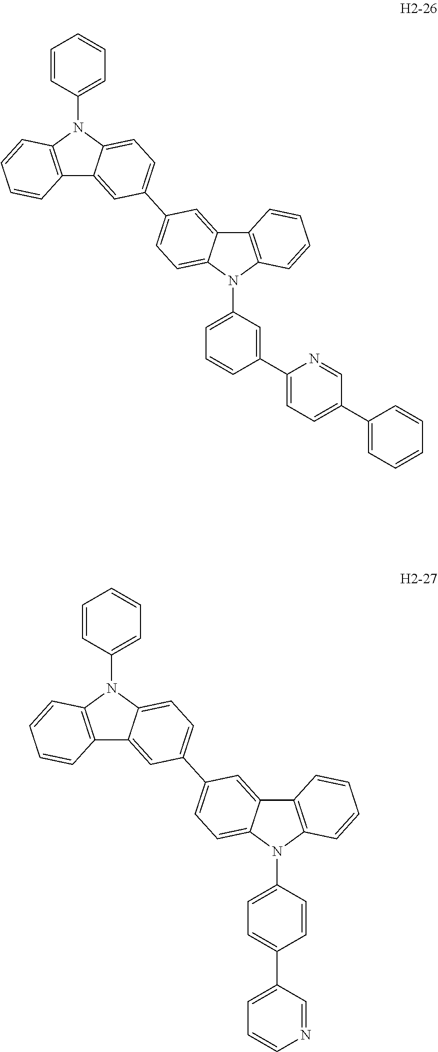 [SMILES: c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1]

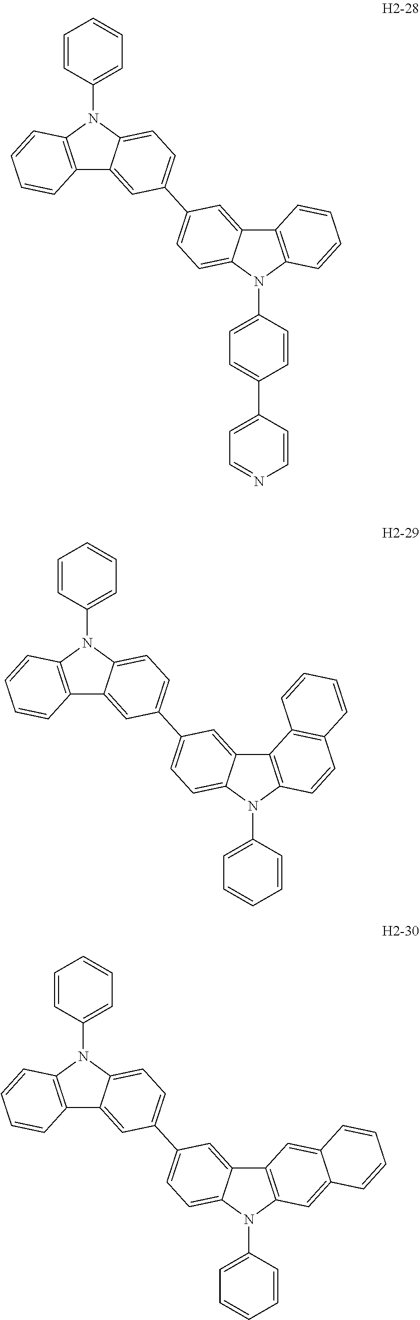 [SMILES: c1ccc(-n2c3ccccc3c3cc(-c4ccc5c(c4)c4ccccc4n5-c4ccc(-c5ccncc5)cc4)ccc32)cc1 ; c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1]

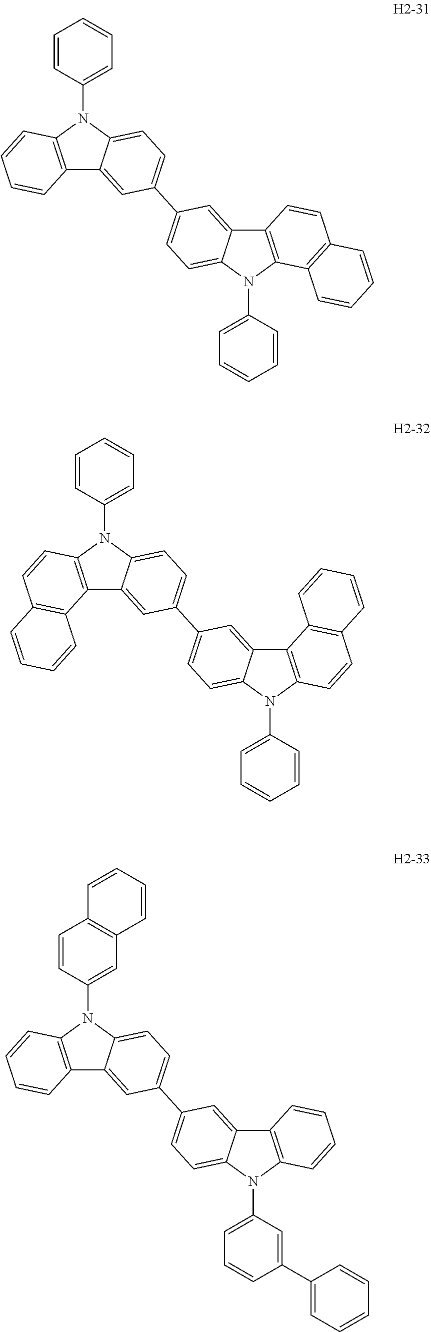 [SMILES: c1ccc(-n2c3ccccc3c3cc(-c4ccc5c(c4)c4ccc6ccccc6c4n5-c4ccccc4)ccc32)cc1 ; c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1]

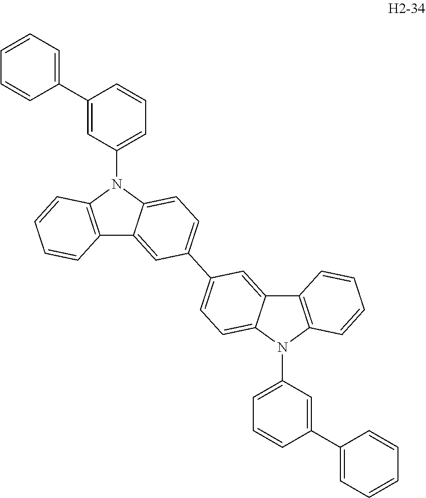 [SMILES: c1ccc(-c2cccc(-n3c4ccccc4c4cc(-c5ccc6c(c5)c5ccccc5n6-c5cccc(-c6ccccc6)c5)ccc43)c2)cc1]

The dopant to be comprised in the organic electroluminescent device of the present disclosure may be at least one phosphorescent dopant. The phosphorescent dopant material for the organic electroluminescent device of the present disclosure is not limited, but may be preferably selected from metallated complex compounds of iridium (Ir), osmium (Os), copper (Cu) or platinum (Pt), more preferably selected from ortho-metallated complex compounds of iridium (Ir), osmium (Os), copper (Cu) or platinum (Pt), and even more preferably ortho-metallated iridium complex compounds.

The compound selected from the following formulae 101 to 103 may be preferably used as the dopant to be comprised in the organic electroluminescent device of the present disclosure.

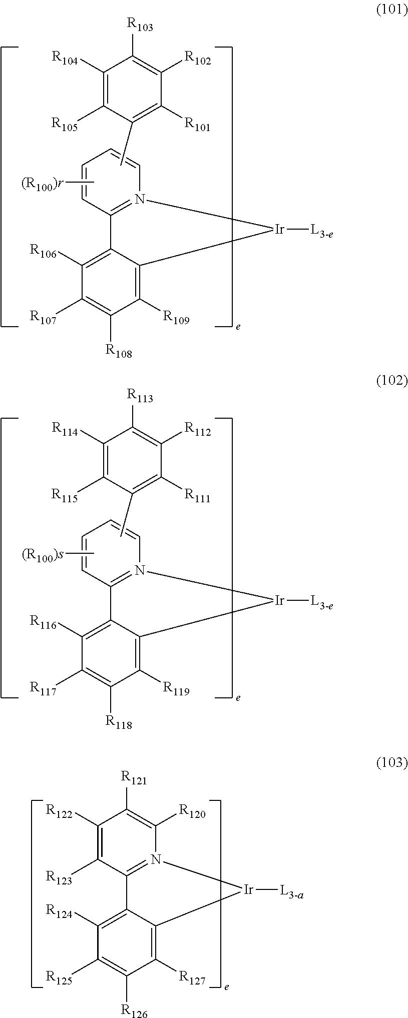

wherein L is selected from the following structures:

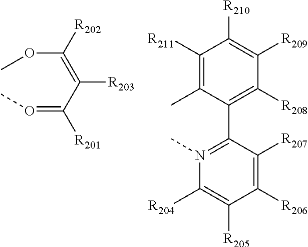

R₁₀₀ represents hydrogen, a substituted or unsubstituted (C1-C30)alkyl, or a substituted or unsubstituted (C3-C30)cycloalkyl;;
R₁₀₁ to R₁₀₉ and R₁₁₁ to R₁₂₃, each independently, represent hydrogen, deuterium, a halogen, a (C1-C30)alky substituted or unsubstituted with a halogen, a cyano, a substituted or unsubstituted (C1-C30)alkoxy, a substituted or unsubstituted (C6-C30)aryl, or a substituted or unsubstituted (C3-C30)cycloalkyl; R₁₀₆ to R₁₀₉, each independently, may be linked to an adjacent substituent(s) to form a substituted or unsubstituted fused ring, for example, a fluorene substituted or unsubstituted with an alkyl, a dibenzothiophene substituted or unsubstituted with an alkyl, or a dibenzofuran substituted or unsubstituted with an alkyl; R₁₂₀ to R₁₂₃, each independently, may be linked to an adjacent substituent(s) to form a substituted or unsubstituted fused ring, for example, a quinoline substituted or unsubstituted with a halogen, an alkyl or an aryl;
R₁₂₄ to R₁₂₇, each independently, represent hydrogen, deuterium, a halogen, a substituted or unsubstituted (C1-C30)alkyl, or a substituted or unsubstituted (C6-C30)aryl; R₁₂₄ to R₁₂₇, each independently, may be linked to an adjacent substituent(s) to form a substituted or unsubstituted fused ring, for example, a fluorene substituted or unsubstituted with an alkyl, a dibenzothiophene substituted or unsubstituted with an alkyl, or a dibenzofuran substituted or unsubstituted with an alkyl;
R₂₀₁ to R₂₁₁, each independently, represent hydrogen, deuterium, a halogen, a (C1-C30)alkyl substituted or unsubstituted with a halogen, a substituted or unsubstituted (C3-C30)cycloalkyl, or a substituted or unsubstituted (C6-C30)aryl; R₂₀₈ to R₂₁₁, each independently, may be linked to an adjacent substituent(s) to form a substituted or unsubstituted fused ring, for example, a fluorene substituted or unsubstituted with an alkyl, a dibenzothiophene substituted or unsubstituted with an alkyl, or a dibenzofuran substituted or unsubstituted with an alkyl;
r and s, each independently, represent an integer of 1 to 3; when r or s is an integer of 2 or more, each of R₁₀₀ may be the same or different; and
e represents an integer of 1 to 3.

Specifically, the phosphorescent dopant includes the following:

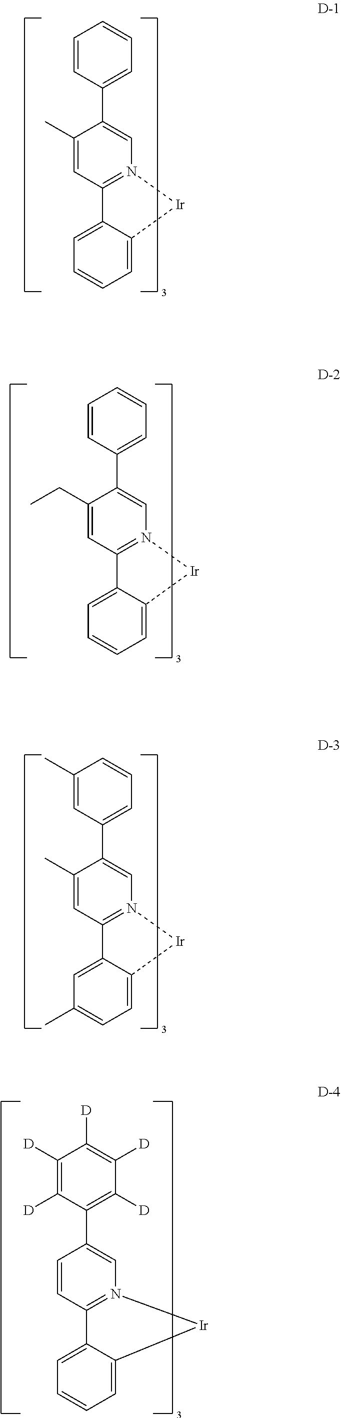

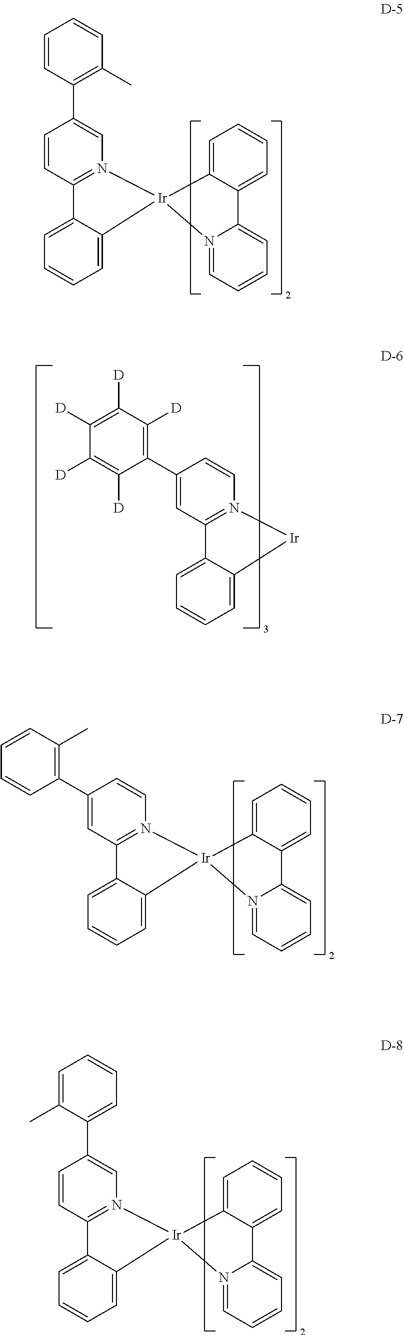

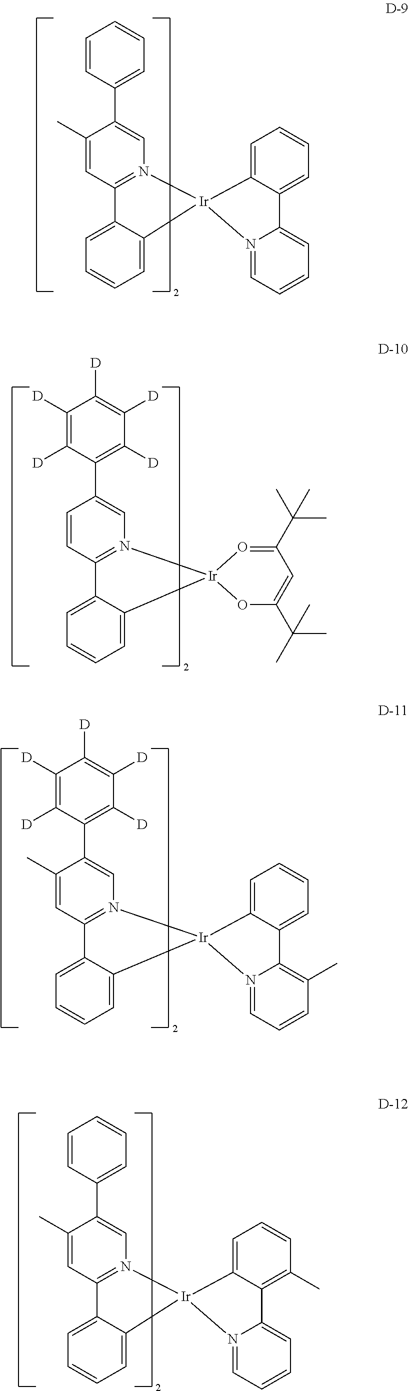

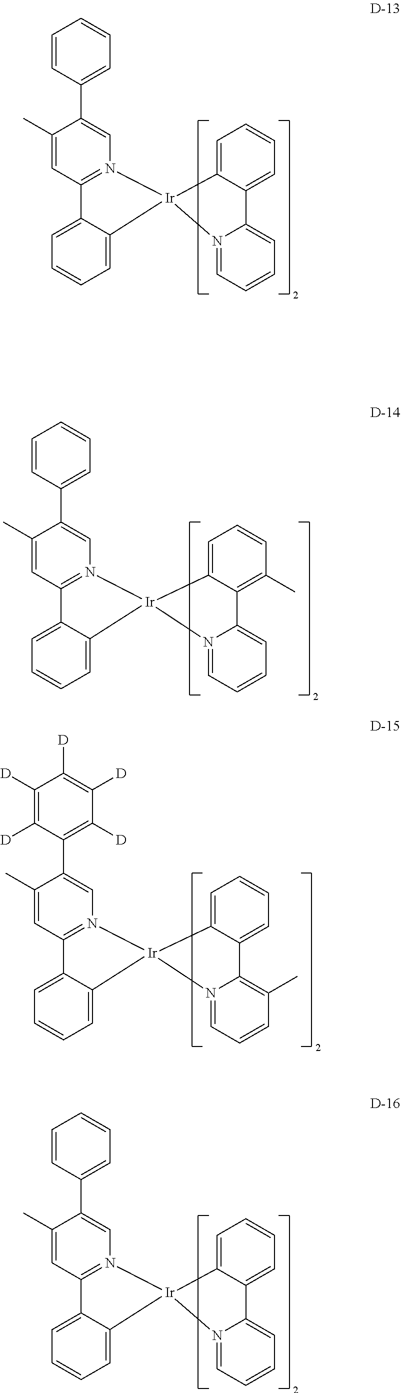

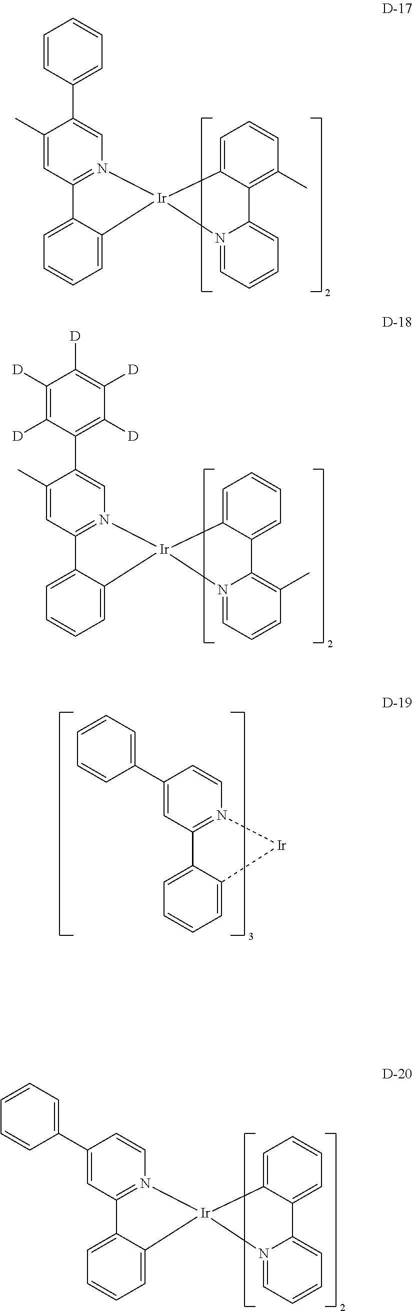

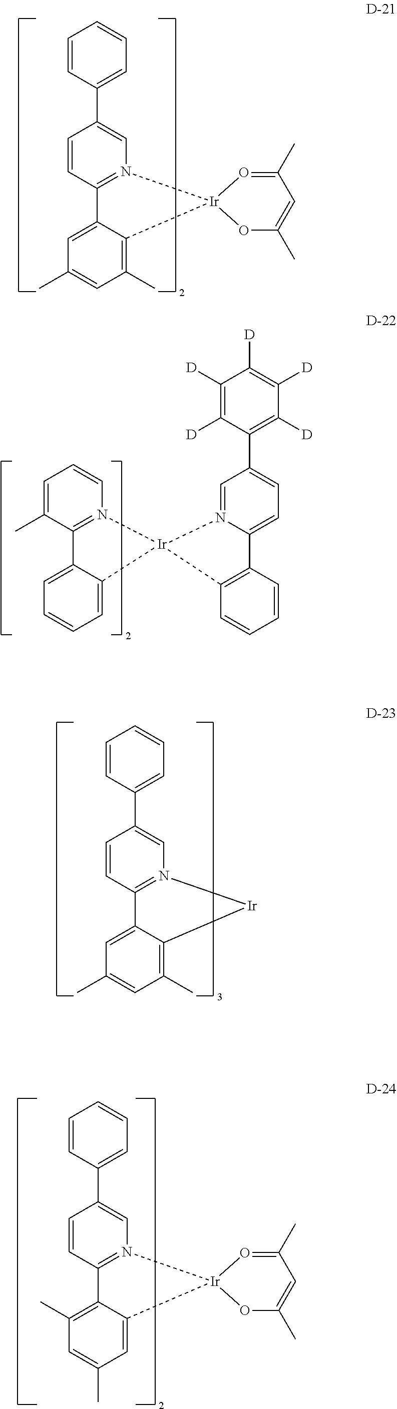

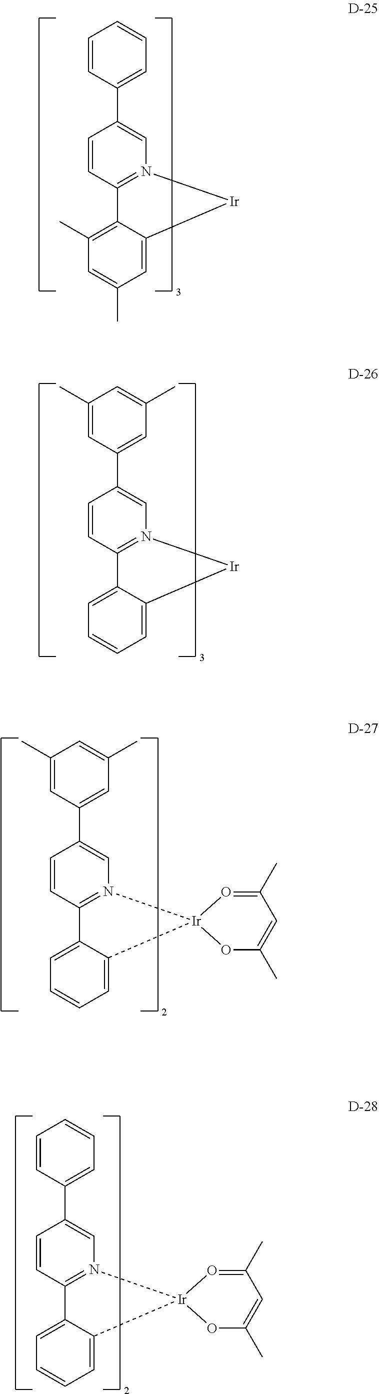

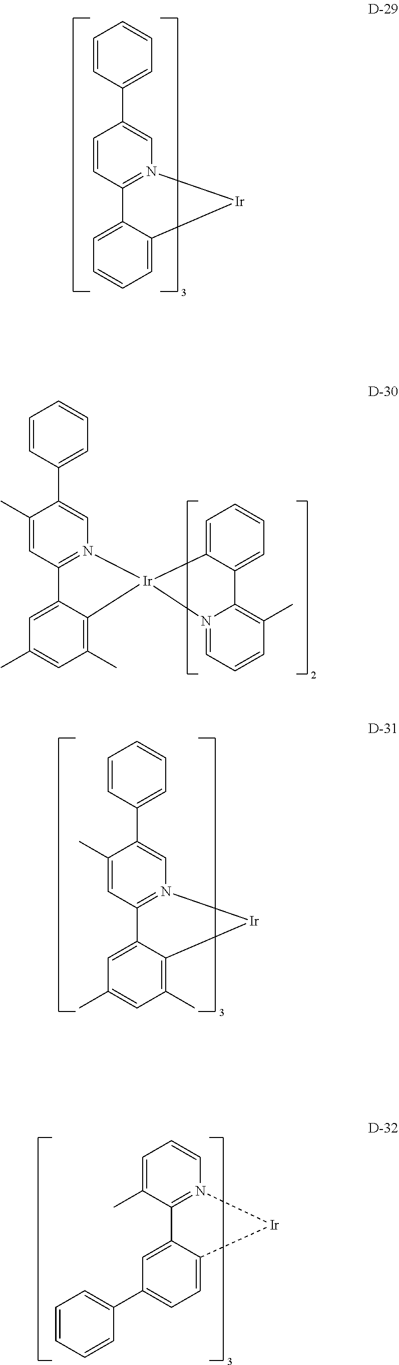

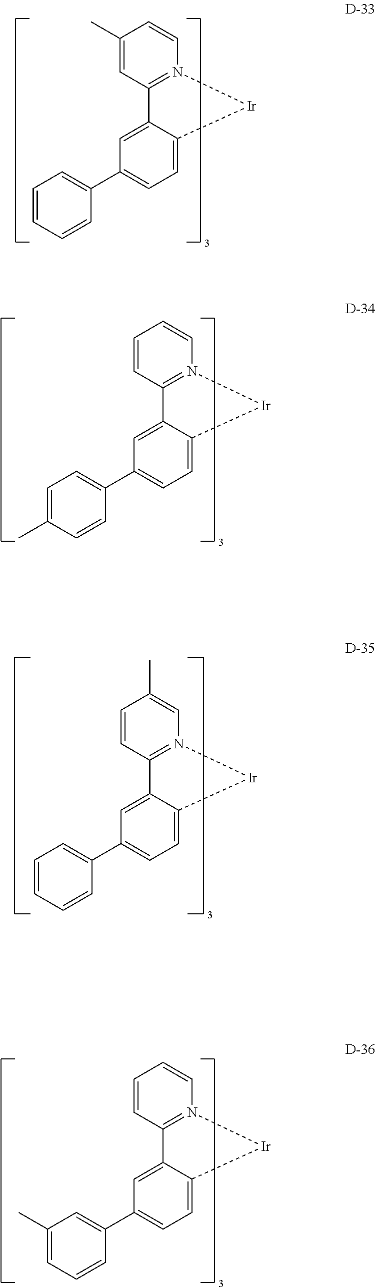

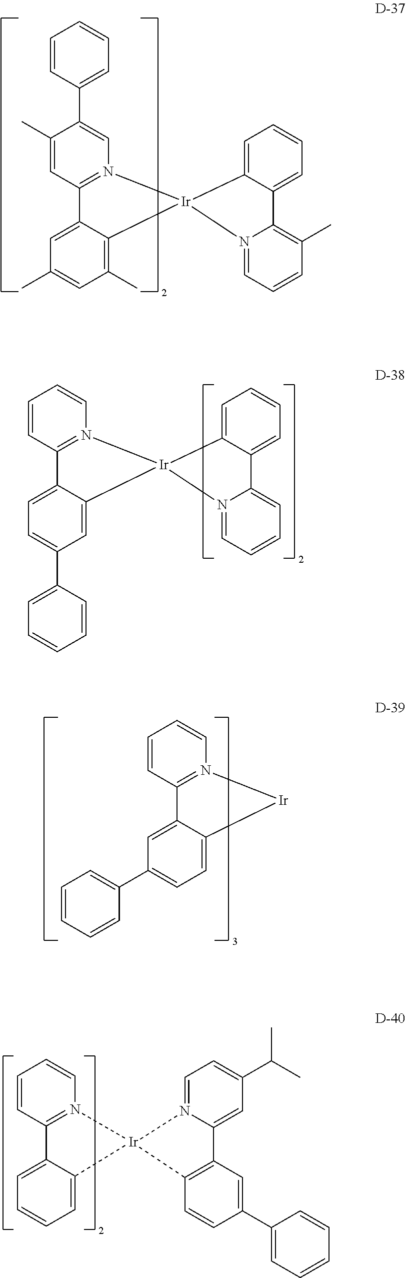

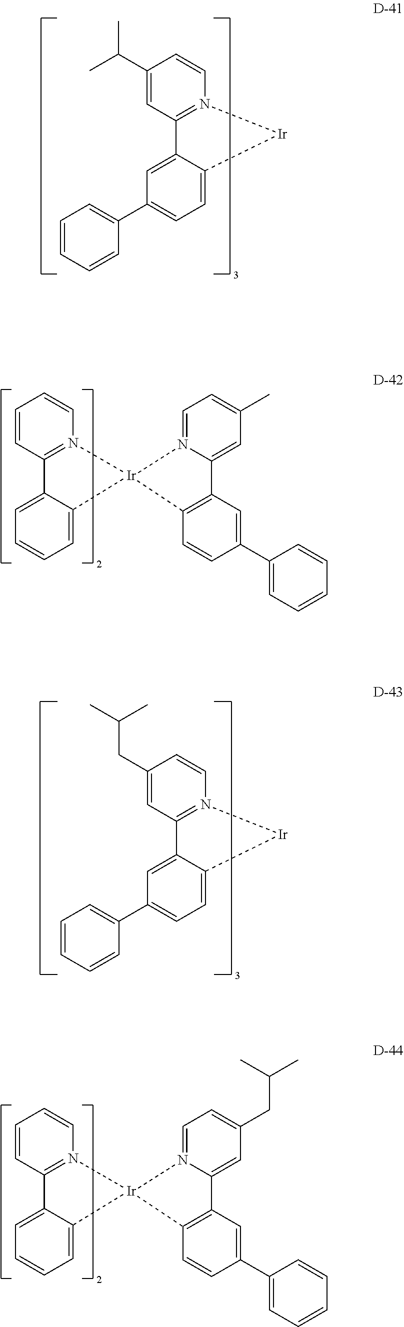

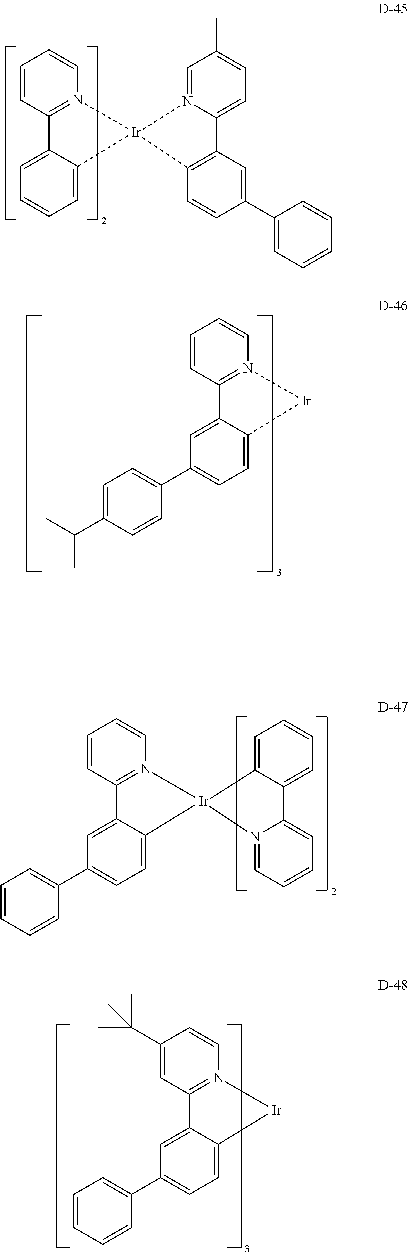

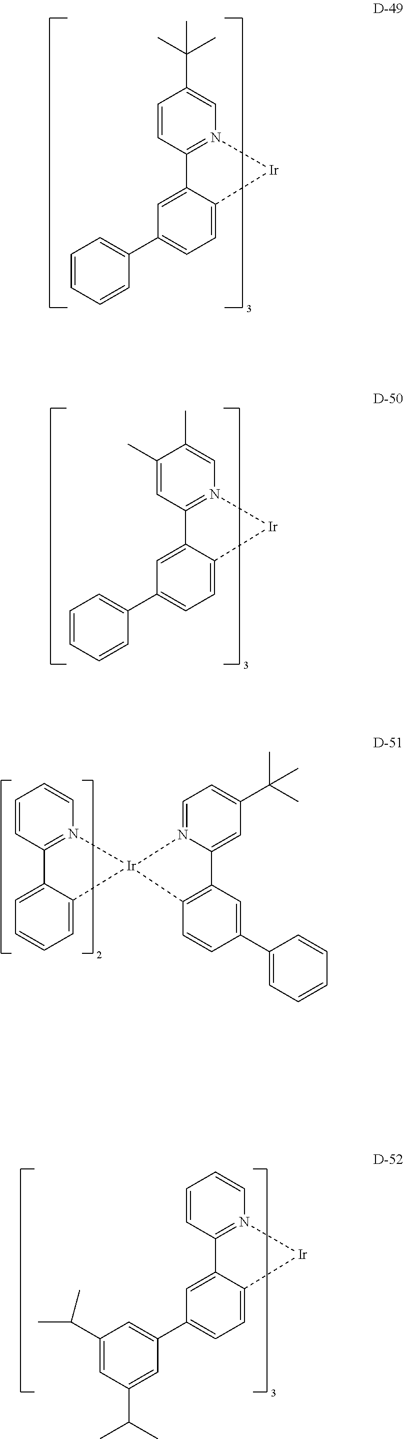

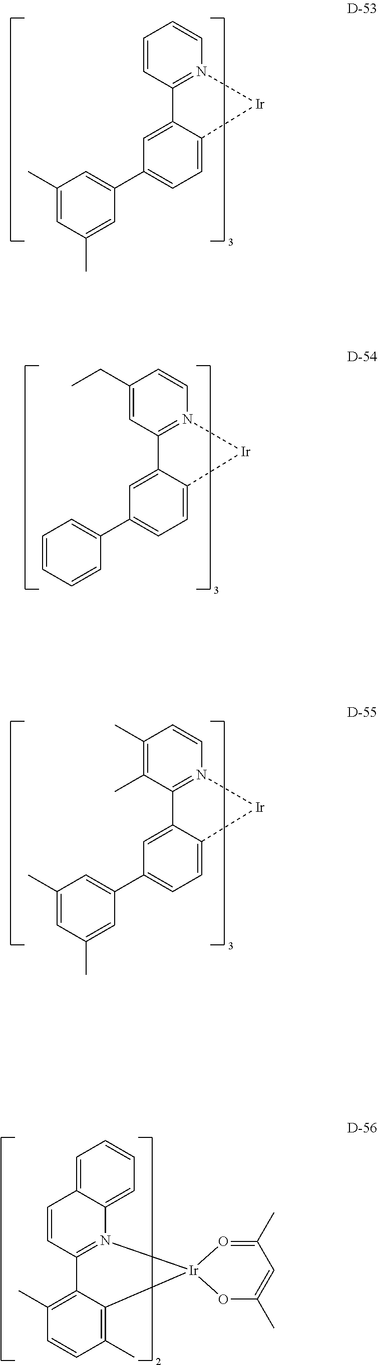

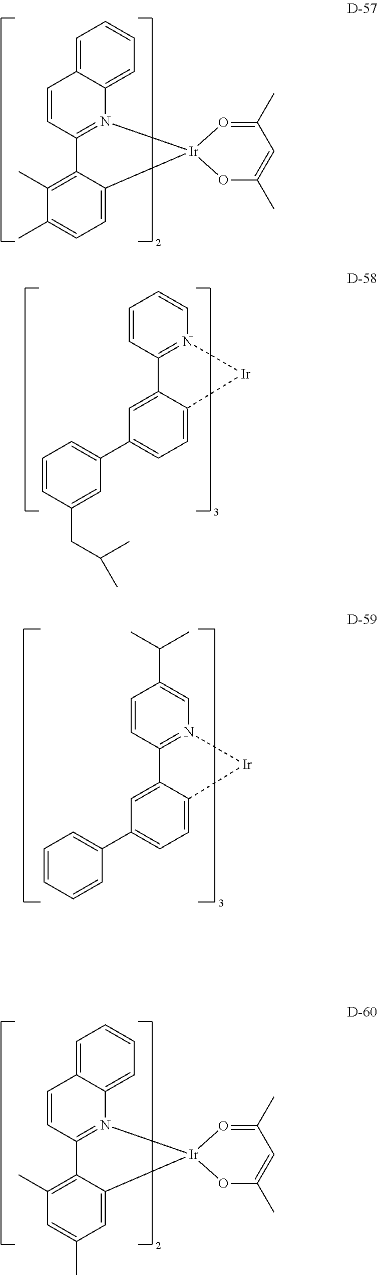

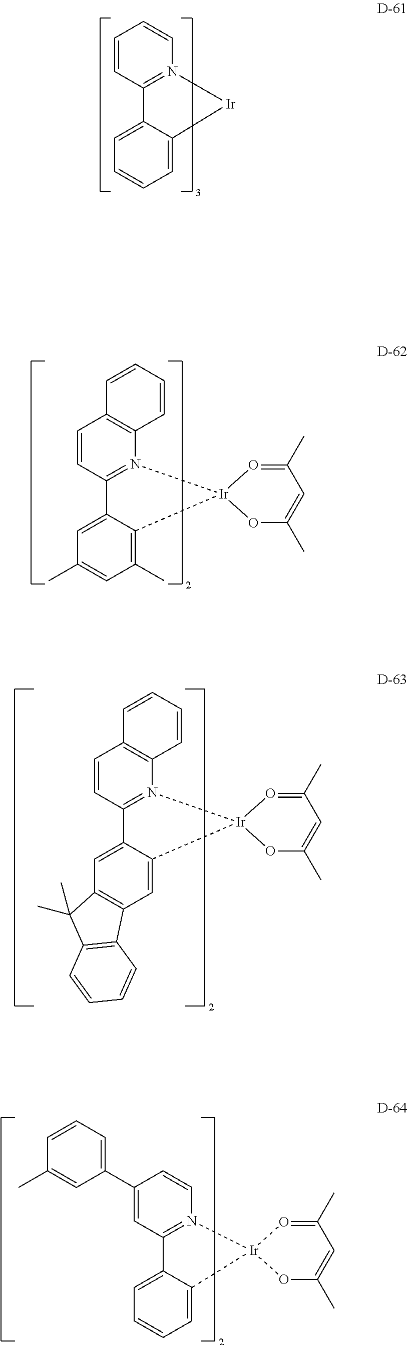

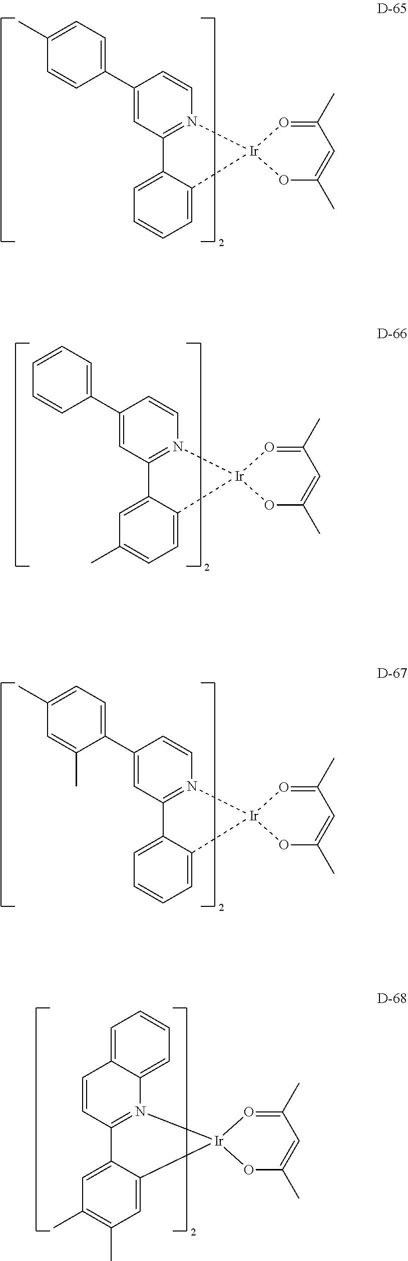

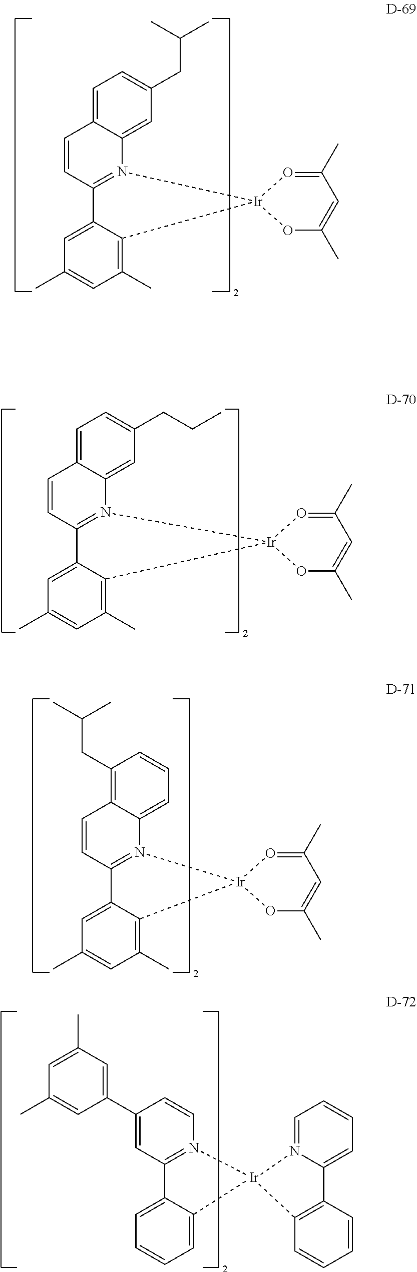

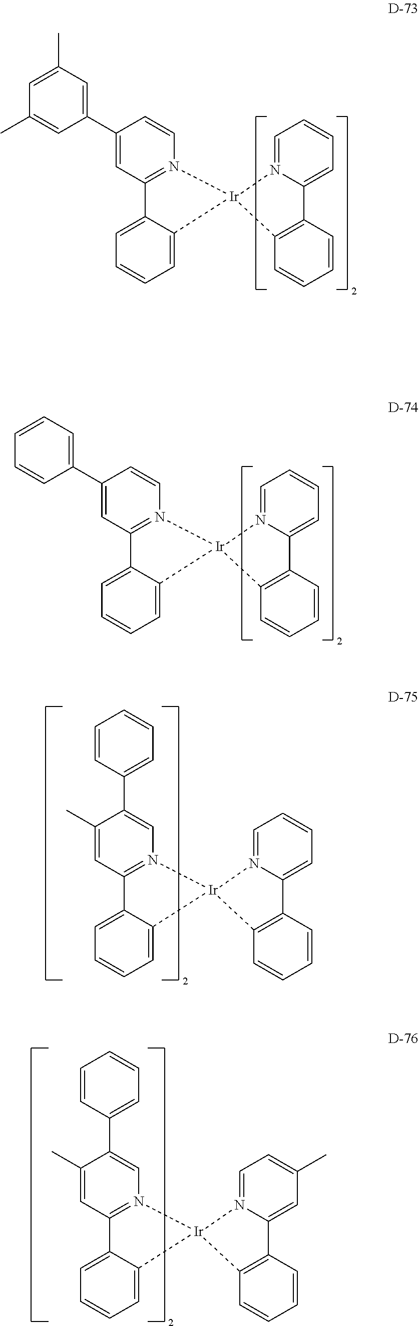

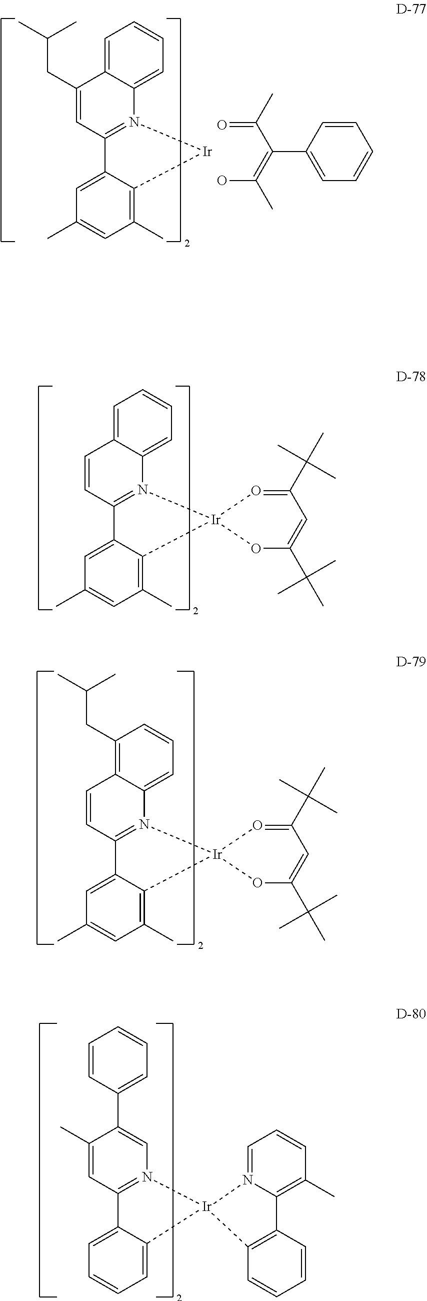

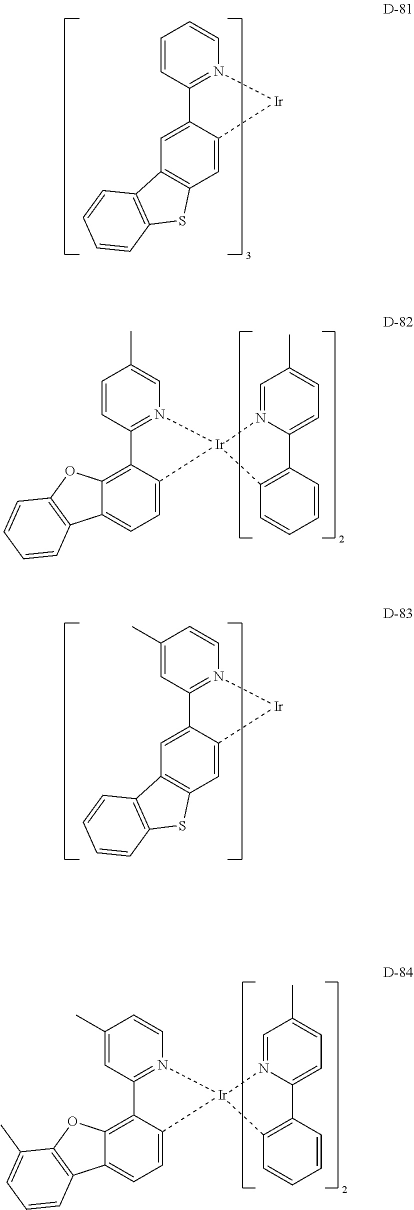

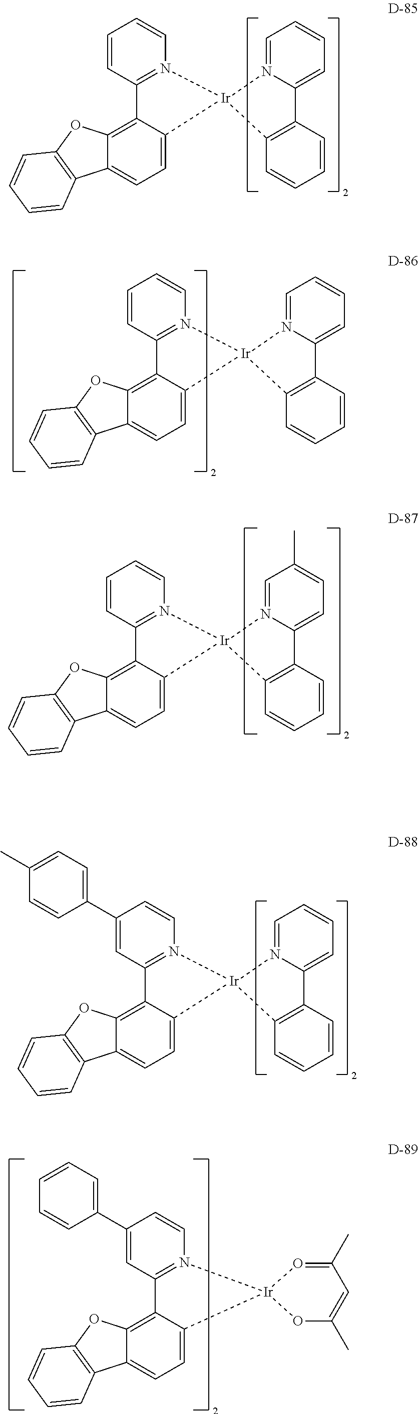

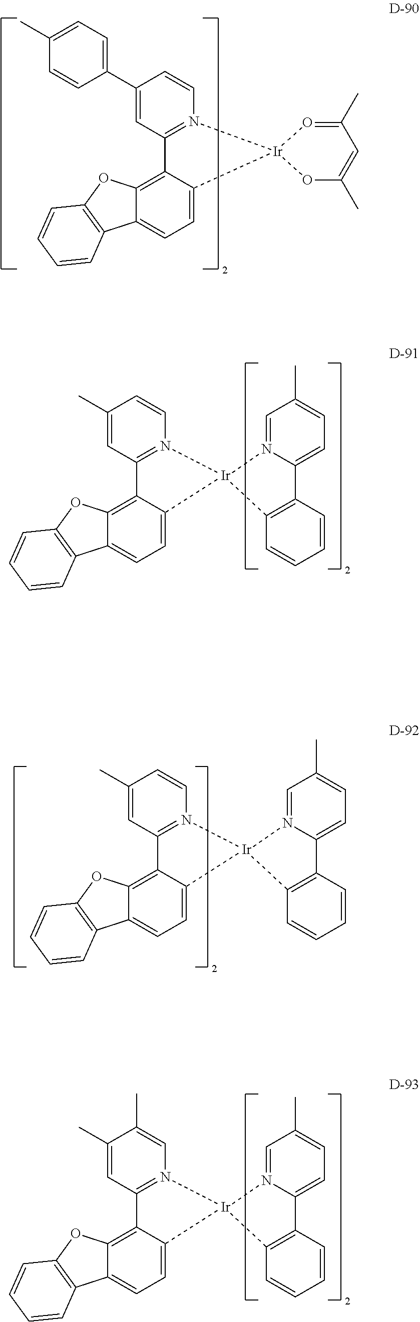

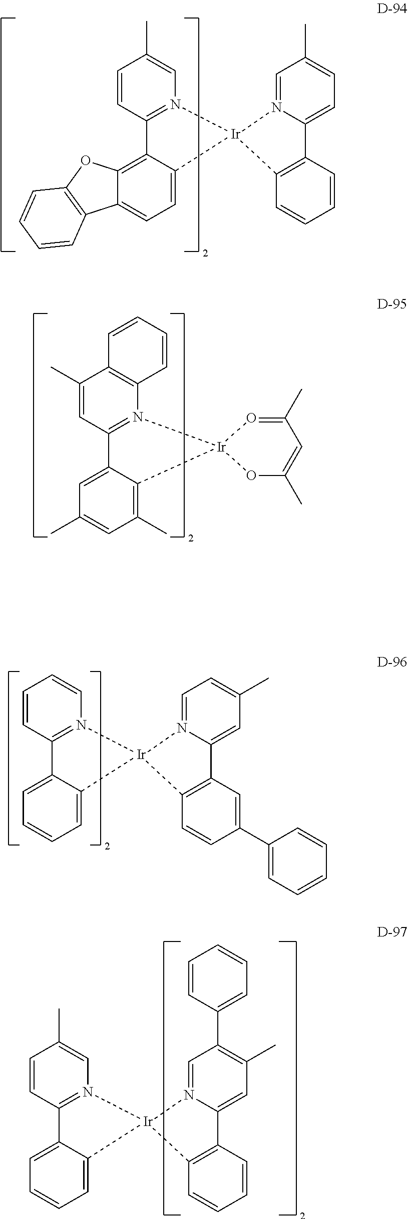

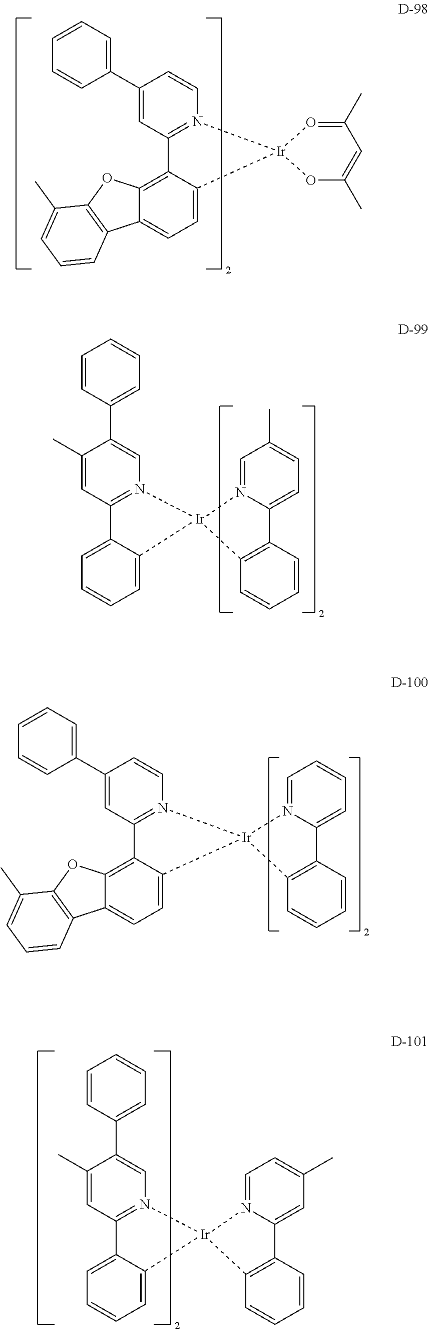

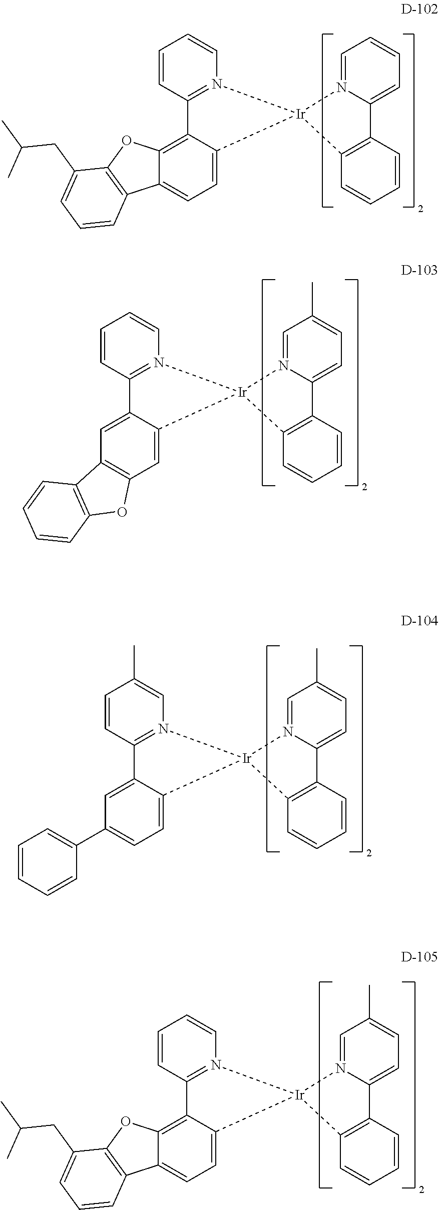

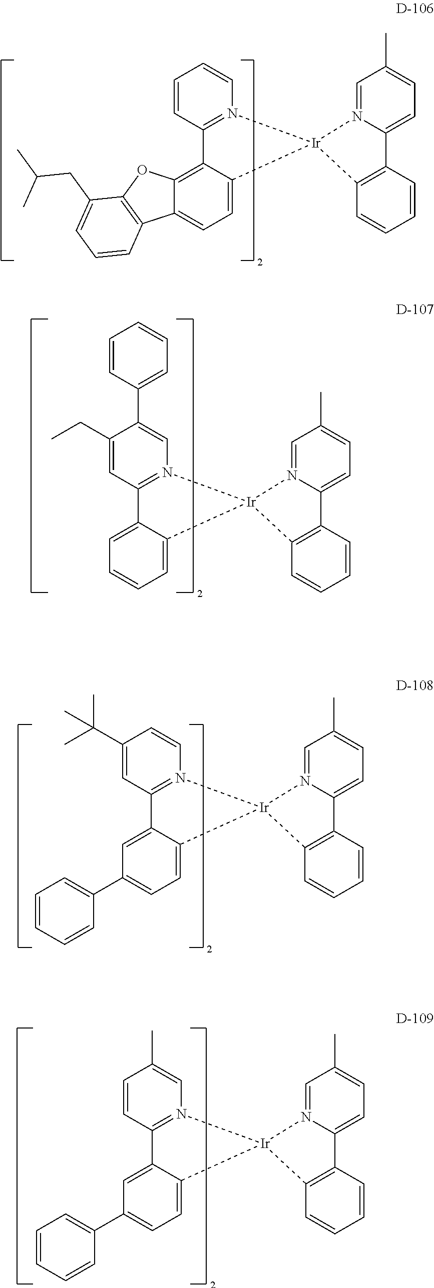

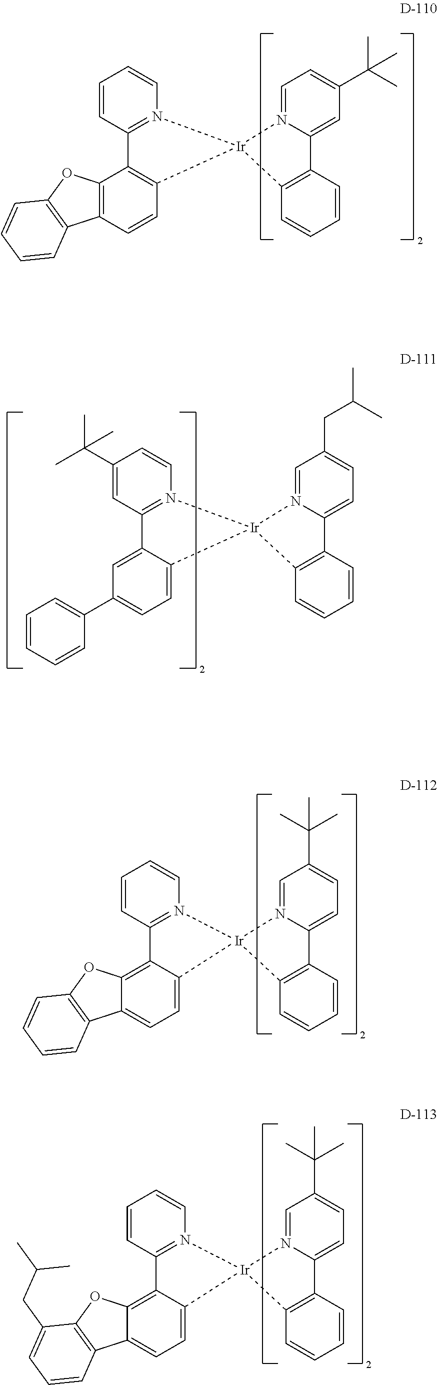

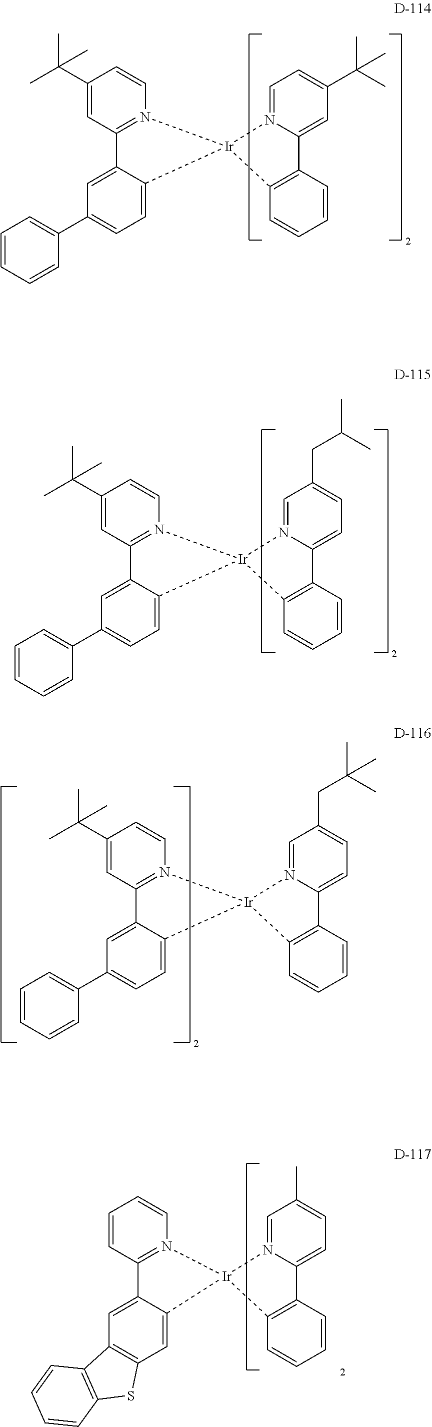

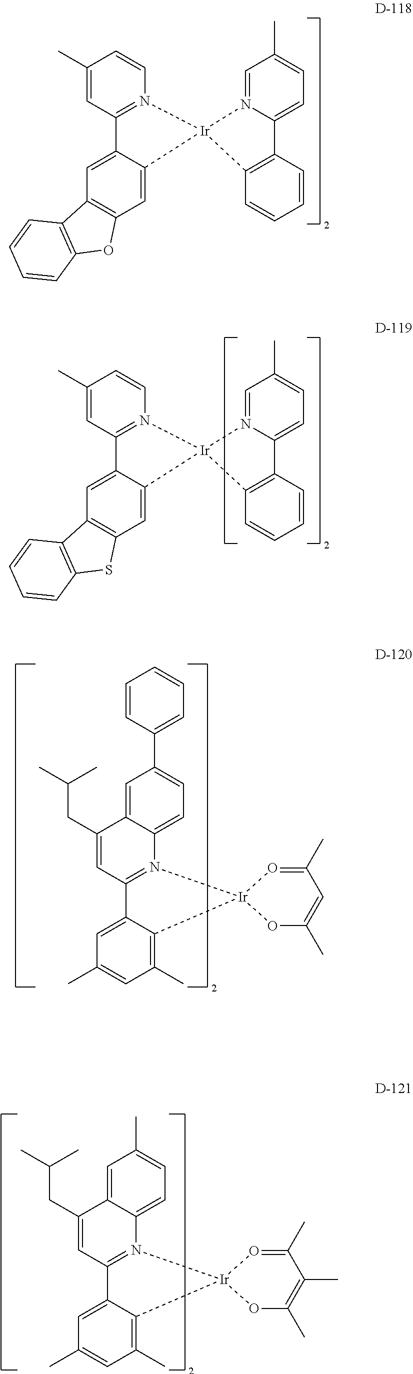

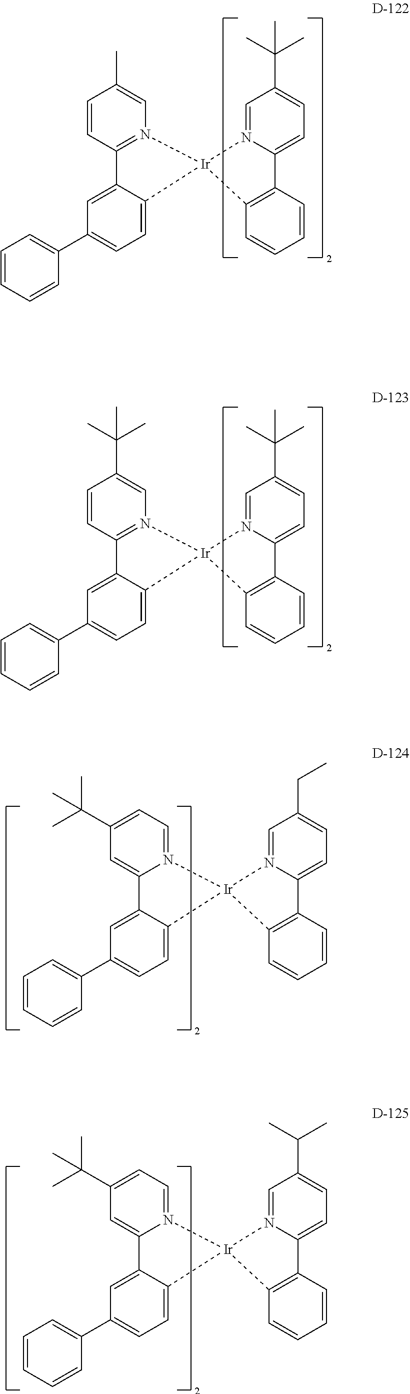

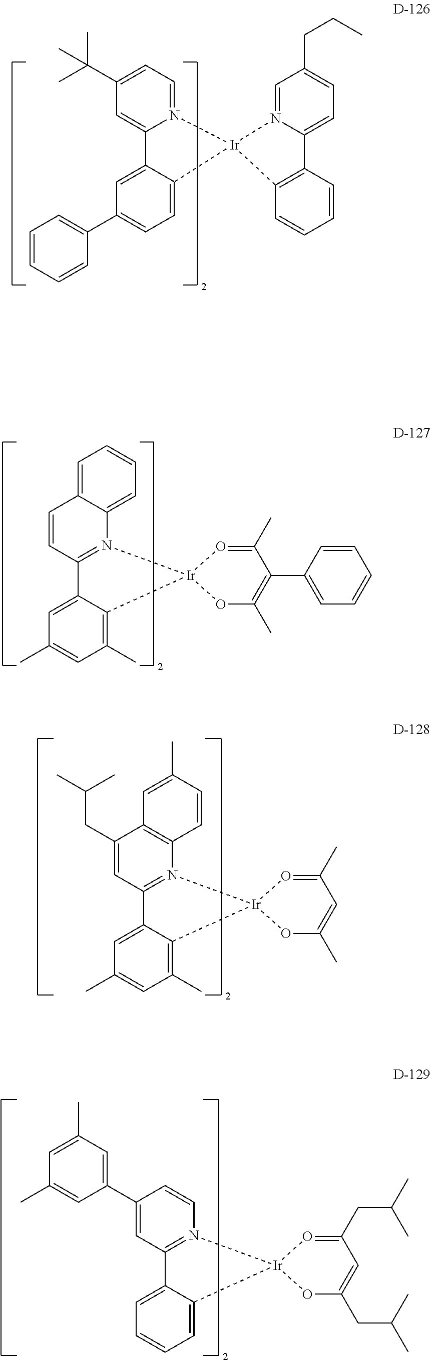

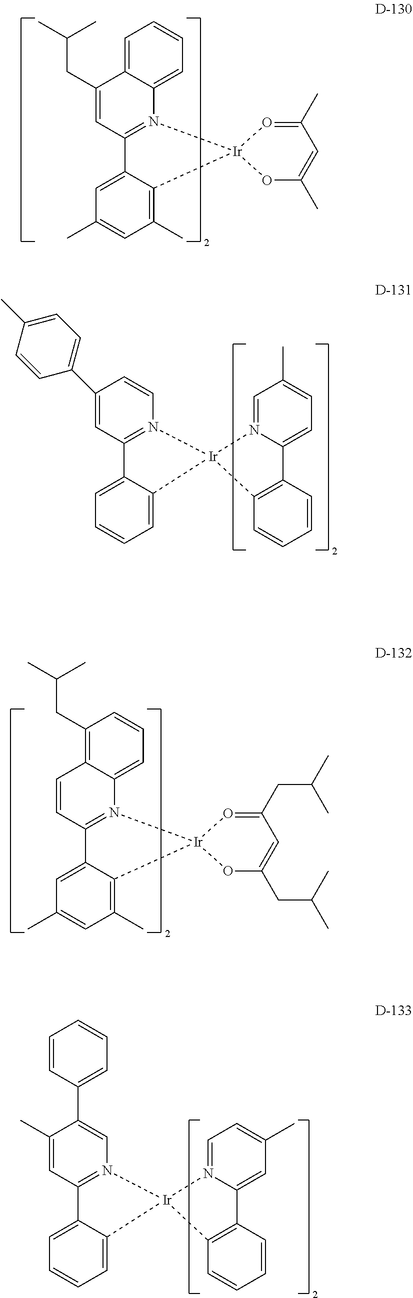

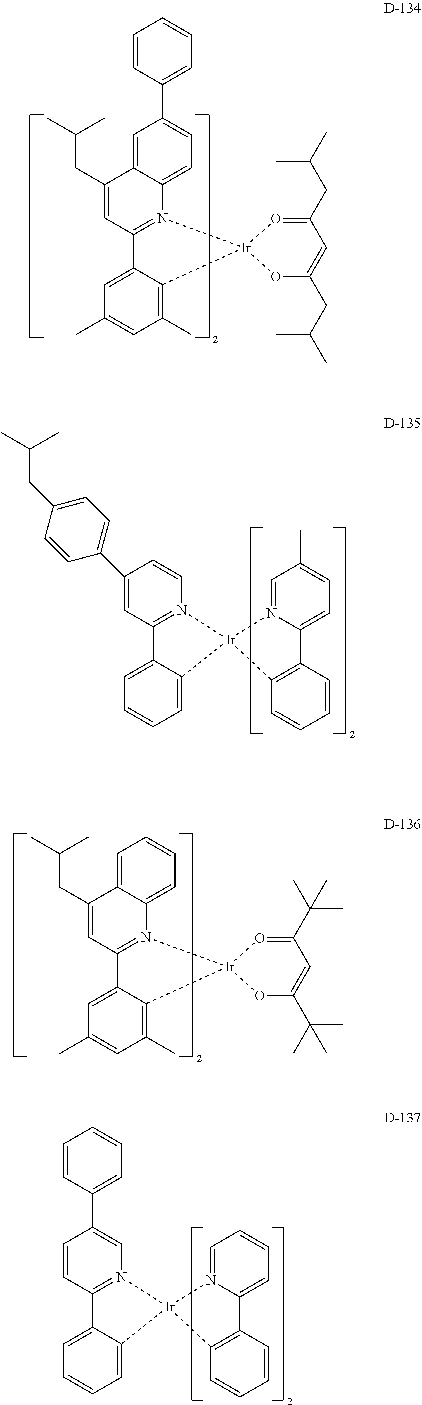

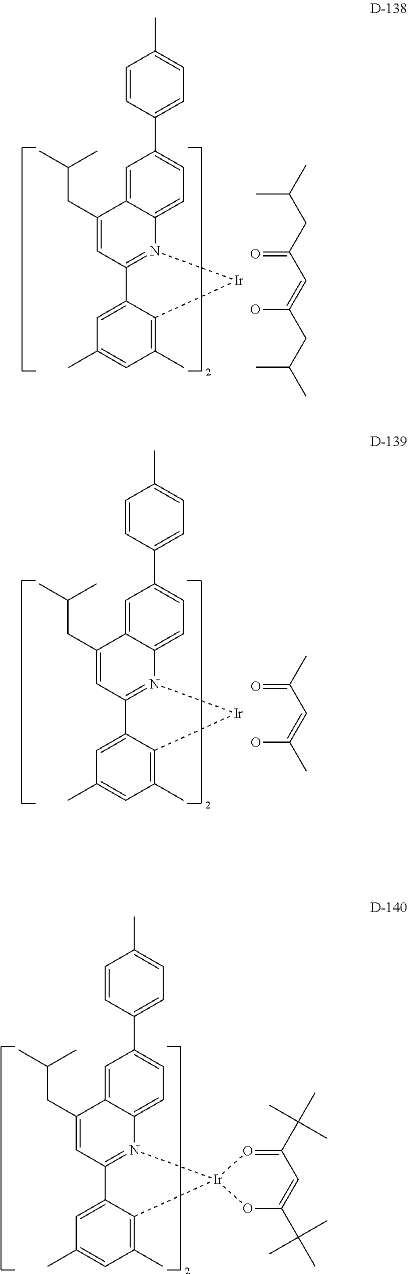

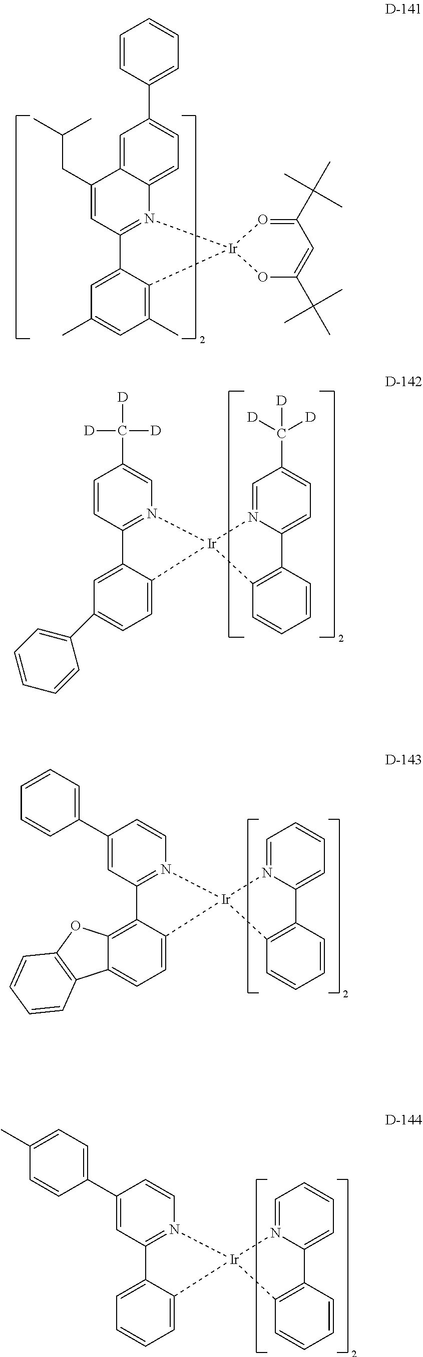

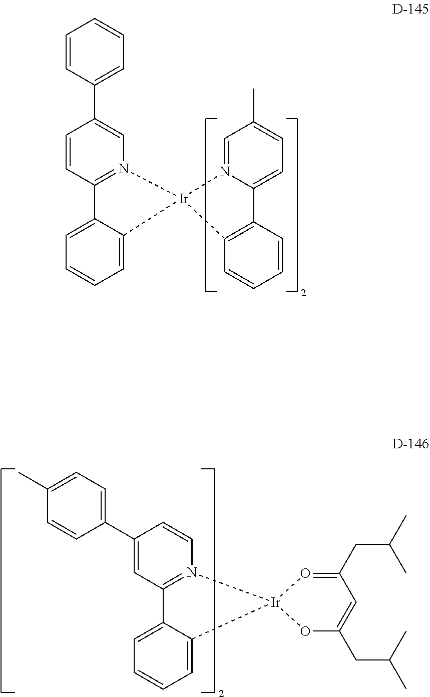

According to another aspect of the present disclosure, a material for preparing an organic electroluminescent device and an organic electroluminescent device comprising the material are provided. The material comprises the compound of formula 1. The material may be specifically for preparing a light-emitting layer of the organic electroluminescent device, and preferably for a host of a light-emitting layer of the organic electroluminescent device. When the compound of formula 1 of the present disclosure is comprised in the material, the material may further comprise the compound of formula 7. The material may be a composition or mixture. The material may further comprise a conventional material which has been comprised for an organic electroluminescent material.

According to another aspect of the present disclosure, a combination comprising the compound of formula 1 and the compound of formula 7 is provided. In the combination comprising the compound of formula 1 and the compound of formula 7, the weight ratio between them in the range of 1:99 to 99:1, preferabley 30:70 to 70:30 is advantageous in terms of driving voltage, lifespan, and luminous efficiency. The combination may further comprise at least one dopant. The dopant may be preferably a phosphorescent dopant, and specifically, may be selected from the compounds of formulae 101 to 103.

According to another embodiment, the present disclosure provides an organic electroluminescent device which comprises a first electrode, a second electrode, and one or more light-emitting layers disposed between the first and second electrodes; at least one of the one or more light-emitting layers comprises one or more dopant compounds and two or more host compounds; a first host compound of the host compounds is represented by formula 1; and a second host compound is represented by formula 7. Specifically, the dopant may be selected from the compounds of formulae 101 to 103.

The organic electroluminescent device of the present disclosure may further comprise at least one compound selected from the group consisting of arylamine-based compounds and styrylarylamine-based compounds in the organic layer.

In the organic electroluminescent device of the present disclosure, the organic layer may further comprise, in addition to the compound of formula 1, at least one metal selected from the group consisting of metals of Group 1, metals of Group 2, transition metals of the 4^{th} period, transition metals of the 5^{th} period, lanthanides and organic metals of the d-transition elements of the Periodic Table, or at least one complex compound comprising the metal. The organic layer may further comprise a light-emitting layer and a charge generating layer.

In the organic electroluminescent device of the present disclosure, preferably, at least one layer (hereinafter, “a surface layer”) may be placed on an inner surface(s) of one or both electrode(s), selected from a chalcogenide layer, a metal halide layer and a metal oxide layer. Specifically, a chalcogenide (includes oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiO_{X}(1≤X≤2), AlO_{X}(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

In the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an electroluminescent device having two or more light-emitting layers and emitting white light.

In the organic electroluminescent device of the present disclosure, a hole injection layer, a hole transport layer, an electron blocking layer, or a combination thereof may be disposed between the anode and the light-emitting layer, and the hole auxiliary layer or the auxiliary light-emitting layer may be disposed between the hole transport layer and the light-emitting layer. The hole injection layer may be composed of two or more layers in order to lower an energy barrier for injecting holes from the anode to a hole transport layer or an electron blocking layer (or a voltage for injecting a hole). Each of the layers may comprise two or more compounds. The hole transport layer or electron blocking layer may be composed of two or more layers.

An electron buffering layer, a hole blocking layer, an electron transport layer, an electron injection layer, or a combination thereof may be disposed between the light-emitting layer and the cathode. The electron buffering layer may be composed of two or more layers in order to control the electron injection and improve characteristics of interface between the light-emitting layer and the electron injection layer. Each of the layers may comprise two or more compounds. The hole blocking layer or electron transport layer may be composed of two or more layers, and each of the layers may comprise two or more compounds.

In order to form each layer of the organic electroluminescent device of the present disclosure, any of dry film-forming methods such as vacuum evaporation, sputtering, plasma and ion plating methods, or wet film-forming methods such as ink jet printing, nozzle printing, slot coating, spin coating, dip coating, and flow coating methods can be used. A co-evaporation or a mixture-evaporation is used for forming a film of the first host material and a film of the second host material.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

The co-evaporation indicates a process for two or more materials to be deposited as a mixture, by introducing each of the two or more materials into respective crucible cells, and applying electric current to the cells for each of the materials to be evaporated. Herein, a mixture-evaporation indicates a process for two or more materials to be deposited as a mixture, by mixing the two or more materials in one crucible cell before the deposition, and applying electric current to the cell for the mixture to be evaporated.

A display system or a lighting system using the organic electroluminescent device of the present disclosure can be produced.

Hereinafter, the organic electroluminescent compound of the present disclosure, the preparation method and the physical properties of the compound, and the luminescent properties of the organic electroluminescent device comprising the compound will be explained in detail with reference to the following examples.

### EXAMPLE 1: PREPARATION OF COMPOUND H1-2

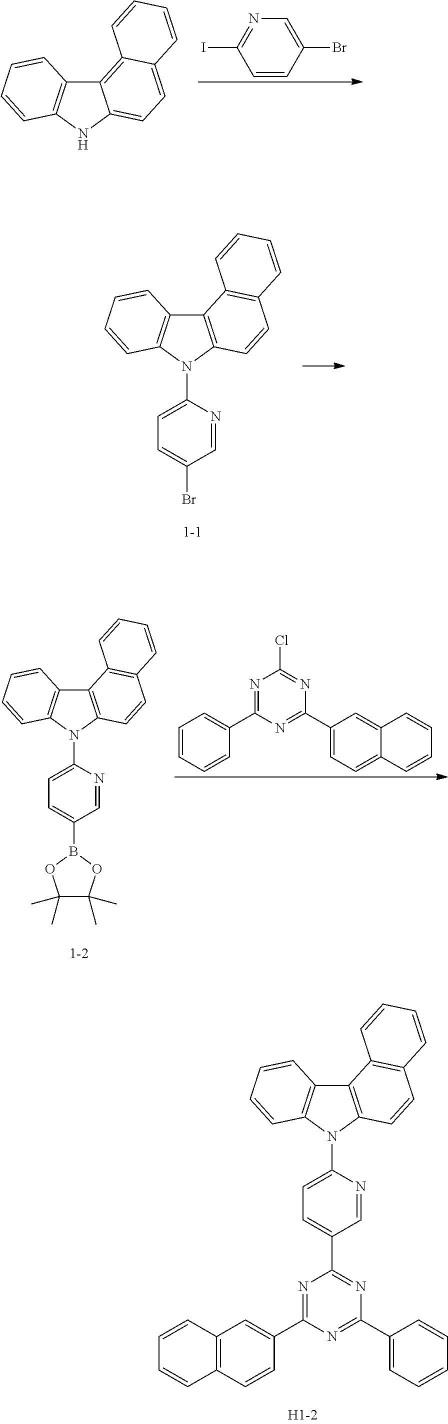

### 1) Preparation of Compound 1-1

After introducing a compound 7H-benzo[c]carbazole (30 g, 138.1 mmol), 5-bromo-2-iodopyridine (58.8 g, 207.1 mmol), CuI (12.5 g, 65.4 mmol), K₃PO₄ (73 g, 345.2 mmol), ethylene diamine (8.3 g, 138.1 mmol) and toluene (600 mL) into a flask, the mixture was stirred under reflux at 120° C. for 4 hours. After completion of the reaction, the mixture was extracted with ethyl acetate and purified water, and the obtained organic layer was concentrated under reduced pressure. The organic layer was subjected to silica gel column chromatography (methylene chloride(MC):hexane(Hex)) to obtain compound 1-1 (16 g, yield: 31%).

### 2) Preparation of Compound 1-2

After introducing compound 1-1 (16 g, 42.86 mmol), pinacolatodiboron (13.1 g, 51.44 mmol), PdCl₂ (PPh₃)₂ (3 g, 4.3 mmol), potassium acetate (KOAc) (10.5 g, 107 mmol) and 1,4-dioxane (200 mL) into a flask, the mixture was stirred under reflux at 120° C. for 2 hours. After completion of the reaction, the mixture was extracted with ethyl acetate and purified water, and the obtained organic layer was dried under reduced pressure. The organic layer was subjected to silica gel column chromatography (MC:Hex) to obtain compound 1-2 (11 g, yield: 61%).

### 3) Preparation of compound H1-2

After introducing compound 1-2 (11 g, 26.17 mmol), 2-chloro-4-(naphthalen-2-yl)-6-phenyl-1,3,5-triazine (8.3 g, 26.17 mmol), Na₂CO₃ (6.9 g, 65.42 mmol), Pd(PPh₃)₄ (1.5 g, 1.3 mmol), tetrahydrofuran (THF) (100 mL) and purified water (30 mL) into a flask, the mixture was stirred under reflux at 120° C. for 4 hours. After completion of the reaction, the mixture was extracted with ethyl acetate and purified water, and the obtained organic layer was concentrated under reduced pressure. The organic layer was subjected to silica gel column chromatography (MC:Hex) to obtain compound H1-2 (6.54 g, yield: 43.1%).

### EXAMPLE 2: PREPARATION OF COMPOUND H1-60

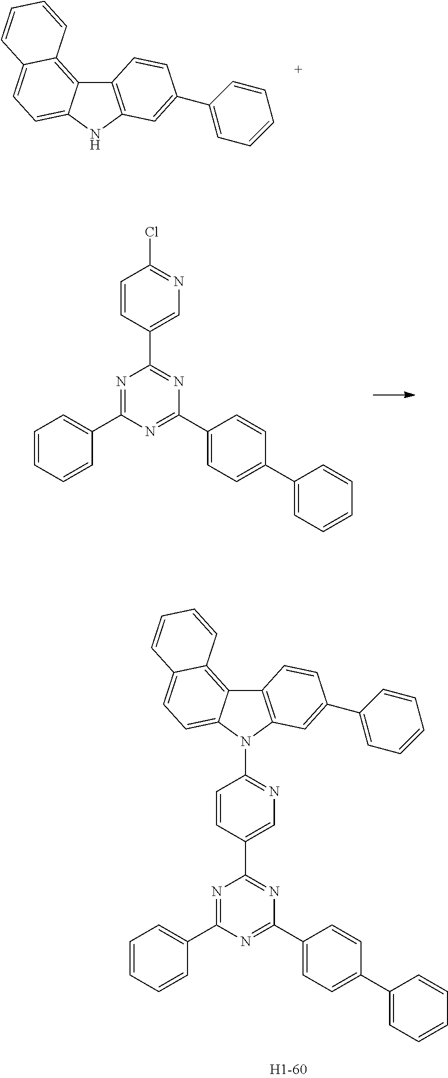 [SMILES: c1ccc(-c2ccc3c(c2)[nH]c2ccc4ccccc4c23)cc1 ; Clc1ccc(-c2nc(-c3ccccc3)nc(-c3ccc(-c4ccccc4)cc3)n2)cn1 ; c1ccc(-c2ccc(-c3nc(-c4ccccc4)nc(-c4ccc(-n5c6cc(-c7ccccc7)ccc6c6c7ccccc7ccc65)nc4)n3)cc2)cc1]

### 1) Preparation of Compound H1-60

2-Phenyl-9H-carbazole (1.0 g, 3.4 mmol), 2-([1,1′-biphenyl]-4-yl)-4-(6-chloropyridin-3-yl)-6-phenyl-1,3,5-triazine (1.6g, 3.7 mmol), palladium(II) acteate (Pd(OAc)₂) (39 mg, 0.17 mmol), SPhos (0.14 g, 0.34 mmol), sodium tert-butoxide (NaOtBu) (0.816 g, 8.5 mmol) and o-xylene (17 mL) were added dropwise to a flask, and then stirred under reflux at 175° C. for 4 hours. After completion of the reaction, the mixture was extracted with MC, and then dried with MgSO₄. After separation with column chromatography, MeOH was added to the resultant to obtain a solid, and the obtained solid was filtered under reduced pressure to obtain compound H1-60 (1.0 g, yield: 43%).

¹H NMR (600 MHz, CDCl₃, δ) 10.133-10.129(d,1H), 9.291-9.288(dd,1H), 8.891-8.874(dd,3H), 8.837-8.823(d,2H), 8.707-8.693(d,1H), 8.285-8.283(d,1H), 8.162-8.147(d,1H), 8.052-8.039(d,1H), 7.949-7.926(dd,2H), 7.846-7.832(d,2H), 7.756-7.721(m,6H), 7.663-7.622(m,3H), 7.545-7.472(m,5H), 7.437-7.415(t,1H), 7.381-7.369(t,1H)

| | MW | | | M.P |
|---|---|---|---|---|
| | (Molecular Weight) | UV | PL | (Melting Point) |
| H1-60 | 677.81 | 394 nm | 532 nm | 229° C. |

### EXAMPLE 3: PREPARATION OF COMPOUND H1-68

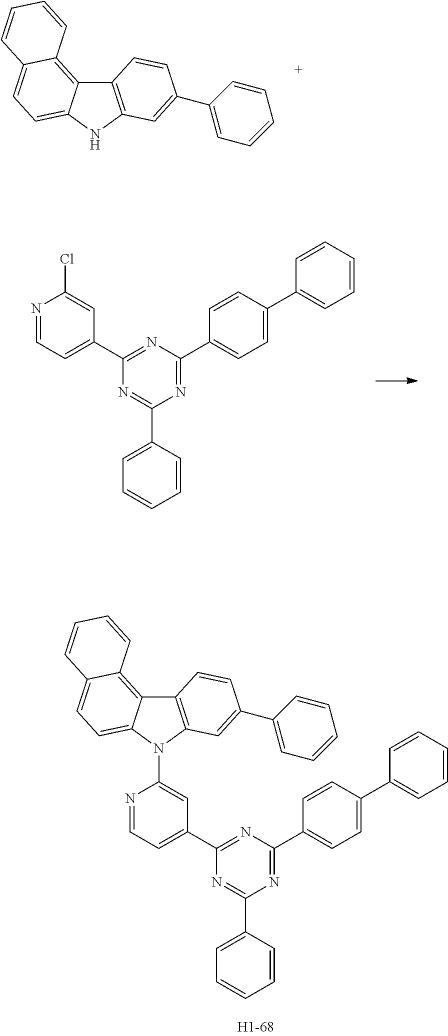 [SMILES: c1ccc(-c2ccc3c(c2)[nH]c2ccc4ccccc4c23)cc1 ; Clc1cc(-c2nc(-c3ccccc3)nc(-c3ccc(-c4ccccc4)cc3)n2)ccn1 ; c1ccc(-c2ccc(-c3nc(-c4ccccc4)nc(-c4ccnc(-n5c6cc(-c7ccccc7)ccc6c6c7ccccc7ccc65)c4)n3)cc2)cc1]

### 1) Preparation of Compound H1-68

2-Phenyl-9H-carbazole (4.0 g, 13.6 mmol), 2-([1,1′-biphenyl]-4-yl)-4-(2-chloropyridin-4-yl)-6-phenyl-1,3,5-triazine (6.3 g, 15 mmol), palladium(II) acteate (Pd(OAc)₂) (153 mg, 0.68 mmol), SPhos (0.558 g, 1.36 mmol), sodium tert-butoxide (NaOtBu) (3.3 g, 34 mmol) and o-xylene (70 mL) were added dropwise to a flask, and then stirred under reflux at 180° C. for 4 hours. After completion of the reaction, the mixture was extracted with MC, and then dried with MgSO₄. After separation with column chromatography, MeOH was added to the resultant to obtain a solid, and the obtained solid was filtered under reduced pressure to obtain compound H1-68 (2.2 g, yield: 23.9%).

¹H NMR (600 MHz, CDCl₃, δ) 9.061-9.054(m,2H), 8.926-8.912(d,1H), 8.833-8.819(d,2H), 8.789-8.777(d,2H), 8.750-8.736(d,1H), 8.662-8.652(d,1H), 8.265-8.263(d,1H), 8.157-8.142 (d,1H), 7.950-7.936(d,1H), 7.789-7.767(m,6H), 7.701-7.689(d,2H), 7.652-7.628(t,1H), 7.579-7.540(m,3H), 7.515-7.490(t,2H), 7.434-7.390(m,3H), 7.327-7.303(t,2H)

| | MW | UV | PL | M.P |
|---|---|---|---|---|
| H1-68 | 677.81 | 384 nm | 472 nm | 284.5° C. |

### EXAMPLE 4: PREPARATION OF COMPOUND H1-69

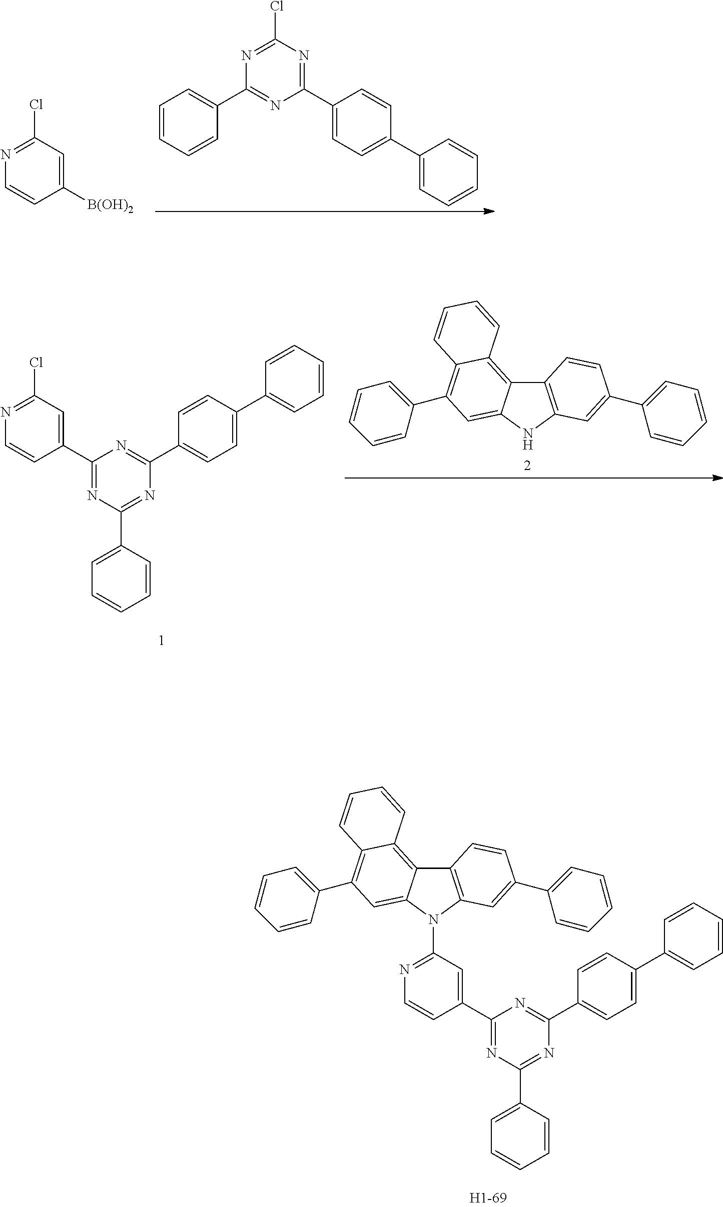

### 1) Preparation of Compound 1

After dissolving (2-chloropyridin-4-yl)boronic acid (10.0 g, 63.5 mmol), 2-([1,1′-biphenyl]-4-yl)-4-chloro-6-phenyl-1,3,5-triazine (32.8 g, 95.3 mmol), Pd(PPh₃)₄ (3.7 g, 3.2 mmol), and K₂CO₃ (17.6 g, 127 mmol) in toluene (320 mL), EtOH (80 mL), and H₂O (80 mL) of a flask, the mixture was under reflux at 130° C. for 5 hours. After completion of the reaction, the mixture was extracted with ethyl acetate, and then the obtained organic layer was dried with MgSO₄ to remove the remaining moisture, and subjected to column chromatography to obtain compound 1 (13.2 g, yield: 50%).

### 2) Preparation of Compound H1-69

After introducing compound 1 {2-([1,1′-biphenyl]-4-yl)-4-(2-chloropyridin-4-yl)-6-phenyl-1,3,5-triazine} (5.2 g, 12.4 mmol), compound 2 {5,9-diphenyl-7H-benzo[c]carbazole} (4.2 g, 11.3 mmol), palladium(II) acteate (Pd(OAc)₂) (0.13 g, 0.56 mmol), SPhos (0.46 g, 1.13 mmol), sodium tert-butoxide (NaOtBu) (2.7 g, 28.3 mmol), and o-xylene (87 mL) into a flask, said compounds were dissolved and the mixture was under reflux at 150° C. for 12 hours. After completion of the reaction, the mixture was extracted with ethyl acetate, and then the obtained organic layer was dried with MgSO₄ to remove the remaining moisture, and subjected to column chromatography to obtain compound H1-69 (5.2 g, yield: 61%).

¹H NMR (600 MHz, CDCl₃, δ) 9.07(s,1H), 9.015-9.004(d,2H), 8.826-8.808(m,2H), 8.785-8.771(d,3H), 8.613-8.599(m,1H), 8.310(s,1H), 8.118(s,1H), 8.076-8.062(d,1H), 7.806-7.768(m,6H), 7.710-7.698(d,2H), 7.663-7.625(m,3H), 7.588-7.563(t,2H), 7.530-7.492(m,3H), 7.474-7.406(m,6H), 7.351-7.326(m,1H)

| | MW | UV | PL | M.P |
|---|---|---|---|---|
| H1-69 | 753.91 | 362 nm | 413 nm | 170° C. |

### EXAMPLE 5: PREPARATION OF COMPOUND H1-47

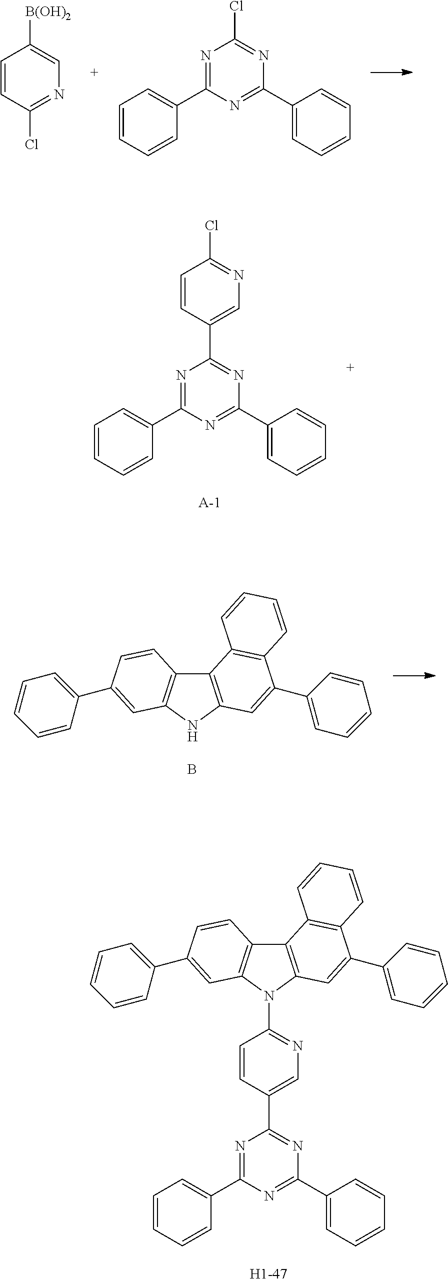

### 1) Preparation of Compound A-1

After dissolving (6-chloropyridin-3-yl)boronic acid (5 g, 32 mmol), 2-chloro-4,6-diphenyl-1,3,5-triazine (12.76 g, 48 mmol), Pd(PPh₃)₄ (1.8 g, 2 mmol), and K₂CO₃ (13 g, 64 mmol) in toluene (100 mL), ethanol (31 mL), and water (31 mL) in a flask, the mixture was under reflux at 120° C. for 5 hours. The resultant solid was filtered, and the obtained solid was washed with methanol to obtain compound A-1 (9.8 g, yield: 89%).

### 2) Preparation of Compound H1-47

After dissolving compound A-1 (9 g, 24 mmol), compound B (8.8 g, 26 mmol), palladium(II) acteate (Pd(OAc)₂) (0.273 g, 1 mmol), SPhos (1 g, 2 mmol), and sodium tert-butoxide (NaOtBu) (5.83 g, 61 mmol) in xylene (240 mL) in a flask, the mixture was under reflux at 150° C. for 2 hours. After completion of the reaction, the mixture was extracted with ethyl acetate, and then the obtained organic layer was dried with MgSO₄ to remove the remaining moisture, and subjected to column chromatography to obtain compound H1-47 (10 g, yield: 60%).

¹H NMR (600 MHz, CDCl₃, δ) 10.079-10.075 (sd, J=2.4 Hz, 1H), 9.254-9.236 (dd, J=8.4 Hz, 1H), 8.973-8.959 (d, J=8.4 Hz, 1H), 8.807-8.793 (m, 4H), 8.738-8.724 (d, J=8.4 Hz, 1H), 8.284 (s, 1H), 8.094 (s, 1H), 8.045-8.032 (d, J=7.8 Hz, 1H), 7.950-7.936 (d, J=8.4 Hz, 1H), 7.778-7.753 (m, 4H), 7.644-7.597 (m, 8H), 7.529-7.465 (m, 6H), 7.453-7.372 (m, 1H)

| | MW | UV | PL | M.P |
|---|---|---|---|---|
| H1-47 | 677.81 | 410 nm | 477 nm | 333° C. |

### [Device Example 1-1] OLED Produced by Evaporation of the Compound of the Present Disclosure as a Host

An OLED was produced using the organic electroluminescent compound of the present disclosure as follows. A transparent electrode indium tin oxide (ITO) thin film (10 Ω/sq) on a glass substrate for an OLED (Geomatec) was subjected to an ultrasonic washing with acetone, ethanol, and distilled water sequentially, and was then stored in isopropanol. The ITO substrate was then mounted on a substrate holder of a vacuum vapor depositing apparatus. HIL-1 was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 80 nm on the ITO substrate. HIL-2 was then introduced into another cell of said vacuum vapor depositing apparatus, and evaporated by applying electric current to the cell, thereby forming a second hole injection layer having a thickness of 5 nm on the first hole injection layer. HTL-1 was introduced into one cell of the vacuum vapor depositing apparatus, and evaporated by applying electric current to the cell, thereby forming a first hole transport layer having a thickness of 10 nm on the second hole injection layer. HTL-2 was introduced into another cell of the vacuum vapor depositing apparatus, and evaporated by applying electric current to the cell, thereby forming a second hole transport layer having a thickness of 60 nm on the first hole transport layer. After forming the hole injection layer and the hole transport layer, a light-emitting layer was deposited thereon as follows. Compound H1-2 was introduced, as a host material, into a cell of the vacuum vapor depositing apparatus, and compound D-71 was introduced, as a dopant, into another cell. The two compounds were then evaporated at different rates, so that the dopant was deposited in a doping amount of 3 wt % based on the total amount of the host and dopant to form a light-emitting layer having a thickness of 40nm on the second hole transport layer. Compounds ETL-1 and Liq were then introduced into another two cells of the vacuum vapor depositing apparatus, respectively, and evaporated at the same rate of 1:1, thereby forming an electron transport layer having a thickness of 30nm on the light-emitting layer. After depositing compound Liq as an electron injection layer having a thickness of 2 nm on the electron transport layer, an Al cathode having a thickness of 80 nm was then deposited by another vacuum vapor deposition apparatus on the electron injection layer to produce an OLED.

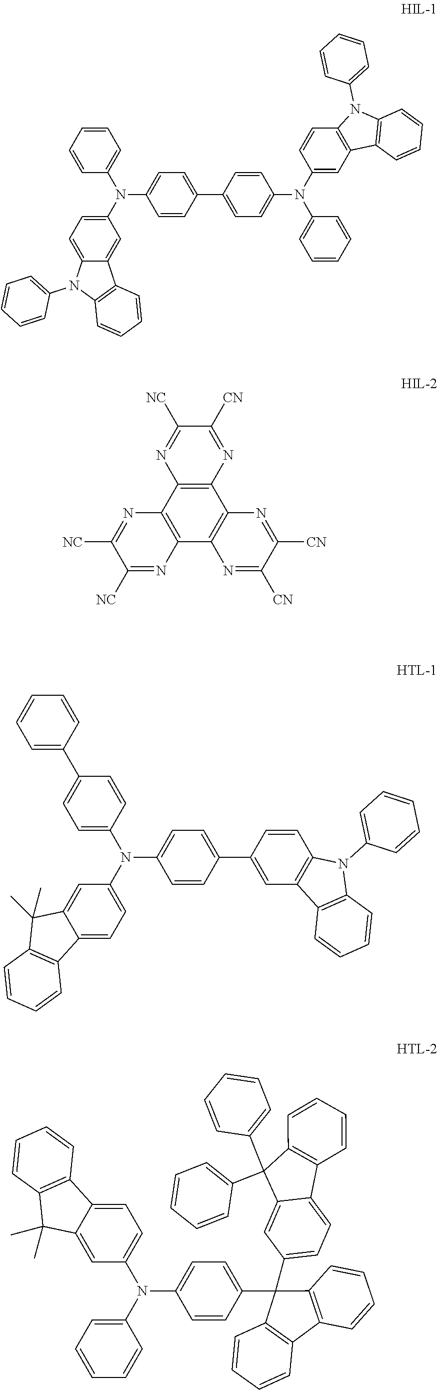 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(C4(c5ccc6c(c5)C(c5ccccc5)(c5ccccc5)c5ccccc5-6)c5ccccc5-c5ccccc54)cc3)cc21]

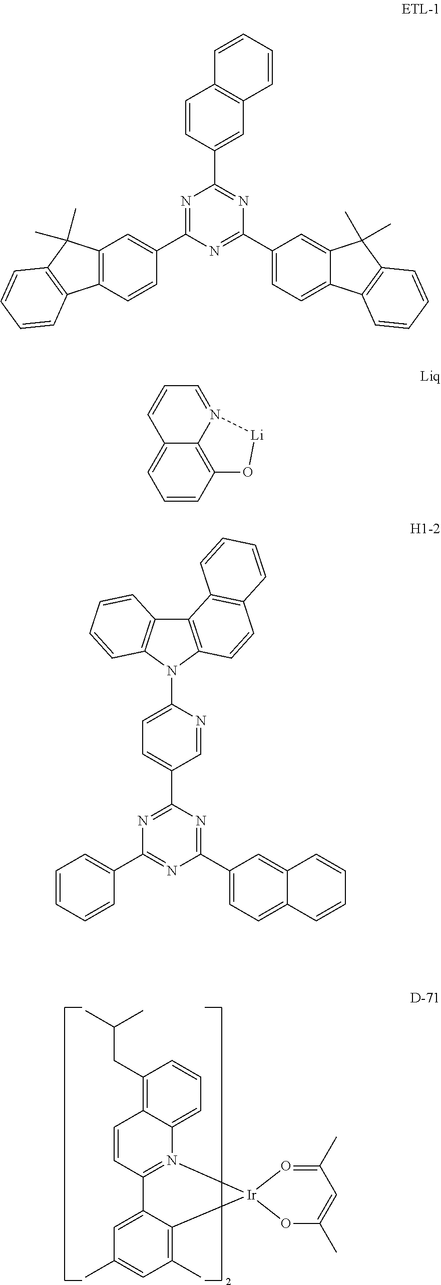

### [Device Examples 1-2 and 1-4] OLED Produced by Evaporation of the Compound of the Present Disclosure as a Host

An OLED was produced in the same manner as in Device Example 1-1, except that compound D-134 was used as a dopant for a light-emitting layer and the hosts of Device Examples 1-2 and 1-4 shown in Table 1 below were used as a host for a light-emitting layer, respectively.

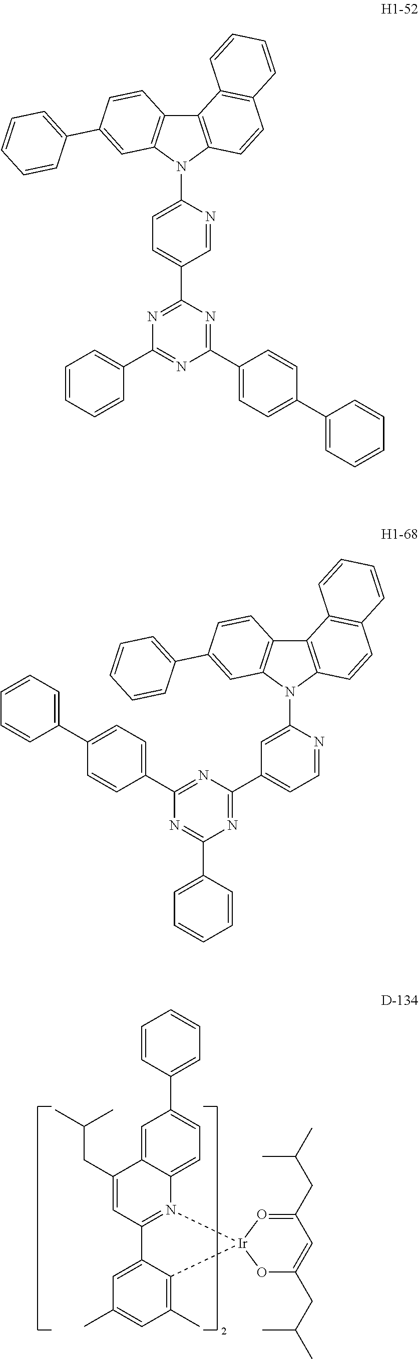

### [Device Examples 1-3 and 1-5] OLED Produced by Evaporation of the Compound of the Present Disclosure as a Host

An OLED was produced in the same manner as in Device Example 1-1, except that the hosts of Device Examples 1-3 and 1-5 shown in Table 1 below were used as a host for a light-emitting layer, respectively.

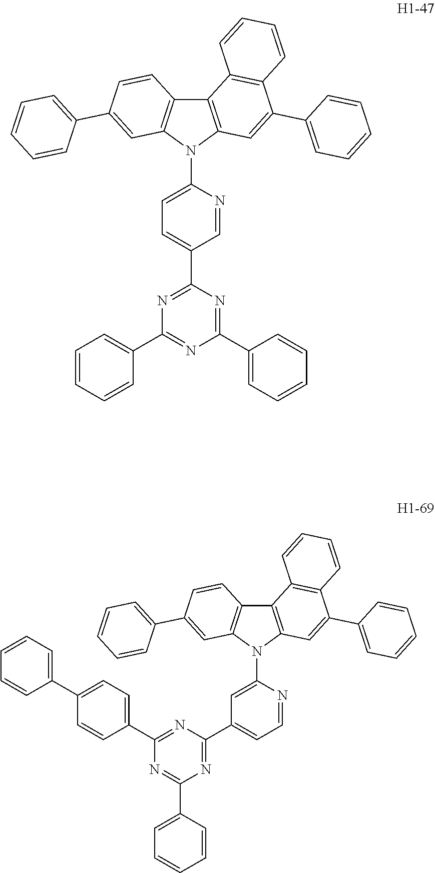 [SMILES: c1ccc(-c2ccc3c4c5ccccc5c(-c5ccccc5)cc4n(-c4ccc(-c5nc(-c6ccccc6)nc(-c6ccccc6)n5)cn4)c3c2)cc1 ; c1ccc(-c2ccc(-c3nc(-c4ccccc4)nc(-c4ccnc(-n5c6cc(-c7ccccc7)ccc6c6c7ccccc7c(-c7ccccc7)cc65)c4)n3)cc2)cc1]

### [Comparative Device Examples 1-1 and 1-2] OLED Using a Comparative Compound as a Host

An OLED was produced in the same manner as in Device Example 1-1, except that the hosts of Comparative Device Examples 1-1 and 1-2 shown in Table 1 below were used as a host for a light-emitting layer, respectively.

### [Comparative Device Examples 1-3 and 1-4] OLED Using a Comparative Compound as a Host

An OLED was produced in the same manner as in Device Example 1-2, except that the host of Device Examples 1-3 and 1-4 shown in Table 1 below were used as a host for a light-emitting layer.

The characteristics of the produced organic electroluminescent devices are shown in Table 1 below.

TABLE 1

| | | | The | T95 |
|---|---|---|---|---|
| | | | Emission | lifespan |
| Device Example No. | Host | Dopant | Color | [hr] |
| Device Example 1-1 | H1-2 | D-71 | Red | 45 |
| Device Example 1-2 | H1-52 | D-134 | Red | 51 |
| Device Example 1-3 | H1-47 | D-71 | Red | 30 |
| Device Example 1-4 | H1-68 | D-134 | Red | 58 |
| Device Example 1-5 | H1-69 | D-71 | Red | 64 |
| Comparative | Host-A | D-71 | Red | 19 |
| Device Example 1-1 | | | | |
| Comparative | Host-B | D-71 | Red | 20 |
| Device Example 1-2 | | | | |
| Comparative | Host-A | D-134 | Red | 17 |
| Device Example 1-3 | | | | |
| Comparative | Host-B | D-134 | Red | 19 |
| Device Example 1-4 | | | | |

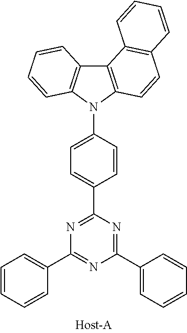 [SMILES: c1ccc(-c2nc(-c3ccccc3)nc(-c3ccc(-n4c5ccccc5c5c6ccccc6ccc54)cc3)n2)cc1]

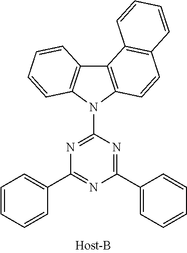 [SMILES: c1ccc(-c2nc(-c3ccccc3)nc(-n3c4ccccc4c4c5ccccc5ccc43)n2)cc1]

In Table 1 above, T95 lifespan indicates the time taken until an initial photocurrent under 500 nit luminance set at 100% is reduced to 95%.

Table 1 shows that the organic electroluminescent devices using the organic electroluminescent compound of the present disclosure as as a host for a light-emitting layer have a lifespan which is remarkably improved than that of the organic electroluminescent devices using the conventional organic electroluminescent compound.

### [Device Examples 1-6 to 1-9] OLED Using a Plurality of Host Materials Including the Compound of the Present Disclosure as a Host

An OLED was produced in the same manner as in Device Example 1-1, except that the first and second hosts shown in Table 2 below were introduced into each of two cells of the vacuum vapor depositing apparatus, respectively, and compound D-71 was introduced, as a dopant, into another cell, and then the two hosts were evaporated at the weight ratio of 1:1, so that the dopant was deposited in a doping amount of 3 wt % based on the total amount of the hosts and dopant to form a light-emitting layer having a thickness of 40nm on the second hole transport layer.

The characteristics of the produced organic electroluminescent devices under 1000 nit are shown in Table 2 below. In Table 2, T97 lifespan indicates the time taken until an initial photocurrent under 500 nit luminance set at 100% is reduced to 97%.

TABLE 2

| Device | | | Driving | Effi- | The | T97 |
|---|---|---|---|---|---|---|
| Example | The 1^{st} | The 2^{nd} | voltage | ciency | Emission | lifespan |
| No. | host | host | (V) | (cd/A) | Color | (hr) |
| Device | H2-6 | H1-2 | 3.4 | 28.5 | Red | 122 |
| Example | | | | | | |
| 1-6 | | | | | | |
| Device | H2-33 | H1-2 | 3.6 | 29.4 | Red | 161 |
| Example | | | | | | |
| 1-7 | | | | | | |
| Device | H2-8 | H1-2 | 3.7 | 30.1 | Red | 137 |
| Example | | | | | | |
| 1-8 | | | | | | |
| Device | H2-34 | H1-2 | 3.8 | 29.5 | Red | 129 |
| Example | | | | | | |
| 1-9 | | | | | | |

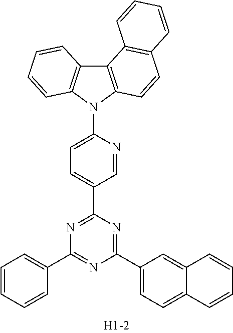 [SMILES: c1ccc(-c2nc(-c3ccc(-n4c5ccccc5c5c6ccccc6ccc54)nc3)nc(-c3ccc4ccccc4c3)n2)cc1]

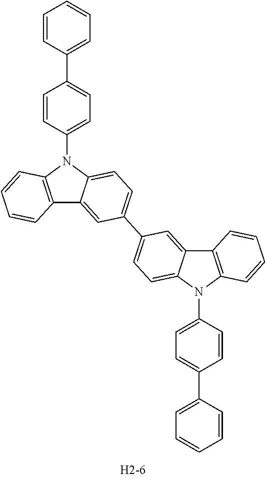 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

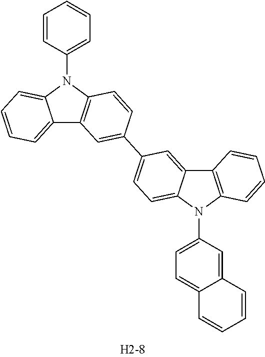 [SMILES: c1ccc(-n2c3ccccc3c3cc(-c4ccc5c(c4)c4ccccc4n5-c4ccc5ccccc5c4)ccc32)cc1]

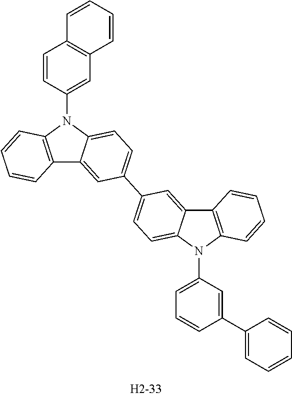 [SMILES: c1ccc(-c2cccc(-n3c4ccccc4c4cc(-c5ccc6c(c5)c5ccccc5n6-c5ccc6ccccc6c5)ccc43)c2)cc1]

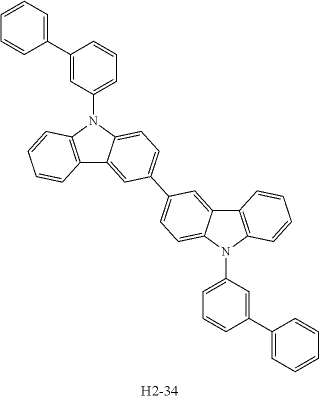 [SMILES: c1ccc(-c2cccc(-n3c4ccccc4c4cc(-c5ccc6c(c5)c5ccccc5n6-c5cccc(-c6ccccc6)c5)ccc43)c2)cc1]

### [Comparative Device Examples 1-5 and 1-6] OLED Using a Plurality of Host Materials but not Including the Compound of the Present Disclosure as the Host

An OLED was produced in the same manner as in Device Examples 1-6 to 1-9, except that the hosts shown in Table 3 below were used as a host for a light-emitting layer.

TABLE 3

| Device | The | The | Driving | | The | T97 |
|---|---|---|---|---|---|---|
| Example | 1^{st} | 2^{nd} | voltage | Efficiency | Emission | lifespan |
| No. | host | host | (V) | (cd/A) | Color | (hr) |
| Comparative | H2-6 | None | 8.6 | 2.8 | Red | X |
| Device | | | | | | |
| Example 1-5 | | | | | | |
| Comparative | H2-6 | Host- | 3.4 | 29.3 | Red | 26 |
| Device | | A | | | | |
| Example 1-6 | | | | | | |

* X indicates that lifespan of a device cannot be measured since efficiency is too low.

Tables 2 and 3 show that the organic electroluminescent devices, which use a plurality of host materials including the organic electroluminescent compounds of the present disclosure, have good lifespan.

1. An organic electroluminescent device comprising a first electrode, a second electrode, and one or more light-emitting layers disposed between the first and second electrodes; whereas at least one of the one or more light-emitting layers comprises a first host compound represented by formula 1 and a second host compound represented by formula 7. 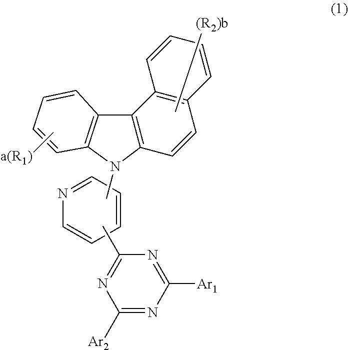
In formula 1,
Ar₁ and Ar₂, each independently, represent a substituted or unsubstituted (C6-C30)aryl, a substituted or unsubstituted (C3-C30)cycloalkyl, or a substituted or unsubstituted (3 to 30-membered)heteroaryl;
R₁ and R₂, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30) alkynyl, a substituted or unsubstituted (C3-C30)cycloalkyl, a substituted or unsubstituted (C6-C60)aryl, a substituted or unsubstituted (3 to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino;
a represents an integer of 1 to 4; b represents an integer of 1 to 6; when a or b is an integer of 2 or more, each of R₁ or each of R₂ are the same or different;
the heteroaryl contains at least one heteroatom selected from B, N, O, S, Si and P; 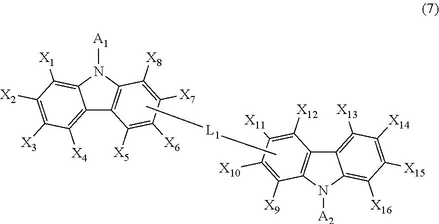
In formula 7,
A₁ represents a substituted or unsubstituted (C6-C30)aryl;
A₂ is selected from the group consisting of a substituted or unsubstituted biphenyl, a substituted or unsubstituted terphenyl, a substituted or unsubstituted naphthyl, a substituted or unsubstituted binaphthyl, a substituted or unsubstituted phenylnaphthyl, a substituted or unsubstituted naphthylphenyl, a substituted or unsubstituted fluorenyl, a substituted or unsubstituted phenylfluorenyl, a substituted or unsubstituted benzofluorenyl, a substituted or unsubstituted dibenzofluorenyl, a substituted or unsubstituted phenanthrenyl, a substituted or unsubstituted phenylphenanthrenyl, a substituted or unsubstituted anthracenyl, a substituted or unsubstituted indenyl, a substituted or unsubstituted triphenylenyl, a substituted or unsubstituted pyrenyl, a substituted or unsubstituted tetracenyl, a substituted or unsubstituted perylenyl, a substituted or unsubstituted chrysenyl, a substituted or unsubstituted naphthacenyl, a substituted or unsubstituted fluoranthenyl and a substituted or unsubstituted spirobifluorenyl;
L₁ represents a single bond or a substituted or unsubstituted (C6-C30)arylene; and
X₁ to X₁₆, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C60)aryl, a substituted or unsubstituted (3 to 30-membered)heteroaryl, a substituted or unsubstituted tri(C1-C30)alkylsilyl, a substituted or unsubstituted tri(C6-C30)arylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted mono- or di-(C1-C30)alkylamino, or a substituted or unsubstituted mono- or di-(C6-C30)arylamino; or may be linked to an adjacent substituent(s) to form a substituted or unsubstituted (C3-C30), mono- or polycyclic, alicyclic or aromatic ring, whose carbon atom(s) may be replaced with at least one heteroatom selected from nitrogen, oxygen, and sulfur.

2. The organic electroluminescent device according to claim 1, wherein the first host compound is represented by any one of the following formulae 2 to 6: 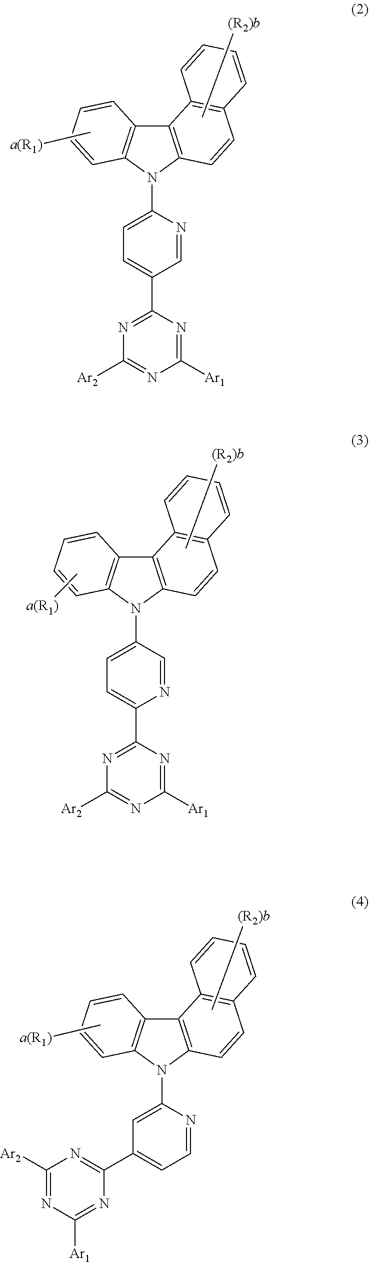 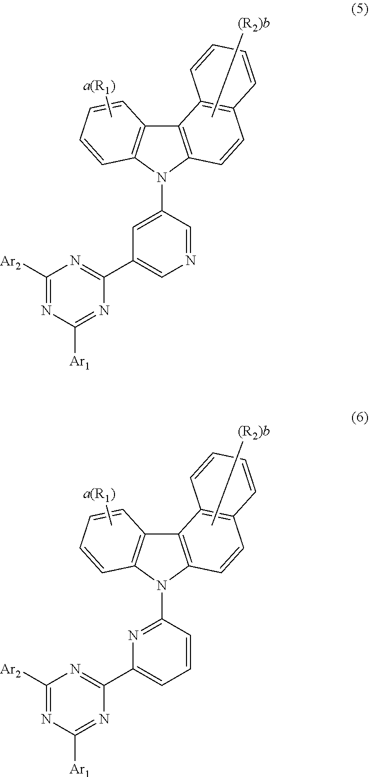
In formulae 2 to 6,
Ar₁, Ar₂, R₁, R₂, a and b are as defined in claim 1.

3. The organic electroluminescent deivce according to claim 1, wherein the substituent of the substituted (C6-C30)aryl, the substituted (C3-C30)cycloalkyl and the substituted (3 to 30-membered)heteroaryl in Ar₁ and Ar₂, and the substituent of the substituted (C1-C30)alkyl, the substituted (C2-C30)alkenyl, the substituted (C2-C30)alkynyl, the substituted (C3-C30)cycloalkyl, the substituted (C6-C60)aryl, the substituted (3 to 30-membered)heteroaryl, the substituted tri(C1-C30)alkylsilyl, the substituted tri(C6-C30)arylsilyl, the substituted di(C1-C30)alkyl(C6-C30)arylsilyl, the substituted (C1-C30)alkyldi(C6-C30)arylsilyl, the substituted mono- or di-(C1-C30)alkylamino, and the substituted mono- or di-(C6-C30)arylamino in R₁ and R₂, each independently, is at least one selected from the group consisting of deuterium, a halogen, a cyano, a carboxy, a nitro, a hydroxy, a (C1-C30)alkyl, a halo(C1-C30)alkyl, a (C2-C30)alkenyl, a (C2-C30) alkynyl, a (C1-C30)alkoxy, a (C1-C30)alkylthio, a (C3-C30)cycloalkyl, a (C3-C30)cycloalkenyl, a (3 to 7-membered)heterocycloalkyl, a (C6-C30)aryloxy, a (C6-C30)arylthio, a (5 to 30-membered)heteroaryl substituted or unsubstituted with a (C6-C30)aryl, a (C6-C30)aryl substituted or unsubstituted with a (5 to 30-membered)heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

4. The organic electroluminescent device according to claim 1, wherein Ar₁ and Ar₂ each independently, represent a substituted or unsubstituted phenyl, a substituted or unsubstituted naphthyl, a substituted or unsubstituted biphenyl, a substituted or unsubstituted terphenyl, a substituted or unsubstituted phenylnaphthyl, or a substituted or unsubstituted naphthylphenyl.

5. The organic electroluminescent device according to claim 1, wherein R₁ and R₂, each independently, represent hydrogen, a substituted or unsubstituted phenyl, a substituted or unsubstituted biphenyl, a substituted or unsubstituted naphthyl, a substituted or unsubstituted terphenyl, a substituted or unsubstituted phenylnaphthyl, or a substituted or unsubstituted naphthylphenyl.

6. The organic electroluminescent device according to claim 1, wherein the first host compound represented by formula 1 is selected from the group consisting of:

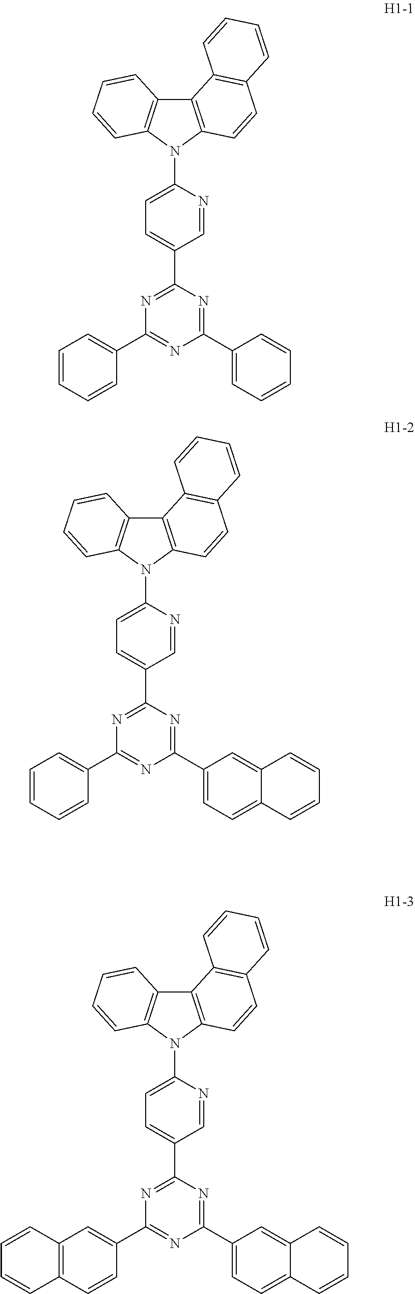 [SMILES: c1ccc(-c2nc(-c3ccccc3)nc(-c3ccc(-n4c5ccccc5c5c6ccccc6ccc54)nc3)n2)cc1 ; c1ccc(-c2nc(-c3ccc(-n4c5ccccc5c5c6ccccc6ccc54)nc3)nc(-c3ccc4ccccc4c3)n2)cc1 ; c1ccc2cc(-c3nc(-c4ccc(-n5c6ccccc6c6c7ccccc7ccc65)nc4)nc(-c4ccc5ccccc5c4)n3)ccc2c1]

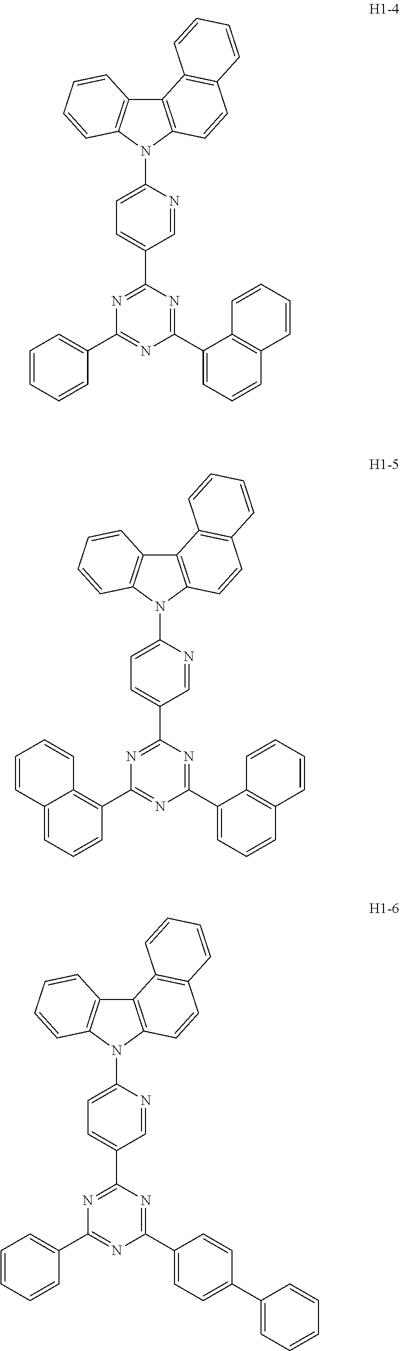 [SMILES: c1ccc(-c2nc(-c3ccc(-n4c5ccccc5c5c6ccccc6ccc54)nc3)nc(-c3cccc4ccccc34)n2)cc1 ; c1ccc2c(-c3nc(-c4ccc(-n5c6ccccc6c6c7ccccc7ccc65)nc4)nc(-c4cccc5ccccc45)n3)cccc2c1 ; c1ccc(-c2ccc(-c3nc(-c4ccccc4)nc(-c4ccc(-n5c6ccccc6c6c7ccccc7ccc65)nc4)n3)cc2)cc1]

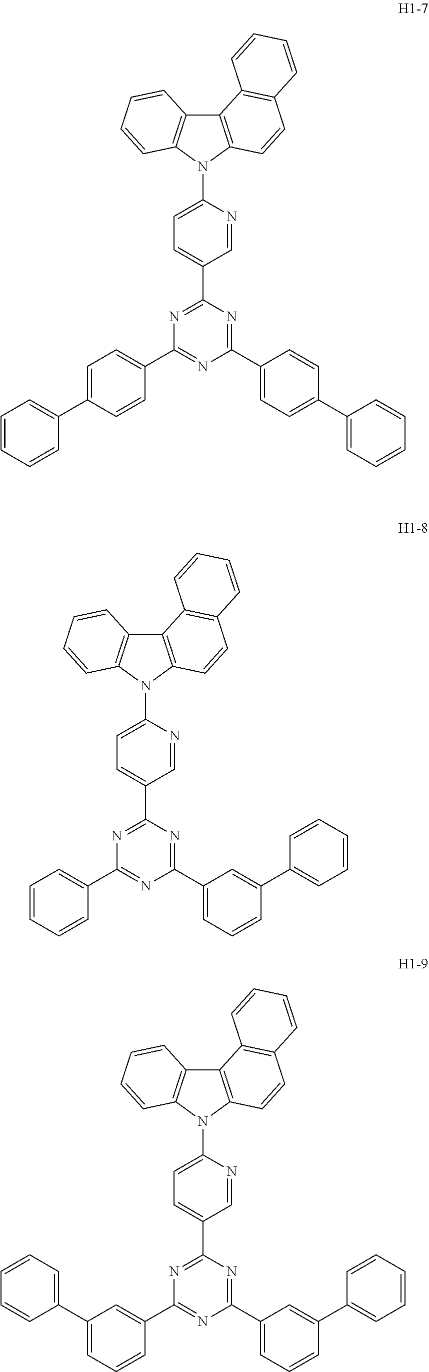 [SMILES: c1ccc(-c2ccc(-c3nc(-c4ccc(-c5ccccc5)cc4)nc(-c4ccc(-n5c6ccccc6c6c7ccccc7ccc65)nc4)n3)cc2)cc1 ; c1ccc(-c2cccc(-c3nc(-c4ccccc4)nc(-c4ccc(-n5c6ccccc6c6c7ccccc7ccc65)nc4)n3)c2)cc1 ; c1ccc(-c2cccc(-c3nc(-c4ccc(-n5c6ccccc6c6c7ccccc7ccc65)nc4)nc(-c4cccc(-c5ccccc5)c4)n3)c2)cc1]

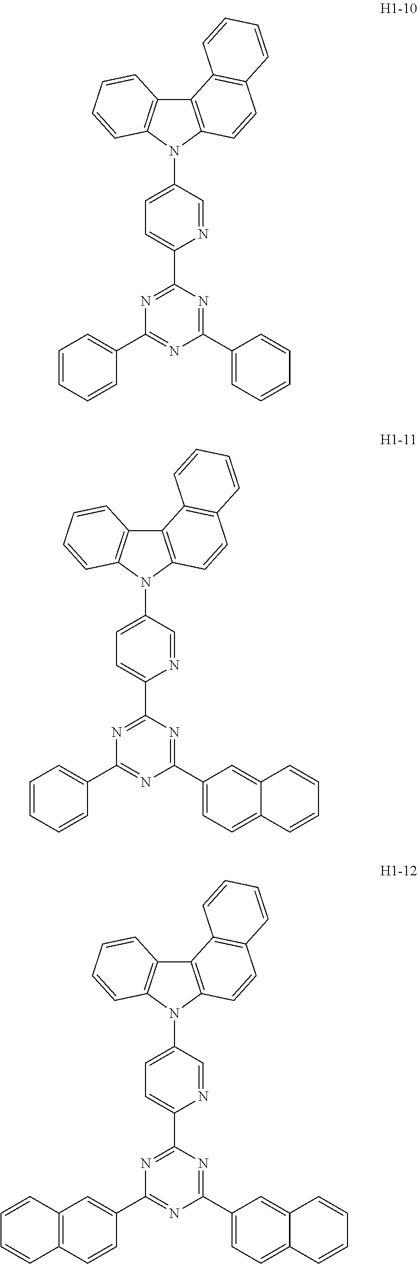 [SMILES: c1ccc(-c2nc(-c3ccccc3)nc(-c3ccc(-n4c5ccccc5c5c6ccccc6ccc54)cn3)n2)cc1 ; c1ccc(-c2nc(-c3ccc4ccccc4c3)nc(-c3ccc(-n4c5ccccc5c5c6ccccc6ccc54)cn3)n2)cc1 ; c1ccc2cc(-c3nc(-c4ccc5ccccc5c4)nc(-c4ccc(-n5c6ccccc6c6c7ccccc7ccc65)cn4)n3)ccc2c1]

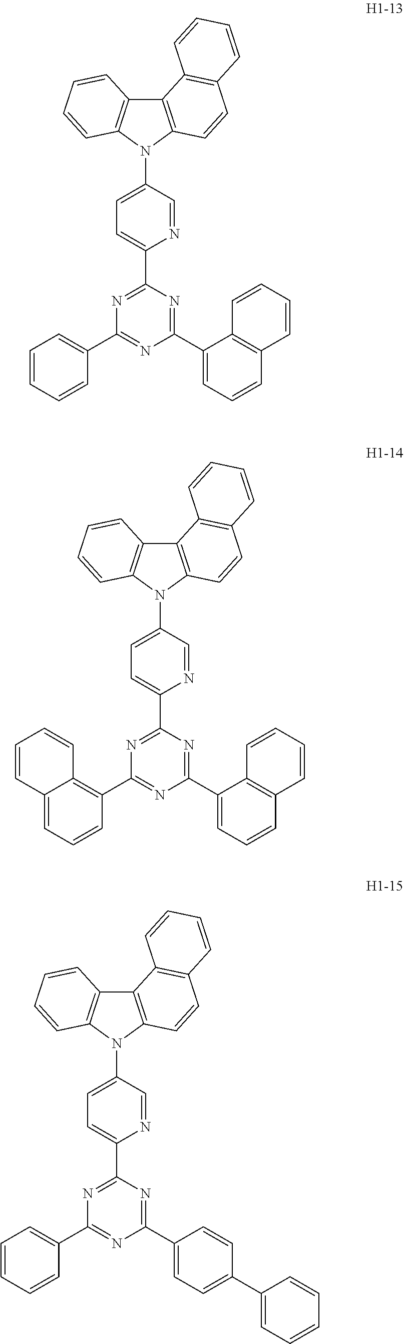 [SMILES: c1ccc(-c2nc(-c3ccc(-n4c5ccccc5c5c6ccccc6ccc54)cn3)nc(-c3cccc4ccccc34)n2)cc1 ; c1ccc2c(-c3nc(-c4ccc(-n5c6ccccc6c6c7ccccc7ccc65)cn4)nc(-c4cccc5ccccc45)n3)cccc2c1 ; c1ccc(-c2ccc(-c3nc(-c4ccccc4)nc(-c4ccc(-n5c6ccccc6c6c7ccccc7ccc65)cn4)n3)cc2)cc1]

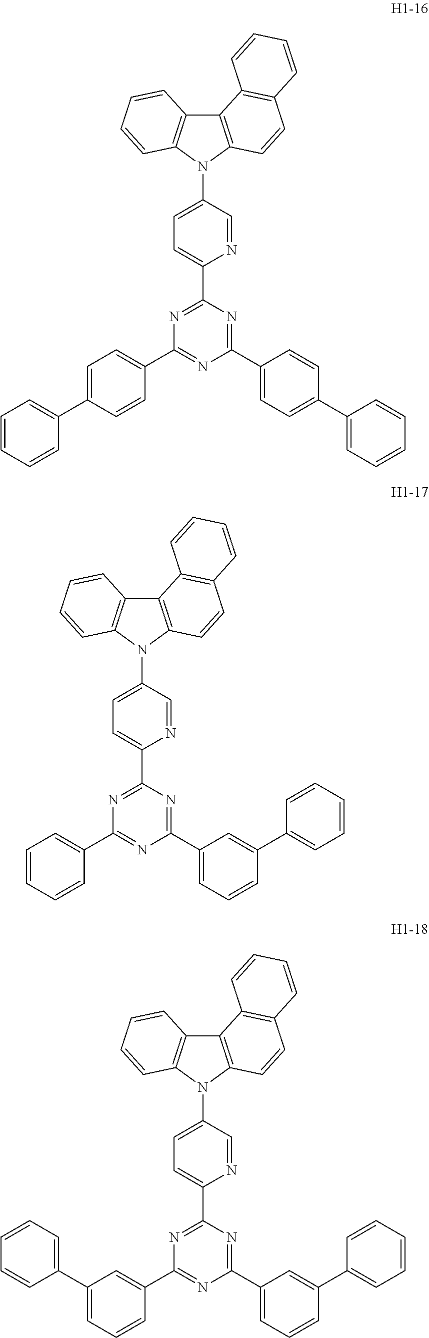 [SMILES: c1ccc(-c2ccc(-c3nc(-c4ccc(-c5ccccc5)cc4)nc(-c4ccc(-n5c6ccccc6c6c7ccccc7ccc65)cn4)n3)cc2)cc1 ; c1ccc(-c2cccc(-c3nc(-c4ccccc4)nc(-c4ccc(-n5c6ccccc6c6c7ccccc7ccc65)cn4)n3)c2)cc1 ; c1ccc(-c2cccc(-c3nc(-c4cccc(-c5ccccc5)c4)nc(-c4ccc(-n5c6ccccc6c6c7ccccc7ccc65)cn4)n3)c2)cc1]

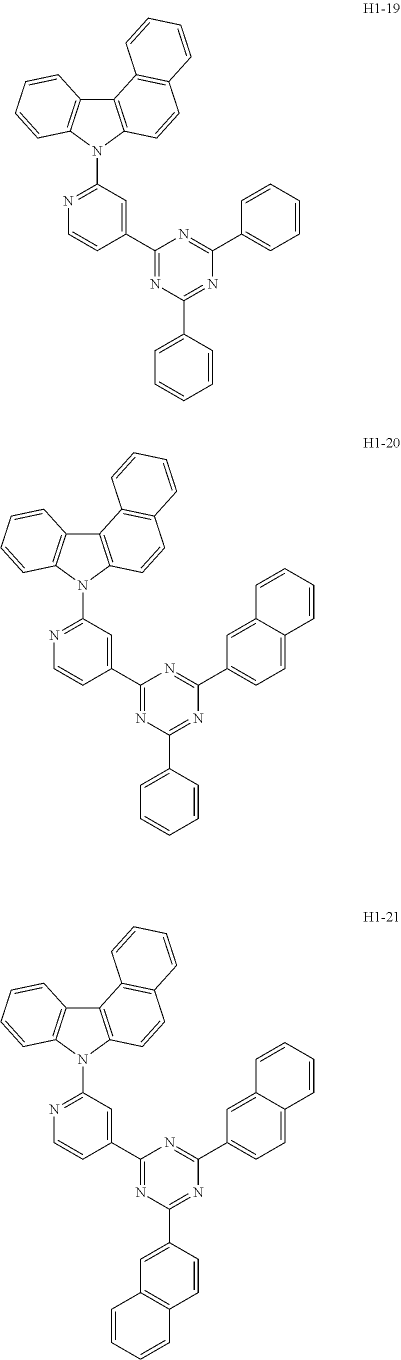 [SMILES: c1ccc(-c2nc(-c3ccccc3)nc(-c3ccnc(-n4c5ccccc5c5c6ccccc6ccc54)c3)n2)cc1 ; c1ccc(-c2nc(-c3ccnc(-n4c5ccccc5c5c6ccccc6ccc54)c3)nc(-c3ccc4ccccc4c3)n2)cc1 ; c1ccc2cc(-c3nc(-c4ccnc(-n5c6ccccc6c6c7ccccc7ccc65)c4)nc(-c4ccc5ccccc5c4)n3)ccc2c1]

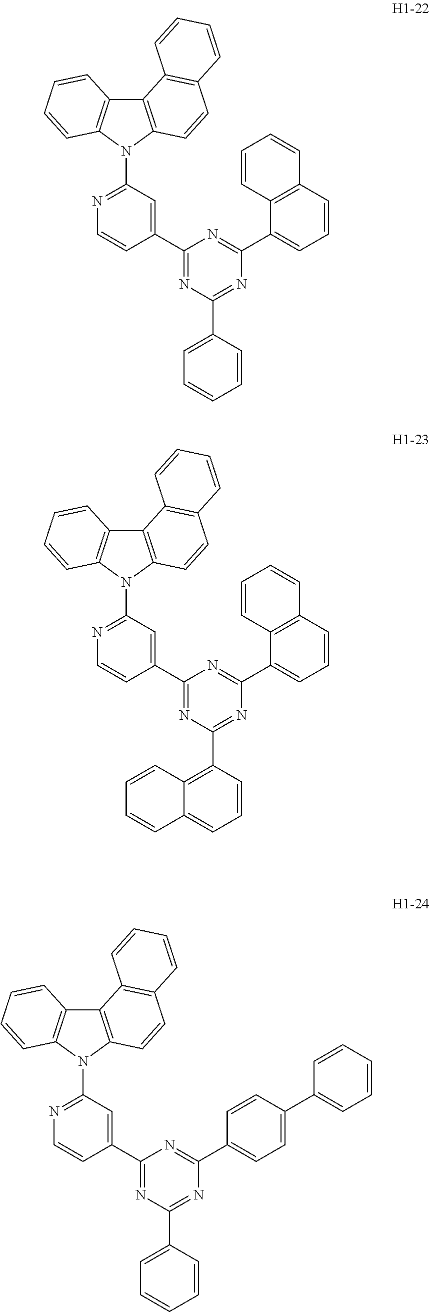 [SMILES: c1ccc(-c2nc(-c3ccnc(-n4c5ccccc5c5c6ccccc6ccc54)c3)nc(-c3cccc4ccccc34)n2)cc1 ; c1ccc2c(-c3nc(-c4ccnc(-n5c6ccccc6c6c7ccccc7ccc65)c4)nc(-c4cccc5ccccc45)n3)cccc2c1 ; c1ccc(-c2ccc(-c3nc(-c4ccccc4)nc(-c4ccnc(-n5c6ccccc6c6c7ccccc7ccc65)c4)n3)cc2)cc1]

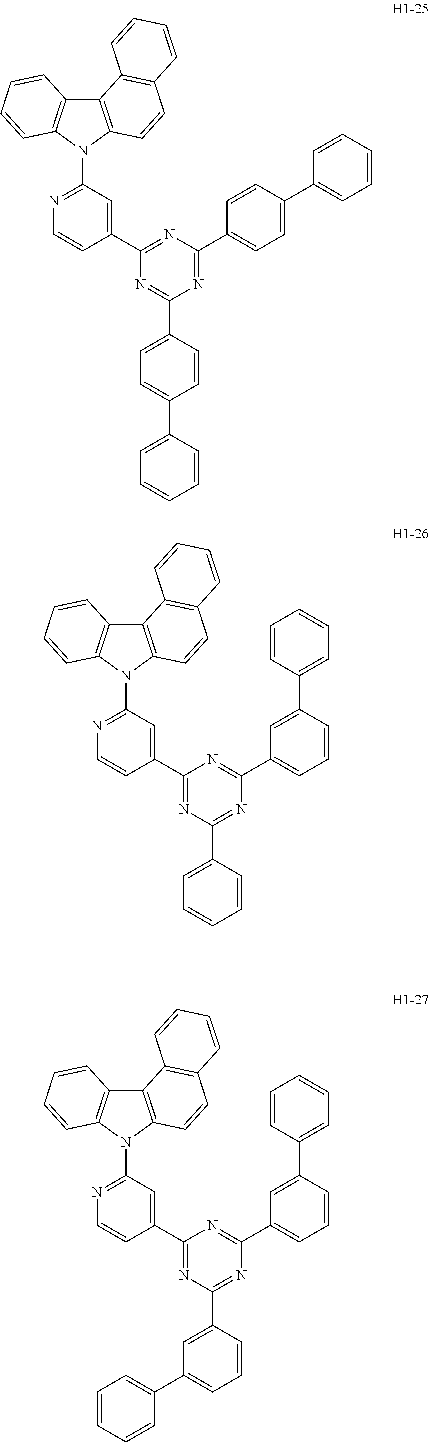

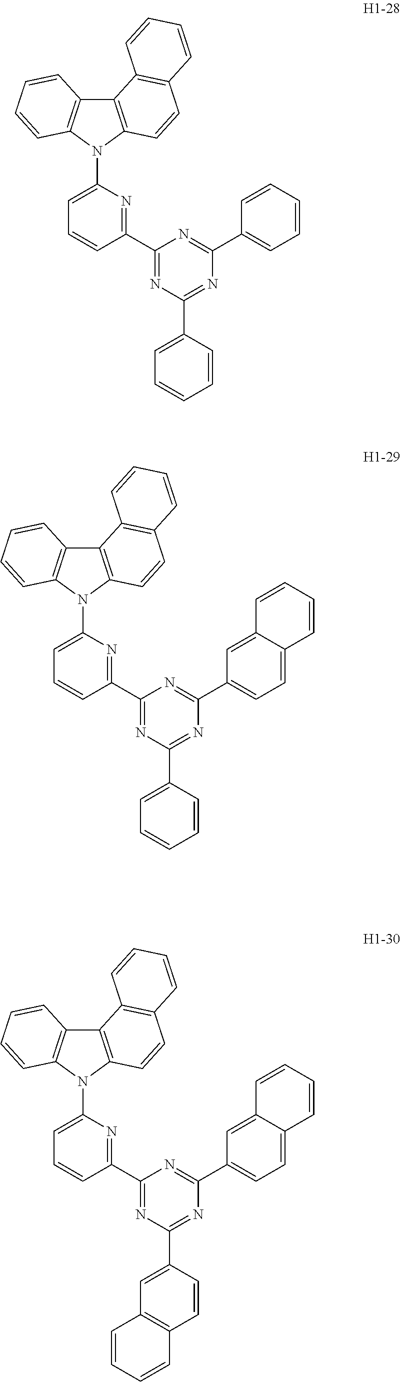 [SMILES: c1ccc(-c2nc(-c3ccccc3)nc(-c3cccc(-n4c5ccccc5c5c6ccccc6ccc54)n3)n2)cc1 ; c1ccc(-c2nc(-c3ccc4ccccc4c3)nc(-c3cccc(-n4c5ccccc5c5c6ccccc6ccc54)n3)n2)cc1 ; c1cc(-c2nc(-c3ccc4ccccc4c3)nc(-c3ccc4ccccc4c3)n2)nc(-n2c3ccccc3c3c4ccccc4ccc32)c1]

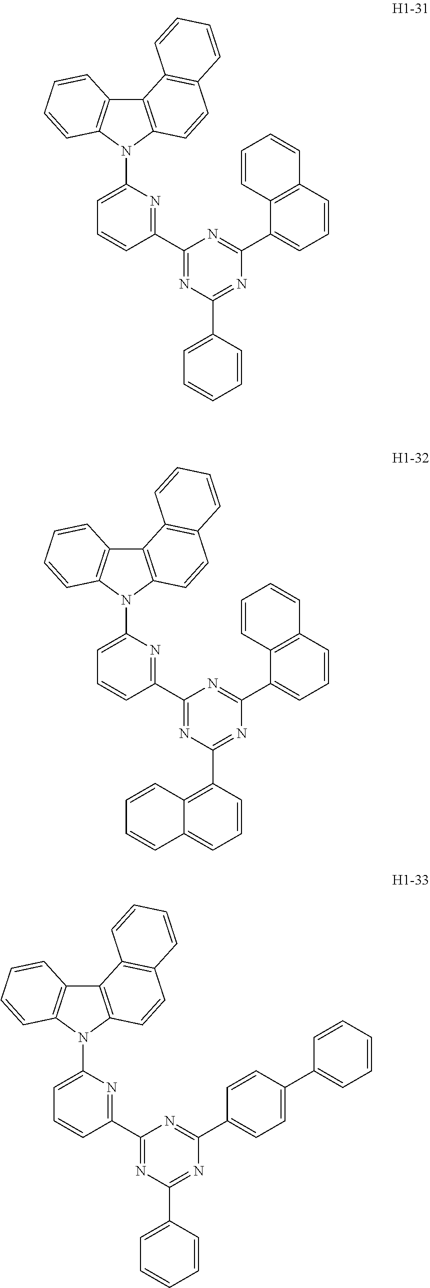 [SMILES: c1ccc(-c2nc(-c3cccc(-n4c5ccccc5c5c6ccccc6ccc54)n3)nc(-c3cccc4ccccc34)n2)cc1 ; c1cc(-c2nc(-c3cccc4ccccc34)nc(-c3cccc4ccccc34)n2)nc(-n2c3ccccc3c3c4ccccc4ccc32)c1 ; c1ccc(-c2ccc(-c3nc(-c4ccccc4)nc(-c4cccc(-n5c6ccccc6c6c7ccccc7ccc65)n4)n3)cc2)cc1]

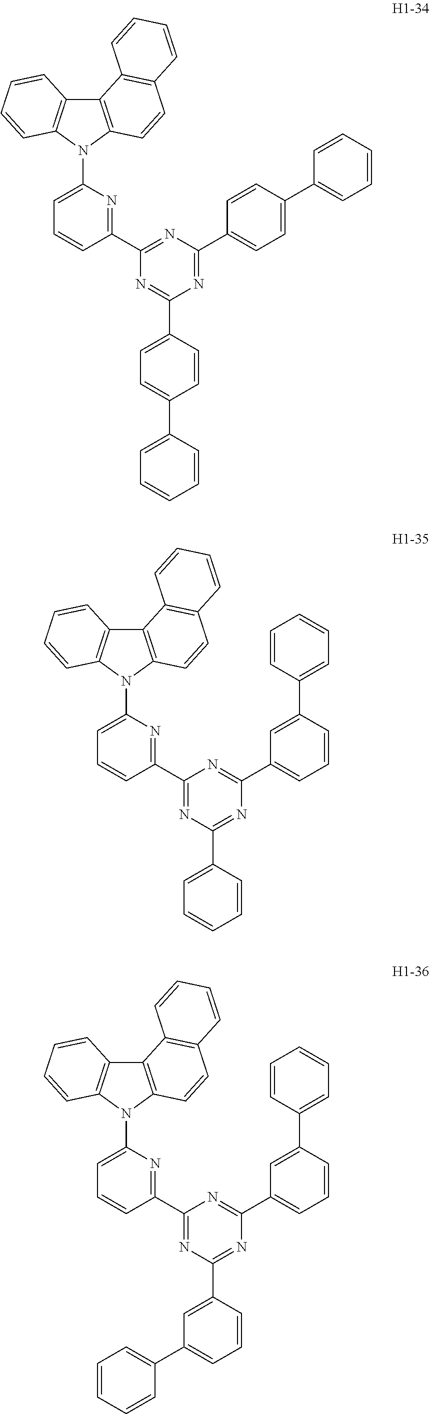 [SMILES: c1ccc(-c2ccc(-c3nc(-c4ccc(-c5ccccc5)cc4)nc(-c4cccc(-n5c6ccccc6c6c7ccccc7ccc65)n4)n3)cc2)cc1 ; c1ccc(-c2cccc(-c3nc(-c4ccccc4)nc(-c4cccc(-n5c6ccccc6c6c7ccccc7ccc65)n4)n3)c2)cc1 ; c1ccc(-c2cccc(-c3nc(-c4cccc(-c5ccccc5)c4)nc(-c4cccc(-n5c6ccccc6c6c7ccccc7ccc65)n4)n3)c2)cc1]

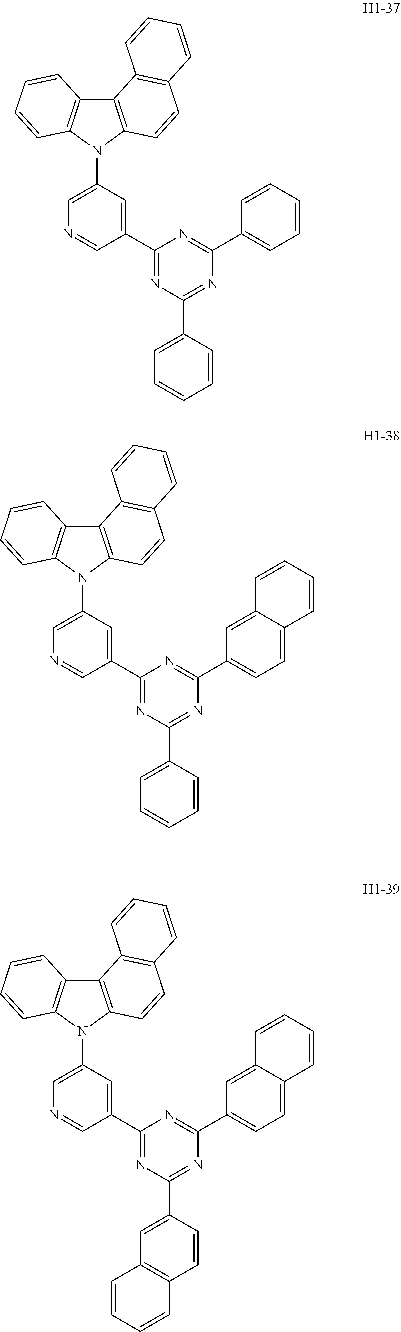 [SMILES: c1ccc(-c2nc(-c3ccccc3)nc(-c3cncc(-n4c5ccccc5c5c6ccccc6ccc54)c3)n2)cc1 ; c1ccc(-c2nc(-c3cncc(-n4c5ccccc5c5c6ccccc6ccc54)c3)nc(-c3ccc4ccccc4c3)n2)cc1 ; c1ccc2cc(-c3nc(-c4cncc(-n5c6ccccc6c6c7ccccc7ccc65)c4)nc(-c4ccc5ccccc5c4)n3)ccc2c1]

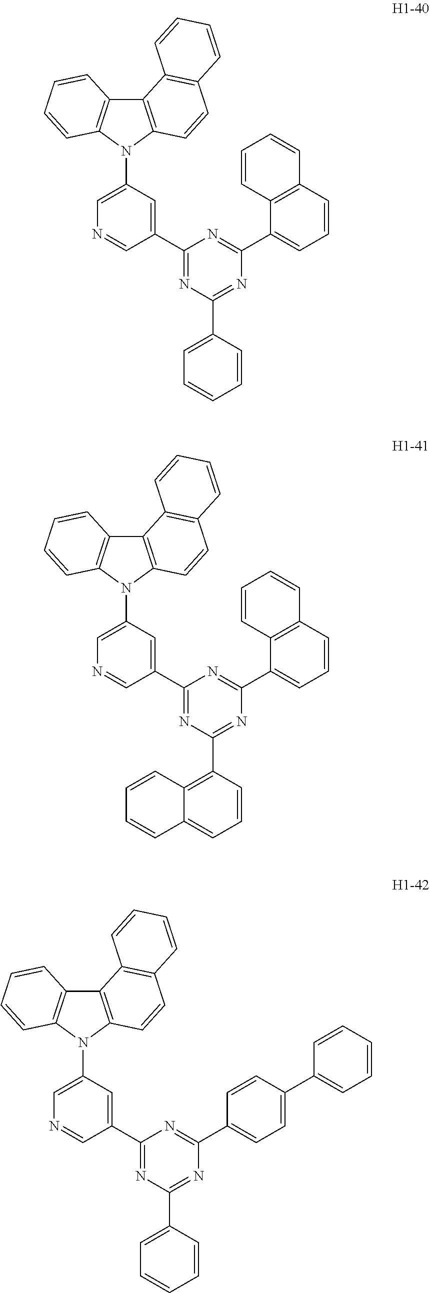 [SMILES: c1ccc(-c2nc(-c3cncc(-n4c5ccccc5c5c6ccccc6ccc54)c3)nc(-c3cccc4ccccc34)n2)cc1 ; c1ccc2c(-c3nc(-c4cncc(-n5c6ccccc6c6c7ccccc7ccc65)c4)nc(-c4cccc5ccccc45)n3)cccc2c1 ; c1ccc(-c2ccc(-c3nc(-c4ccccc4)nc(-c4cncc(-n5c6ccccc6c6c7ccccc7ccc65)c4)n3)cc2)cc1]

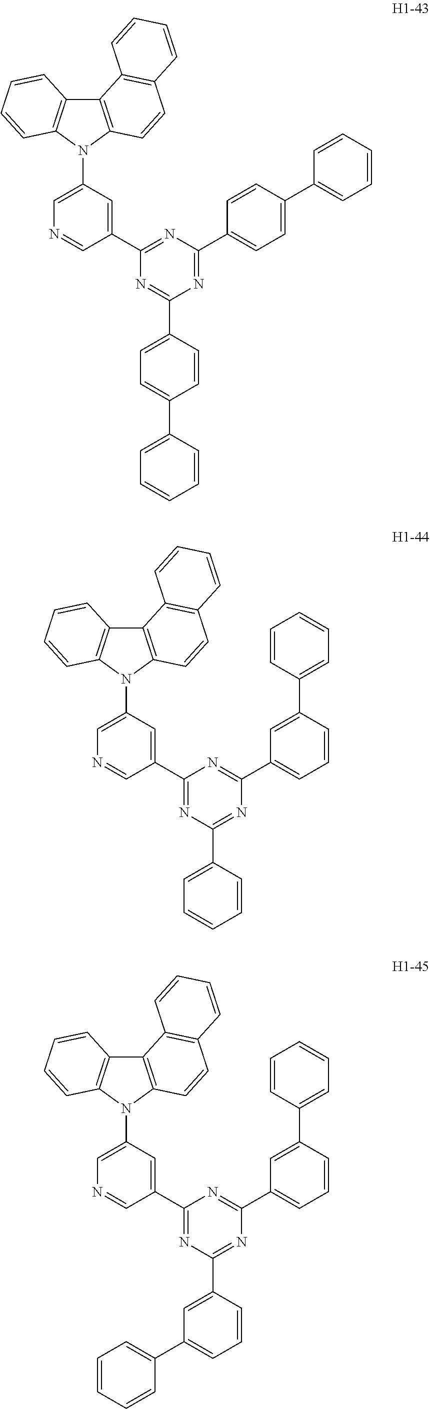

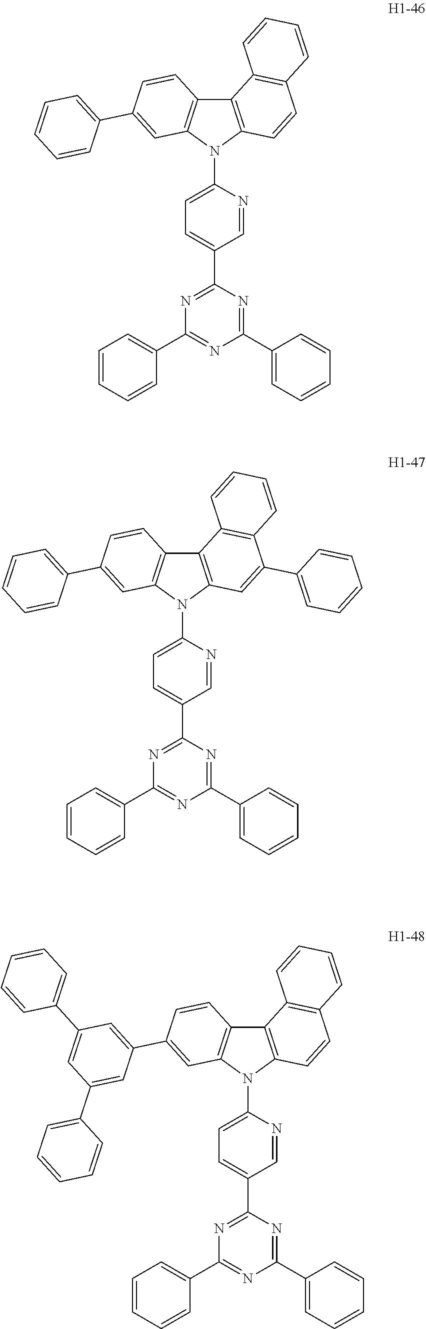 [SMILES: c1ccc(-c2ccc3c4c5ccccc5ccc4n(-c4ccc(-c5nc(-c6ccccc6)nc(-c6ccccc6)n5)cn4)c3c2)cc1 ; c1ccc(-c2ccc3c4c5ccccc5c(-c5ccccc5)cc4n(-c4ccc(-c5nc(-c6ccccc6)nc(-c6ccccc6)n5)cn4)c3c2)cc1 ; c1ccc(-c2cc(-c3ccccc3)cc(-c3ccc4c5c6ccccc6ccc5n(-c5ccc(-c6nc(-c7ccccc7)nc(-c7ccccc7)n6)cn5)c4c3)c2)cc1]

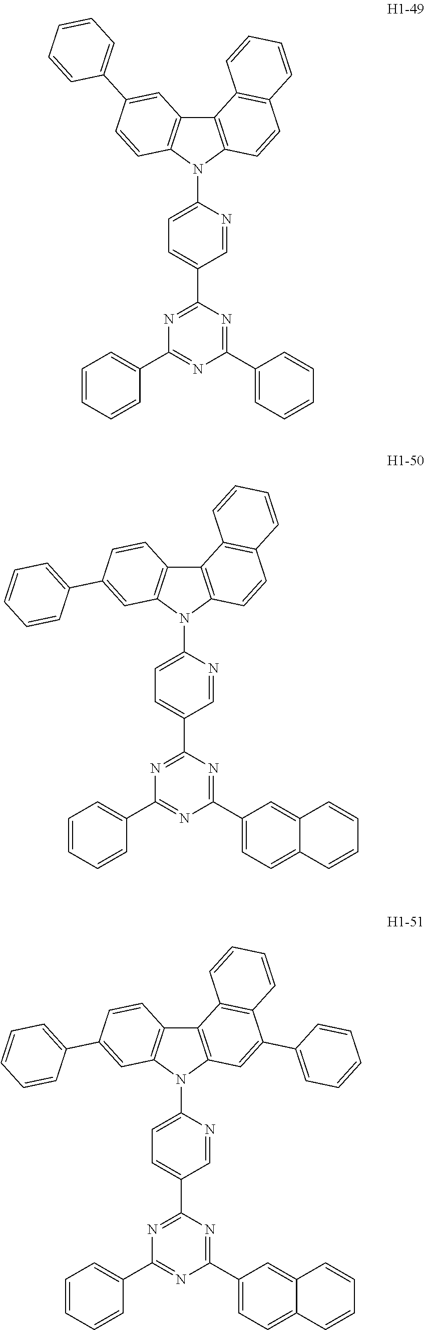 [SMILES: c1ccc(-c2ccc3c(c2)c2c4ccccc4ccc2n3-c2ccc(-c3nc(-c4ccccc4)nc(-c4ccccc4)n3)cn2)cc1 ; c1ccc(-c2ccc3c4c5ccccc5ccc4n(-c4ccc(-c5nc(-c6ccccc6)nc(-c6ccc7ccccc7c6)n5)cn4)c3c2)cc1 ; c1ccc(-c2ccc3c4c5ccccc5c(-c5ccccc5)cc4n(-c4ccc(-c5nc(-c6ccccc6)nc(-c6ccc7ccccc7c6)n5)cn4)c3c2)cc1]

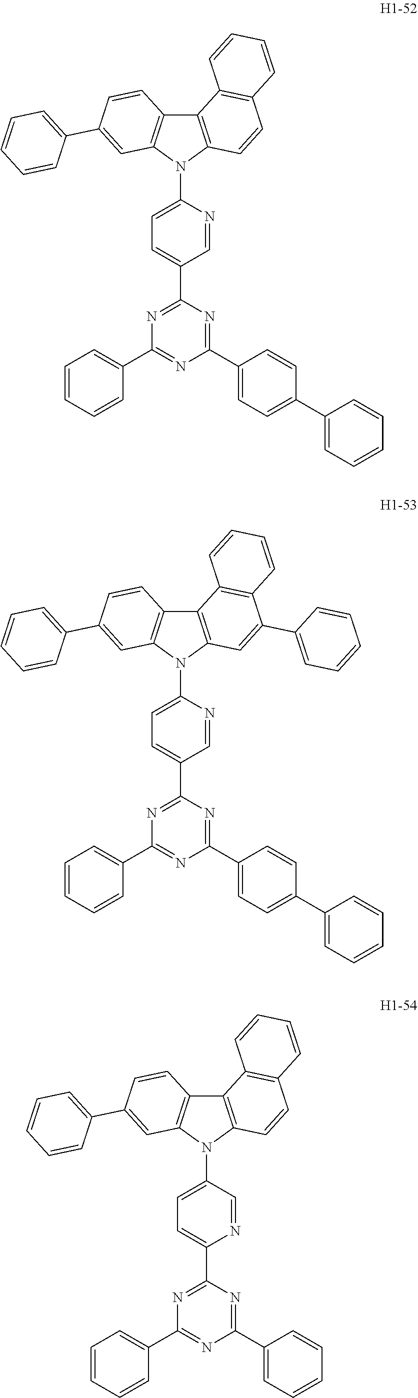 [SMILES: c1ccc(-c2ccc(-c3nc(-c4ccccc4)nc(-c4ccc(-n5c6cc(-c7ccccc7)ccc6c6c7ccccc7ccc65)nc4)n3)cc2)cc1 ; c1ccc(-c2ccc(-c3nc(-c4ccccc4)nc(-c4ccc(-n5c6cc(-c7ccccc7)ccc6c6c7ccccc7c(-c7ccccc7)cc65)nc4)n3)cc2)cc1 ; c1ccc(-c2ccc3c4c5ccccc5ccc4n(-c4ccc(-c5nc(-c6ccccc6)nc(-c6ccccc6)n5)nc4)c3c2)cc1]

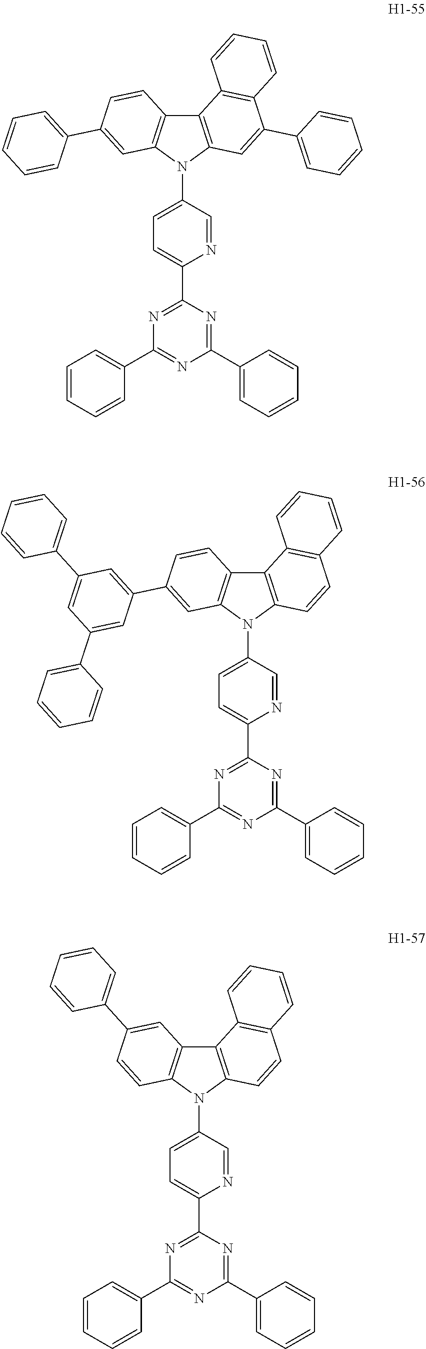 [SMILES: c1ccc(-c2ccc3c4c5ccccc5c(-c5ccccc5)cc4n(-c4ccc(-c5nc(-c6ccccc6)nc(-c6ccccc6)n5)nc4)c3c2)cc1 ; c1ccc(-c2cc(-c3ccccc3)cc(-c3ccc4c5c6ccccc6ccc5n(-c5ccc(-c6nc(-c7ccccc7)nc(-c7ccccc7)n6)nc5)c4c3)c2)cc1 ; c1ccc(-c2ccc3c(c2)c2c4ccccc4ccc2n3-c2ccc(-c3nc(-c4ccccc4)nc(-c4ccccc4)n3)nc2)cc1]

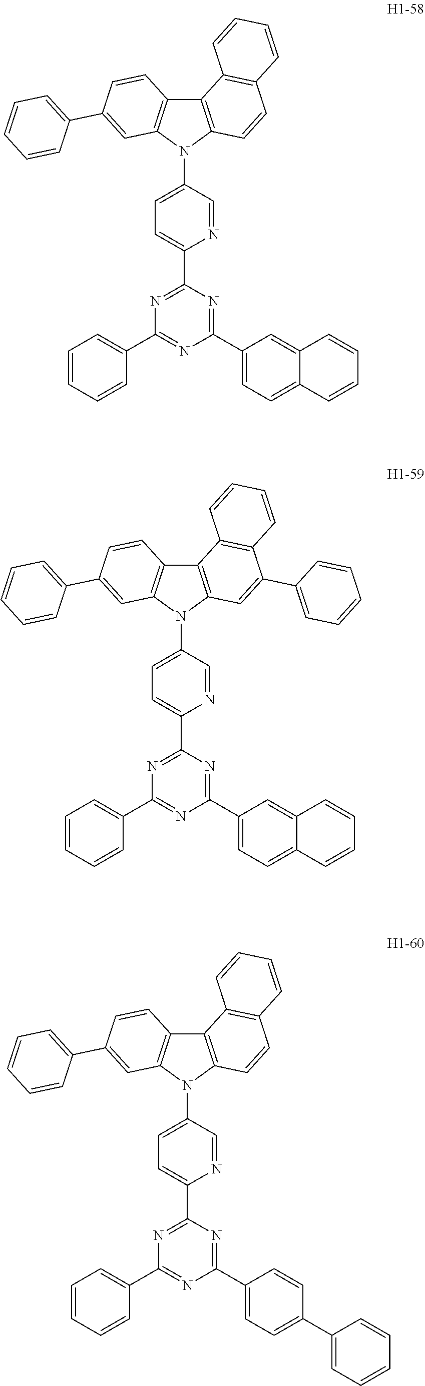 [SMILES: c1ccc(-c2ccc3c4c5ccccc5ccc4n(-c4ccc(-c5nc(-c6ccccc6)nc(-c6ccc7ccccc7c6)n5)nc4)c3c2)cc1 ; c1ccc(-c2ccc3c4c5ccccc5c(-c5ccccc5)cc4n(-c4ccc(-c5nc(-c6ccccc6)nc(-c6ccc7ccccc7c6)n5)nc4)c3c2)cc1 ; c1ccc(-c2ccc(-c3nc(-c4ccccc4)nc(-c4ccc(-n5c6cc(-c7ccccc7)ccc6c6c7ccccc7ccc65)cn4)n3)cc2)cc1]

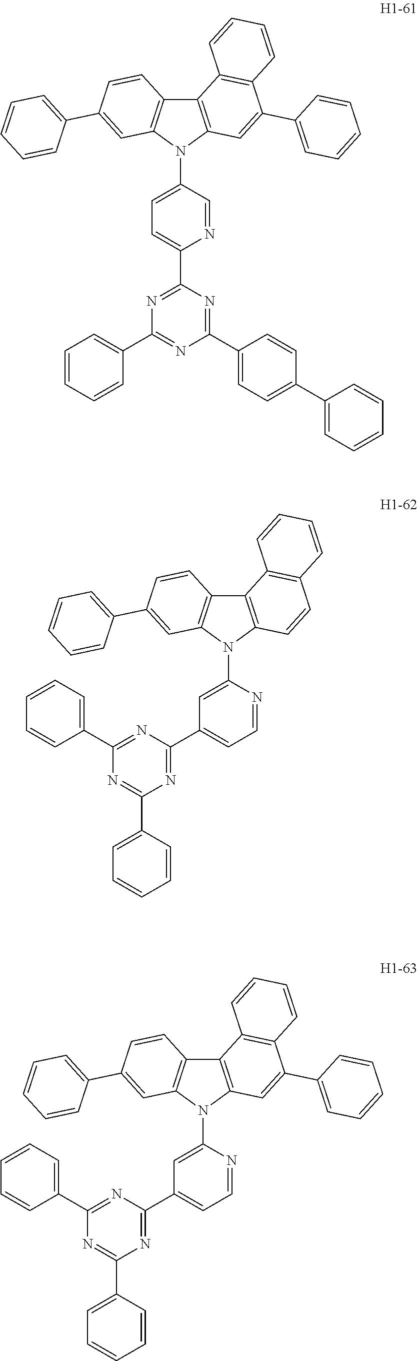 [SMILES: c1ccc(-c2ccc(-c3nc(-c4ccccc4)nc(-c4ccc(-n5c6cc(-c7ccccc7)ccc6c6c7ccccc7c(-c7ccccc7)cc65)cn4)n3)cc2)cc1 ; c1ccc(-c2ccc3c4c5ccccc5ccc4n(-c4cc(-c5nc(-c6ccccc6)nc(-c6ccccc6)n5)ccn4)c3c2)cc1 ; c1ccc(-c2ccc3c4c5ccccc5c(-c5ccccc5)cc4n(-c4cc(-c5nc(-c6ccccc6)nc(-c6ccccc6)n5)ccn4)c3c2)cc1]

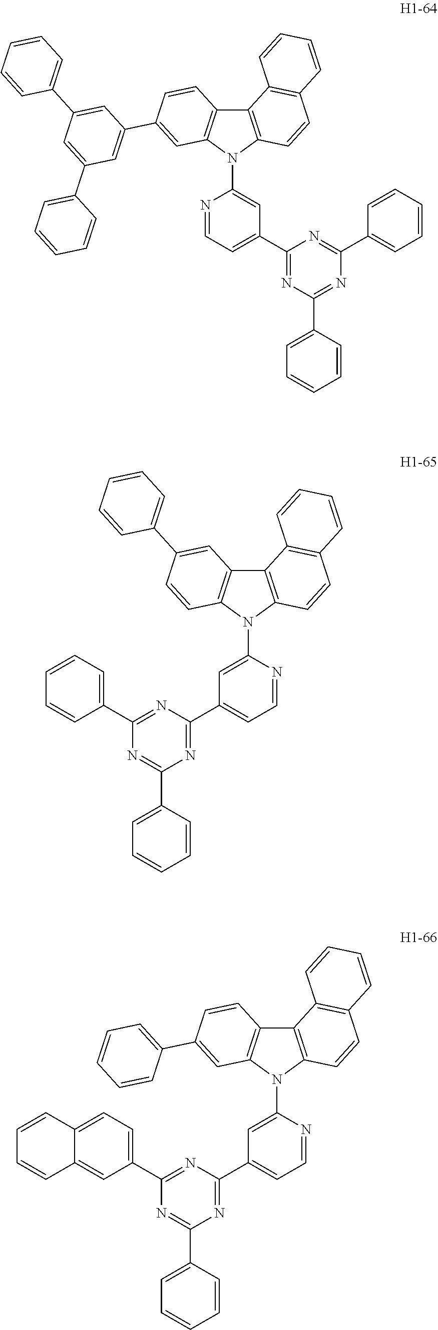 [SMILES: c1ccc(-c2cc(-c3ccccc3)cc(-c3ccc4c5c6ccccc6ccc5n(-c5cc(-c6nc(-c7ccccc7)nc(-c7ccccc7)n6)ccn5)c4c3)c2)cc1 ; c1ccc(-c2ccc3c(c2)c2c4ccccc4ccc2n3-c2cc(-c3nc(-c4ccccc4)nc(-c4ccccc4)n3)ccn2)cc1 ; c1ccc(-c2ccc3c4c5ccccc5ccc4n(-c4cc(-c5nc(-c6ccccc6)nc(-c6ccc7ccccc7c6)n5)ccn4)c3c2)cc1]

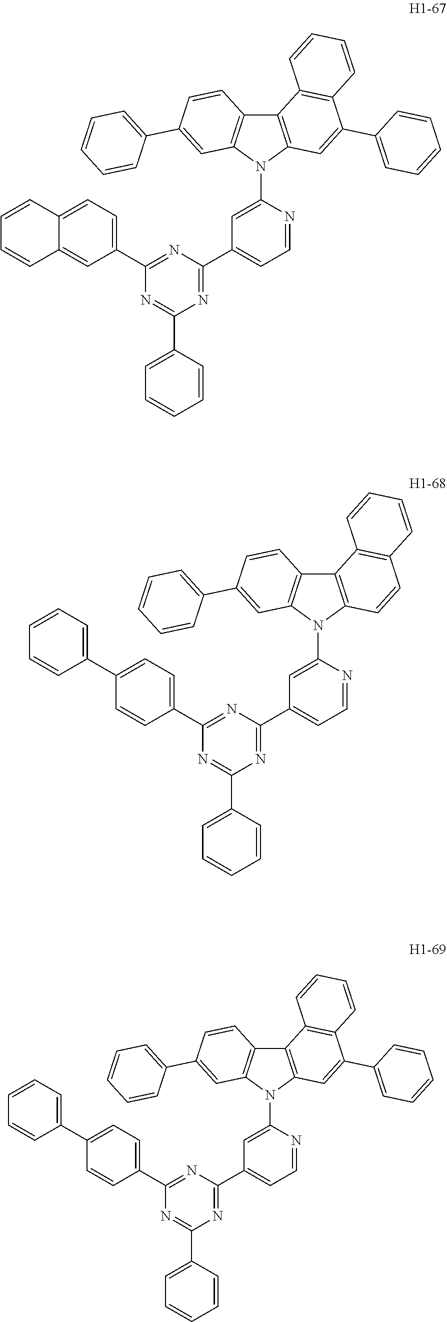 [SMILES: c1ccc(-c2ccc3c4c5ccccc5c(-c5ccccc5)cc4n(-c4cc(-c5nc(-c6ccccc6)nc(-c6ccc7ccccc7c6)n5)ccn4)c3c2)cc1 ; c1ccc(-c2ccc(-c3nc(-c4ccccc4)nc(-c4ccnc(-n5c6cc(-c7ccccc7)ccc6c6c7ccccc7ccc65)c4)n3)cc2)cc1 ; c1ccc(-c2ccc(-c3nc(-c4ccccc4)nc(-c4ccnc(-n5c6cc(-c7ccccc7)ccc6c6c7ccccc7c(-c7ccccc7)cc65)c4)n3)cc2)cc1]

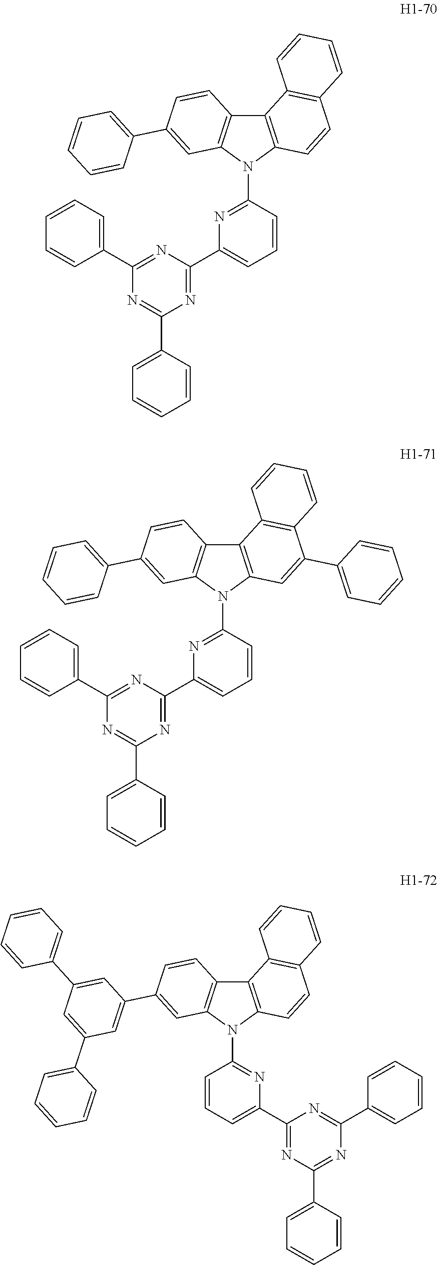 [SMILES: c1ccc(-c2ccc3c4c5ccccc5ccc4n(-c4cccc(-c5nc(-c6ccccc6)nc(-c6ccccc6)n5)n4)c3c2)cc1 ; c1ccc(-c2ccc3c4c5ccccc5c(-c5ccccc5)cc4n(-c4cccc(-c5nc(-c6ccccc6)nc(-c6ccccc6)n5)n4)c3c2)cc1 ; c1ccc(-c2cc(-c3ccccc3)cc(-c3ccc4c5c6ccccc6ccc5n(-c5cccc(-c6nc(-c7ccccc7)nc(-c7ccccc7)n6)n5)c4c3)c2)cc1]

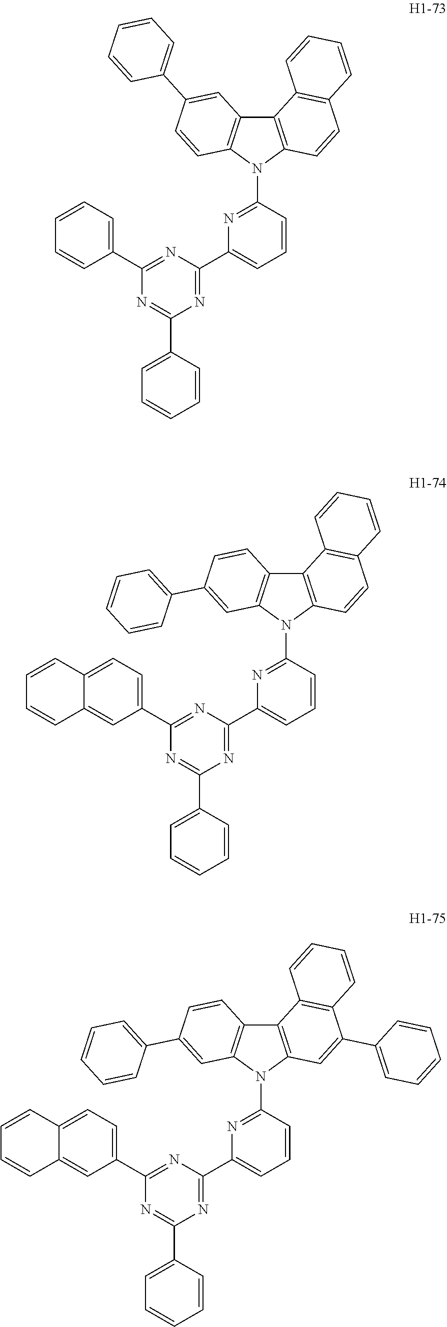 [SMILES: c1ccc(-c2ccc3c(c2)c2c4ccccc4ccc2n3-c2cccc(-c3nc(-c4ccccc4)nc(-c4ccccc4)n3)n2)cc1 ; c1ccc(-c2ccc3c4c5ccccc5ccc4n(-c4cccc(-c5nc(-c6ccccc6)nc(-c6ccc7ccccc7c6)n5)n4)c3c2)cc1 ; c1ccc(-c2ccc3c4c5ccccc5c(-c5ccccc5)cc4n(-c4cccc(-c5nc(-c6ccccc6)nc(-c6ccc7ccccc7c6)n5)n4)c3c2)cc1]

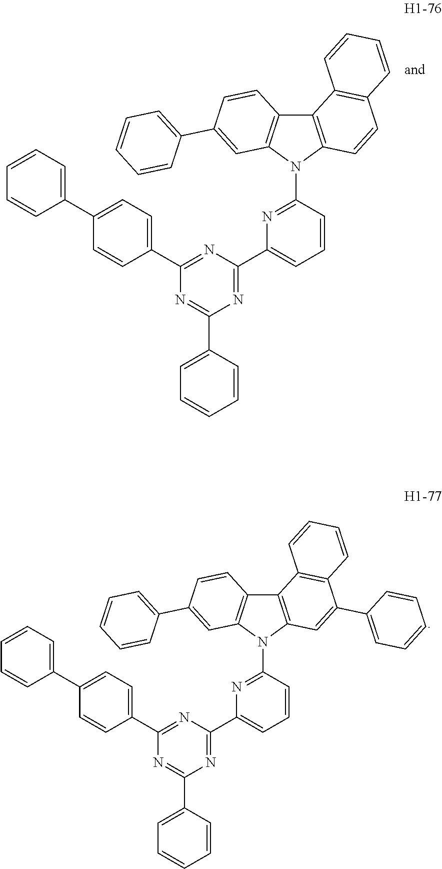 [SMILES: c1ccc(-c2ccc(-c3nc(-c4ccccc4)nc(-c4cccc(-n5c6cc(-c7ccccc7)ccc6c6c7ccccc7ccc65)n4)n3)cc2)cc1 ; c1ccc(-c2ccc(-c3nc(-c4ccccc4)nc(-c4cccc(-n5c6cc(-c7ccccc7)ccc6c6c7ccccc7c(-c7ccccc7)cc65)n4)n3)cc2)cc1]

7. The organic electroluminescent device according to claim 1, wherein the second host compound of formula 7 is represented by any one of the following formulae 8 to 11: 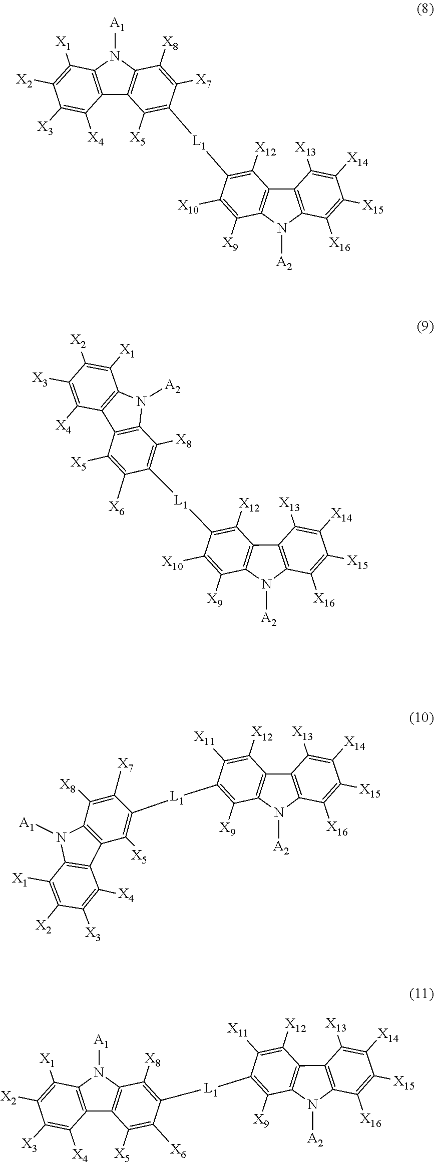
wherein A₁, A₂, L₁ and X₁ to X₁₆ are as defined in claim 8.

8. The organic electroluminescent device according to claim 1, wherein A₁ is selected from the group consisting of a substituted or unsubstituted phenyl, a substituted or unsubstituted biphenyl, a substituted or unsubstituted terphenyl, a substituted or unsubstituted naphthyl, a substituted or unsubstituted binaphthyl, a substituted or unsubstituted phenylnaphthyl, a substituted or unsubstituted naphthylphenyl, a substituted or unsubstituted fluorenyl, a substituted or unsubstituted phenylfluorenyl, a substituted or unsubstituted benzofluorenyl, a substituted or unsubstituted dibenzofluorenyl, a substituted or unsubstituted phenanthrenyl, a substituted or unsubstituted phenylphenanthrenyl, a substituted or unsubstituted anthracenyl, a substituted or unsubstituted indenyl, a substituted or unsubstituted triphenylenyl, a substituted or unsubstituted pyrenyl, a substituted or unsubstituted tetracenyl, a substituted or unsubstituted perylenyl, a substituted or unsubstituted chrysenyl, a substituted or unsubstituted naphthacenyl, a substituted or unsubstituted fluoranthenyl and a substituted or unsubstituted spirobifluorenyl.

9. The organic electroluminescent device according to claim 1, wherein L₁ represents a single bond, a substituted or unsubstituted phenylene, a substituted or unsubstituted naphthylene, or a substituted or unsubstituted biphenylene.

10. The organic electroluminescent device according to claim 1, wherein the second host compound represented by formula 7 is selected from the group consisting of: 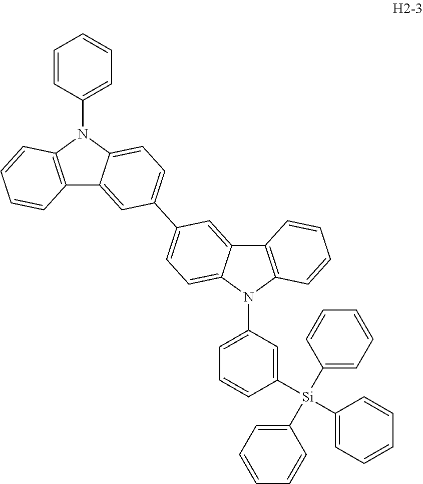 [SMILES: c1ccc(-n2c3ccccc3c3cc(-c4ccc5c(c4)c4ccccc4n5-c4cccc([Si](c5ccccc5)(c5ccccc5)c5ccccc5)c4)ccc32)cc1] 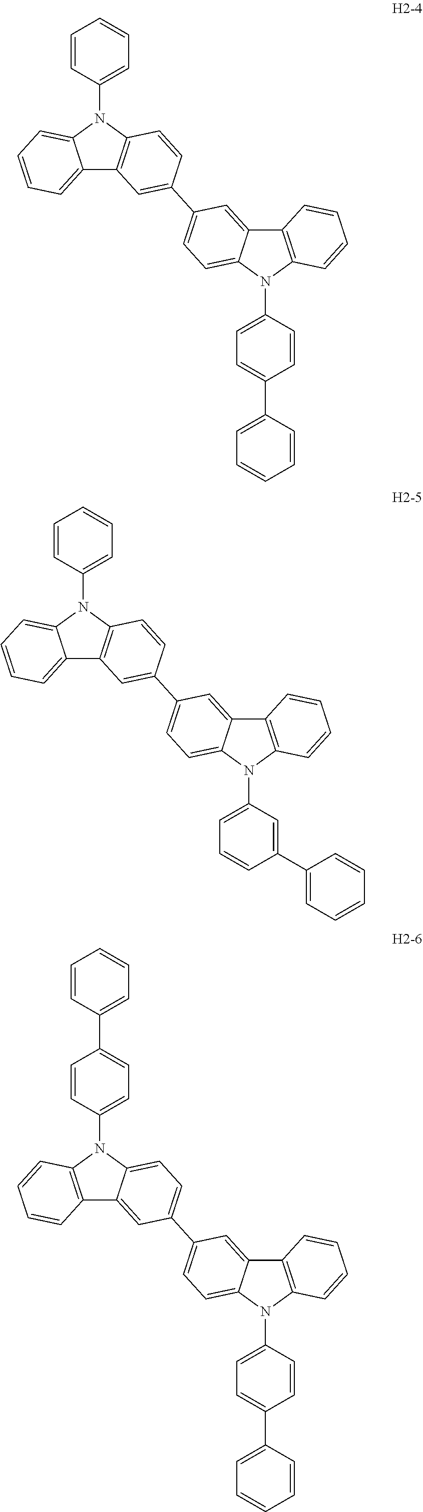 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1] 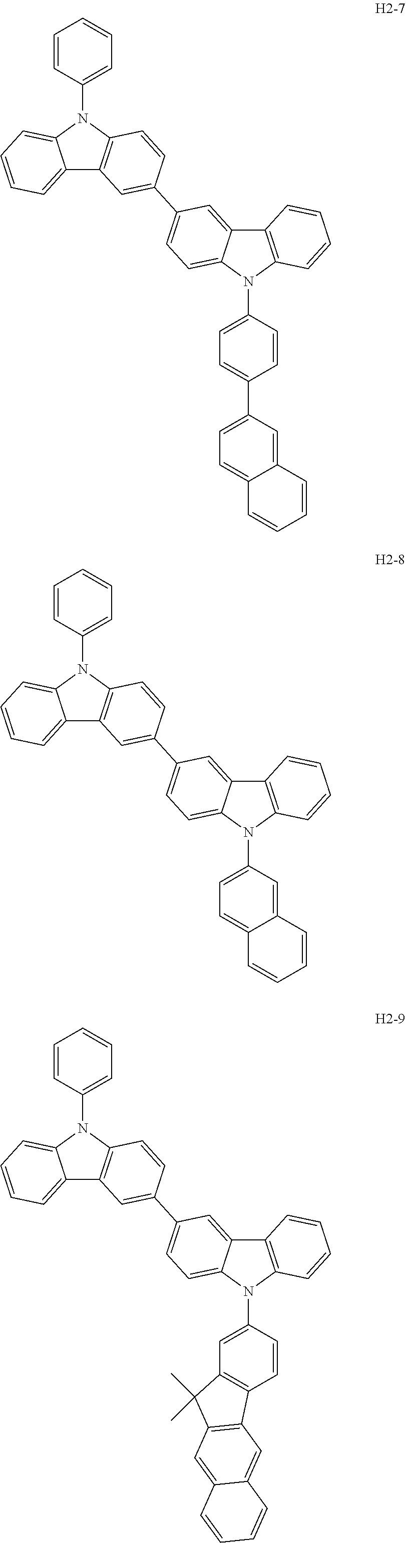 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21] 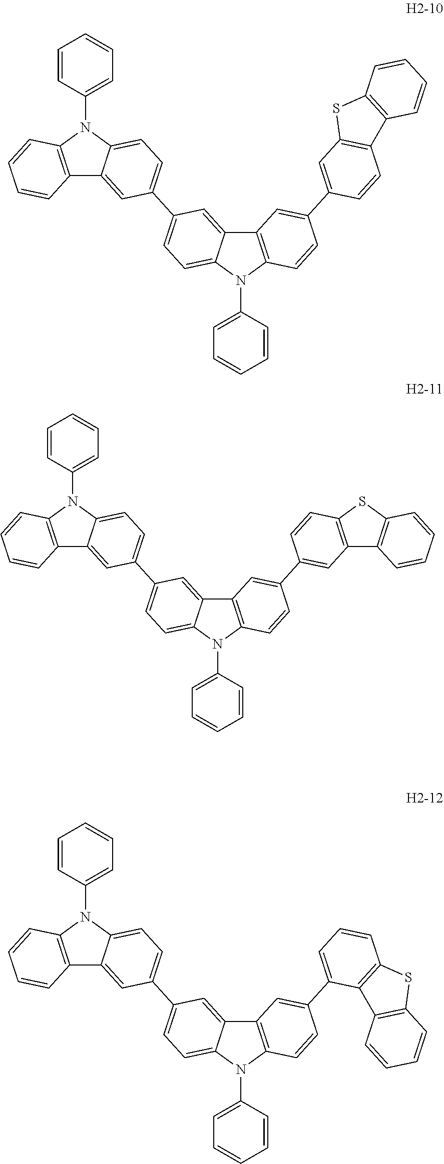 [SMILES: c1ccc(-n2c3ccccc3c3cc(-c4ccc5c(c4)c4cc(-c6ccc7c(c6)sc6ccccc67)ccc4n5-c4ccccc4)ccc32)cc1 ; c1ccc(-n2c3ccccc3c3cc(-c4ccc5c(c4)c4cc(-c6ccc7sc8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1] 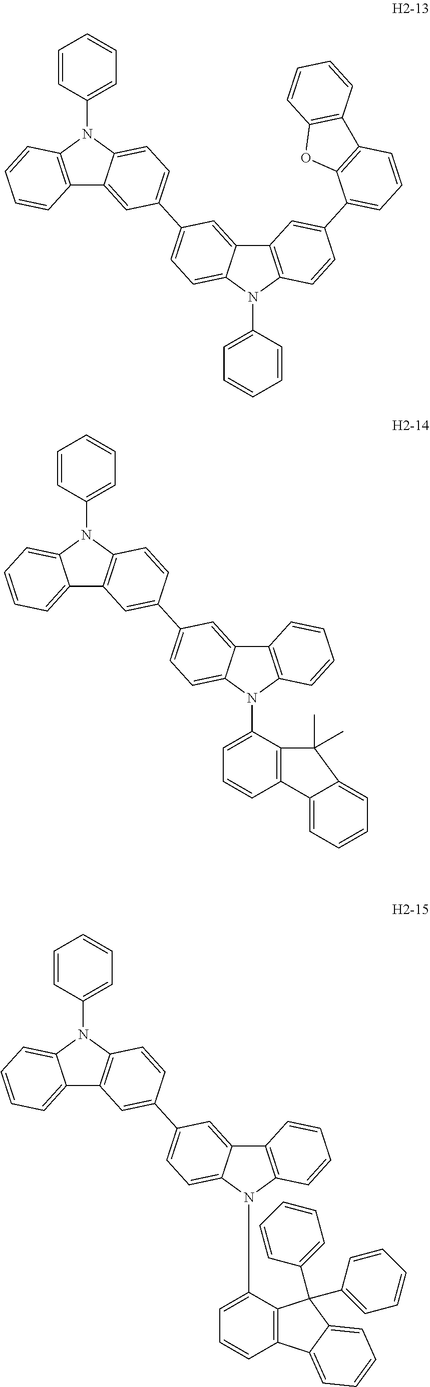 [SMILES: c1ccc(-n2c3ccccc3c3cc(-c4ccc5c(c4)c4cc(-c6cccc7c6oc6ccccc67)ccc4n5-c4ccccc4)ccc32)cc1 ; CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1] 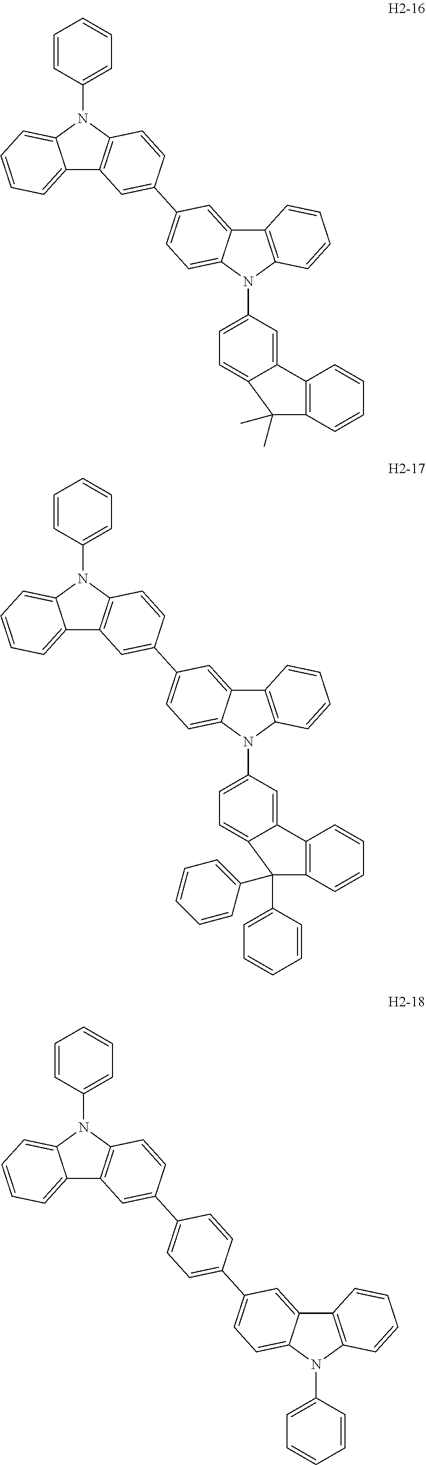 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1 ; c1ccc(-n2c3ccccc3c3cc(-c4ccc(-c5ccc6c(c5)c5ccccc5n6-c5ccccc5)cc4)ccc32)cc1] 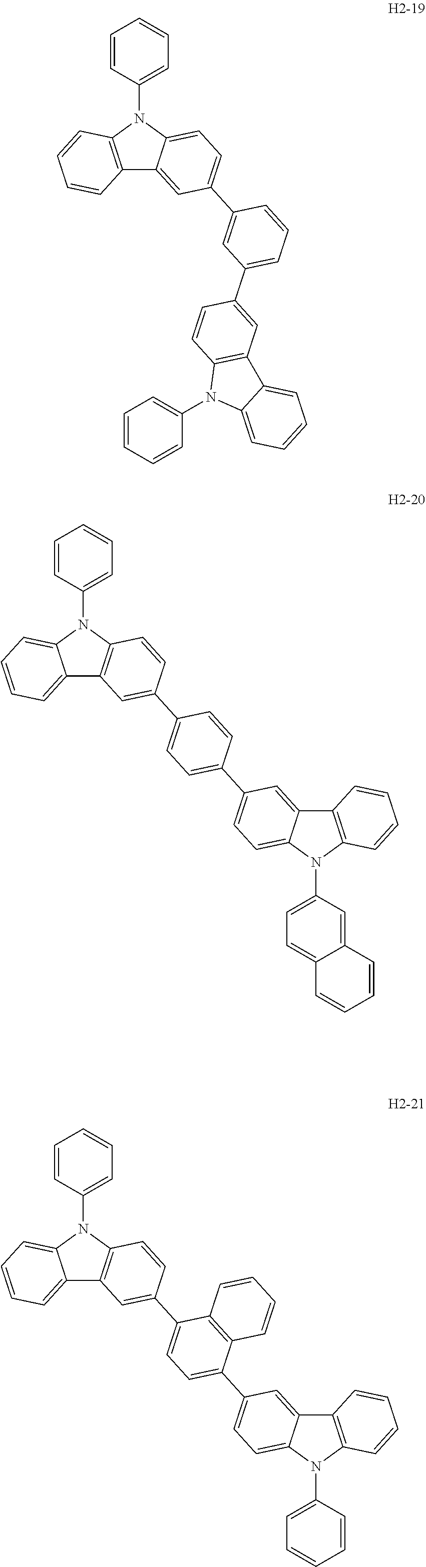 [SMILES: c1ccc(-n2c3ccccc3c3cc(-c4cccc(-c5ccc6c(c5)c5ccccc5n6-c5ccccc5)c4)ccc32)cc1 ; c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4ccc(-c5ccc6c(c5)c5ccccc5n6-c5ccccc5)c5ccccc45)ccc32)cc1] 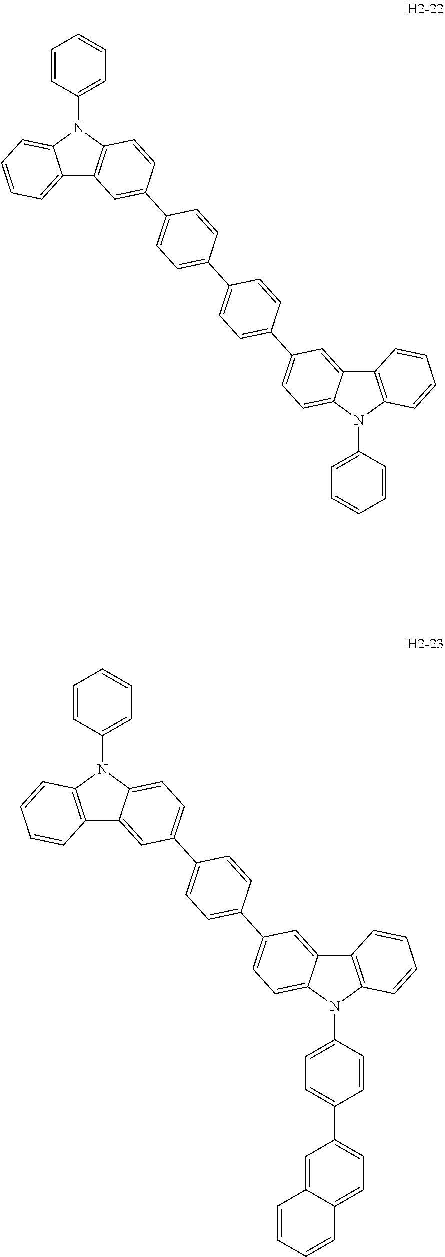 [SMILES: c1ccc(-n2c3ccccc3c3cc(-c4ccc(-c5ccc(-c6ccc7c(c6)c6ccccc6n7-c6ccccc6)cc5)cc4)ccc32)cc1 ; c1ccc(-n2c3ccccc3c3cc(-c4ccc(-c5ccc6c(c5)c5ccccc5n6-c5ccc(-c6ccc7ccccc7c6)cc5)cc4)ccc32)cc1] 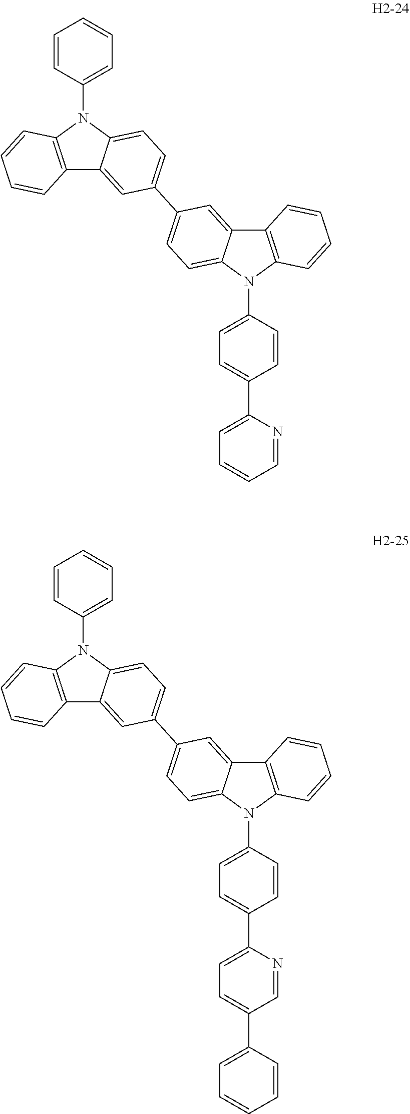 [SMILES: c1ccc(-n2c3ccccc3c3cc(-c4ccc5c(c4)c4ccccc4n5-c4ccc(-c5ccccn5)cc4)ccc32)cc1 ; c1ccc(-c2ccc(-c3ccc(-n4c5ccccc5c5cc(-c6ccc7c(c6)c6ccccc6n7-c6ccccc6)ccc54)cc3)nc2)cc1] 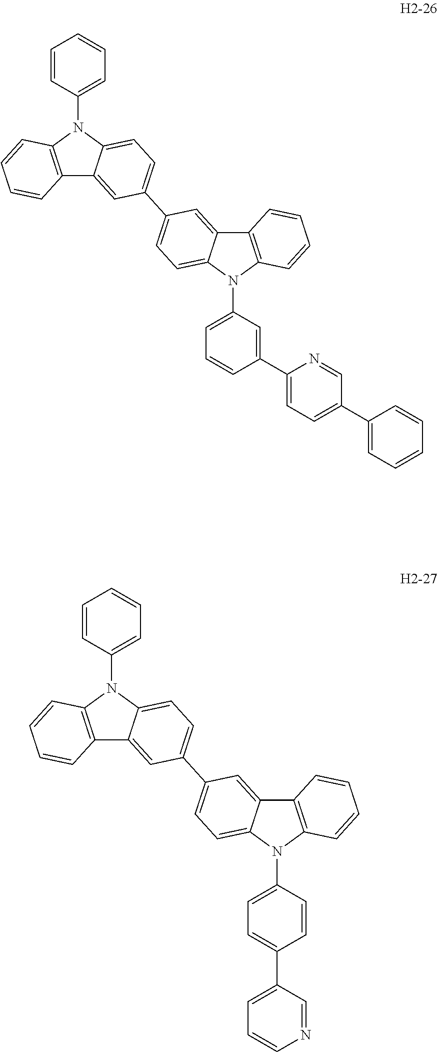 [SMILES: c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1] 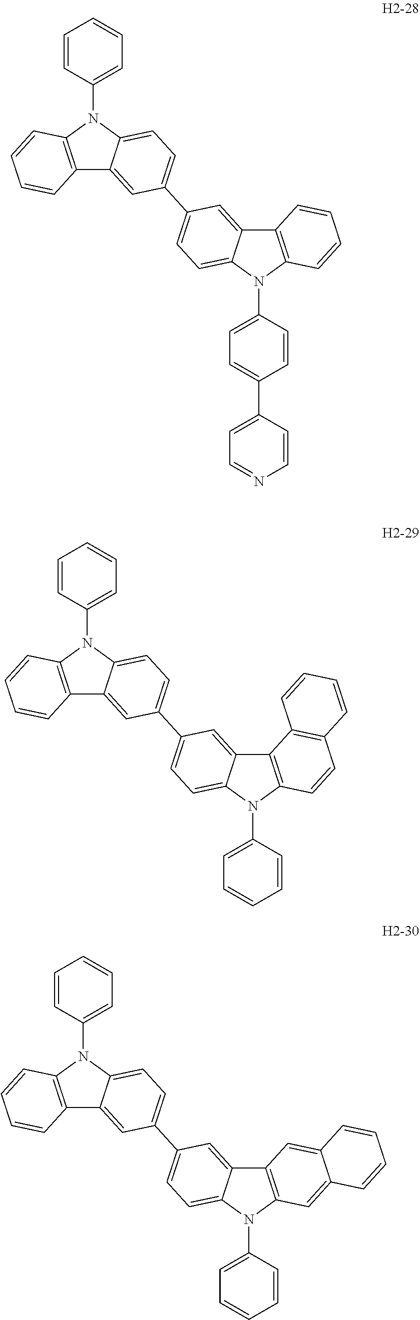 [SMILES: c1ccc(-n2c3ccccc3c3cc(-c4ccc5c(c4)c4ccccc4n5-c4ccc(-c5ccncc5)cc4)ccc32)cc1 ; c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1] 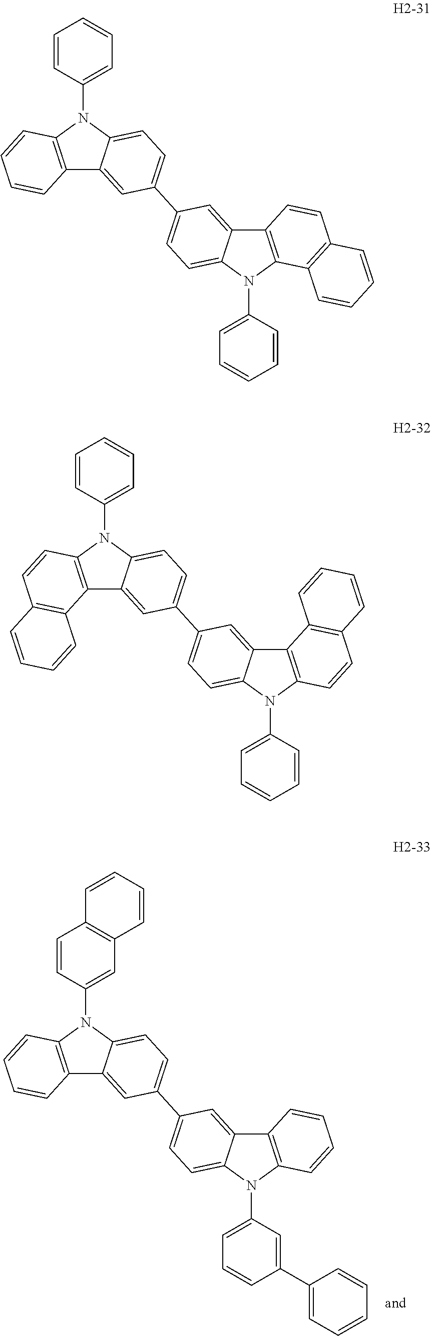 [SMILES: c1ccc(-n2c3ccccc3c3cc(-c4ccc5c(c4)c4ccc6ccccc6c4n5-c4ccccc4)ccc32)cc1 ; c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1] 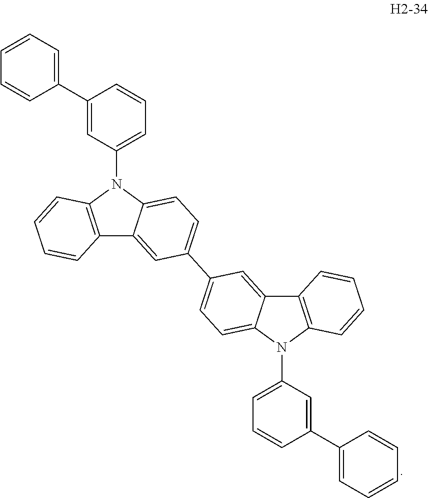 [SMILES: c1ccc(-c2cccc(-n3c4ccccc4c4cc(-c5ccc6c(c5)c5ccccc5n6-c5cccc(-c6ccccc6)c5)ccc43)c2)cc1]
